(19) United States
(12) Patent Application Publication
(10) Pub. No.: US 2010/0105125 A1
(43) Pub. Date: Apr. 29, 2010

(54) SYSTEMS, APPARATUSES AND METHODS FOR CULTIVATING MICROORGANISMS AND MITIGATION OF GASES

(75) Inventor: John W. Haley, III, Providence, RI (US)

Correspondence Address: WILDMAN HARROLD ALLEN & DIXON LLP, 225 WEST WACKER DRIVE, SUITE 2800, CHICAGO, IL, 60606, US

(73) Assignee: BioProcessH20 LLC, Portsmouth, RI (US)

(21) Appl. No.: 12/605,121

(22) Filed: Oct. 23, 2009

Related U.S. Application Data
(60) Provisional application No. 61/108,183, filed on Oct. 24, 2008.
(60) Provisional application No. 61/175,950, filed on May 6, 2009.
(60) Provisional application No. 61/241,520, filed on Sep. 11, 2009.

(51) Int. Cl. C12N 1/12; C12M 1/24

(52) U.S. Cl. 435/257.1; 435/304.1

(57) ABSTRACT

Systems, apparatuses, and methods are provided for cultivating microorganisms. In one example, a system may include a plurality of containers for cultivating microorganisms therein. Each container may be adapted to contain water and may include media disposed therein and at least partially submerged in the water. The media may be adapted to support microorganisms during cultivation and a concentration of microorganisms supported by the media may be higher than a concentration of microorganisms suspended in the water.

### RELATED APPLICATIONS

The present application claims the benefit of co-pending U.S. Provisional Patent Application Nos. 61/108,183, filed Oct. 24, 2008, 61/175,950, filed May 6, 2009, and 61/241,520, filed Sep. 11, 2009, the contents of all are hereby incorporated herein by reference.

### FIELD OF THE INVENTION

The present invention generally relates to systems, apparatuses, and methods for cultivating microorganisms and mitigating gases and, more particularly, to systems, apparatuses, and methods for cultivating algae for use in producing lipids and other cellular products that may be used directly or in a refined state to produce other products such as biodiesel fuel or other fuels, and for mitigation of gases, such as carbon dioxide.

### BACKGROUND

Microorganisms such as algae have previously been grown for the production of fuels, such as biodiesel fuel. However, microorganism growth has been counterproductive due to the high costs and energy demands required to produce the microorganisms. In most cases, the costs and energy demands exceed the revenue and energy derived from the microorganism growth processes. Additionally, microorganism growth processes are inefficient at cultivating high levels of microorganisms in a relatively short period of time. Accordingly, a need exists for systems, apparatuses, and methods for growing microorganisms, such as algae, that have low production costs and energy demands, and produce large quantities of microorganisms in an efficient manner, thereby facilitating high levels of fuel production.

### SUMMARY

In one example, a system for cultivating microorganisms is provided.

In another example, a container for cultivating microorganisms is provided.

In yet another example, a method for cultivating microorganisms is provided.

In still another example, a system, a container, or a method is provided for cultivating algae for use in fuel production.

In a further example, a container for cultivating a microorganism is provided and includes a housing for containing water and the microorganism, an inlet defined in the housing for permitting gas to enter the housing, and a media at least partially positioned within the housing and including an elongated member and a plurality of loop members extending from the elongated member.

In yet a further example, a container for cultivating a microorganism is provided and includes a housing for containing water and the microorganism, an inlet defined in the housing for permitting gas to enter the housing, a frame at least partially positioned within the housing and including a first portion and a second portion, the first portion is spaced apart from the second portion, and a media at least partially positioned within the housing and supported by and extending between the first and second portions.

In still a further example, a container for cultivating a microorganism is provided and includes a housing for containing water and a microorganism, and a media positioned within the housing and in contact with an interior surface of the housing, the media is movable between a first position and a second position within the housing, and the media maintains contact with the interior surface of the housing as the media moves between the first and second positions.

In another example, a method for cultivating a microorganism is provided and includes providing a container for containing water and the microorganism, positioning a media at least partially within the container and in contact with an interior surface of the container, moving the media within the container from a first position to a second position, and maintaining the media in contact with the interior surface of the housing as the media moves from the first position to the second position.

In yet another example, a container for cultivating a microorganism is provided and includes a housing for containing water and the microorganism, a frame at least partially positioned within the housing and including a first portion and a second portion, the first portion is spaced apart from the second portion, and the frame is rotatable relative to the housing, a first media segment coupled to and extending between the first and second portions of the frame, and a second media segment coupled to and extending between the first and second portions of the frame, at least a portion of the first media segment and at least a portion of the second media segment are spaced apart from each other.

In still another example, a container for cultivating a microorganism is provided and includes a housing for containing water and the microorganism, the housing including a sidewall. The container also including a plurality of media segments at least partially positioned within the housing and including a first pair of media segments spaced apart from each other a first distance and a second pair of media segments spaced apart from each other a second distance, the first distance is greater than the second distance, and the first pair of media segments is positioned closer to the sidewall than the second pair of media segments.

In a further example, a container for cultivating a microorganism is provided and includes a housing for containing water and the microorganism, a frame at least partially positioned within the housing and including two spaced apart frame portions, and a media at least partially positioned within the housing and extending between the two spaced apart frame portions, the frame is constructed of a first material more rigid than a second material of which the media is constructed.

In yet a further example, a container for cultivating a microorganism is provided and includes a housing for containing water and the microorganism, a frame at least partially positioned within the housing and movable relative to the housing, a drive member coupled to the frame and adapted to move the frame at a first speed and a second speed, the first speed is different than the second speed, and a media at least partially positioned within the housing and coupled to the frame.

In still a further example, a container for cultivating a microorganism is provided and includes a housing for containing water and the microorganism, a frame at least partially positioned within the housing and movable relative to the housing, the frame including two spaced apart frame portions, a drive member coupled to the frame for moving the frame, and a media at least partially positioned within the housing and extending between the two spaced apart frame portions.

In another example, a container for cultivating a microorganism is provided and includes a housing for containing water and the microorganism, a frame at least partially positioned within the housing and movable relative to the housing, a media coupled to the frame, and an artificial lighting element for emitting light into an interior of the housing.

In yet another example, a container for cultivating a microorganism is provided and includes a housing for containing water and the microorganism, an artificial light source for emitting light into an interior of the housing, a member associated with the artificial light source and through which the light emitted from the artificial light source passes, and a wiping element at least partially positioned within the housing and in contact with the member, the wiping element is movable relative to the member to wipe against the member.

In still another example, a container for cultivating a microorganism is provided and includes a housing for containing water and the microorganism and including a sidewall, the sidewall permits sunlight to pass therethrough to an interior of the housing, an artificial light source associated with the housing for emitting light into an interior of the housing, a sensor associated with the housing for sensing a quantity of sunlight passing through the sidewall and into the interior of the housing, and a controller electrically coupled to the sensor and the artificial light source, the controller is capable of activating the artificial light source when the sensor senses a less than desired quantity of sunlight passing into the interior of the housing.

In a further example, a container for cultivating a microorganism is provided and includes a housing for containing water and the microorganism, and a reflective element positioned outside of the housing for directing light toward an interior of the housing.

In still a further example, a method for cultivating microorganisms is provided and includes providing a container which contains water and includes a media at least partially positioned within the container, the media includes an elongated member and a plurality of loops extending from the elongated member, cultivating microorganisms within the container, removing the water and a first portion of the microorganisms from the container and leaving a second portion of the microorganisms on the media, refilling the container with water which does not contain the microorganisms, and cultivating microorganisms in the refilled container from the second portion of microorganisms that remained on the media.

In another example, a method for cultivating microorganisms is provided and includes providing a container which contains water and includes a media at least partially positioned within the container, cultivating microorganisms within the container, removing substantially all of the water and a first portion of the microorganisms from the container and leaving a second portion of the microorganisms on the media, refilling the container with water which does not contain the microorganisms, and cultivating microorganisms in the refilled container from the second portion of microorganisms that remained on the media.

In yet another example, a method for cultivating microorganisms is provided and includes providing a housing having a height dimension greater than a width dimension, positioning water into the container through a water inlet associated with the container, positioning a gas into the container through a gas inlet associated with the container, providing a plurality of media segments in the container, the plurality of media segments extend in a generally vertical direction and are spaced apart from one another, and cultivating microorganisms in the container, a first concentration of the microorganisms is supported by the plurality of media segments and a second concentration of microorganisms is suspended in the water, the first concentration of microorganisms is greater than the second concentration of microorganisms.

In still another example, a container for cultivating microorganisms is provided and includes a housing having a height dimension greater than a width dimension, the housing adapted to contain water and the microorganisms, a gas inlet associated with the housing for introducing gas into the container, a water inlet associated with the housing for introducing water into the container, and a plurality of media segments at least partially positioned within the housing, extending in a generally vertical direction, and spaced apart from one another, a first concentration of the microorganisms is supported by the plurality of media segments and a second concentration of microorganisms is suspended in the water, the first concentration of microorganisms is greater than the second concentration of microorganisms.

In a further example, a system for cultivating microorganisms is provided and includes a first container for containing water and cultivating microorganisms within the first container, a second container for containing water and cultivating microorganisms within the second container, and a conduit interconnecting the first container and the second container for carrying a gas out of the first container and into the second container.

In yet a further example, a container for cultivating a microorganism is provided and includes a housing for containing water and the microorganism, a first opening defined in the housing through which water is introduced into the housing at a first pressure, and a second opening defined in the housing through which water is introduced into the housing at a second pressure, the first pressure is greater than the second pressure.

In still a further example, a method for cultivating microorganisms is provided and includes providing a housing including a first opening and a second opening, cultivating microorganisms in the housing, introducing water into the housing through the first opening at a first pressure, and introducing water in the housing through the second opening at a second pressure, the first pressure is greater than the second pressure.

In another example, a system for cultivating microorganisms is provided and includes a container for containing water and the microorganisms, and a conduit for containing a fluid, the conduit is positioned to contact the water of the container, and a temperature of the fluid differs from a temperature of the water for changing the temperature of the water.

In yet another example, a method for cultivating microorganisms is provided and includes providing a container for containing water, positioning a frame at least partially within the container, coupling media to the frame, cultivating microorganisms on the media within the container, moving the frame and the media at a first speed, moving the frame and the media at a second speed different than the first speed, removing a portion of the water containing cultivated microorganisms from the container, and introducing additional water into the container to replace the removed water.

In still another example, a system for cultivating microorganisms is provided and includes a first container for containing water and for cultivating a first species of microorganism therein, a second container for containing water and for cultivating a second species of microorganism therein, the first species of microorganism is different than the second species of microorganism, a first conduit connected to the first container for carrying gas to the first container originating from a gas source, and a second conduit connected to the second container for carrying gas to the second container originating from the gas source.

In a further example, a system for cultivating microorganisms is provided and includes a first container for containing water and for cultivating microorganisms of a first species, a second container for containing water and for cultivating microorganism of the first species, a first conduit connected to the first container for carrying gas to the first container originating from a gas source, and a second conduit connected to the second container for carrying gas to the second container originating from the gas source, a first portion of the microorganisms cultivated is utilized to manufacture a first product and a second portion of the microorganisms cultivated is utilized to manufacture a second product.

In yet a further example, a system for cultivating microorganisms is provided and includes a first container for containing water and for cultivating a first species of microorganism therein, a second container for containing water and for cultivating a second species of microorganism therein, the first species of microorganism is different than the second species of microorganism, a first conduit connected to the first container for carrying gas to the first container, the gas originates from a gas source, and a second conduit connected to the second container for carrying gas to the second container, the gas originates from the gas source, and the first species of microorganism cultivated in the first container is utilized to manufacture a first product and the second species of microorganism cultivated in the second container is utilized to manufacture a second product.

In still a further example, a container for cultivating a microorganism is provided and includes a housing for containing water and the microorganism, the housing including a sidewall for permitting light to pass to an interior of the housing, and an ultraviolet inhibitor associated with the sidewall for inhibiting at least one wave length of light from passing through the sidewall.

In another example, a method for harvesting free oxygen during cultivation of microorganisms is provided and includes providing a container for containing water, the container including a frame and a media supported by the frame, introducing gas into the container, cultivating microorganisms within the container, moving the frame and media with a drive member to dislodge free oxygen from the media, the free oxygen is generated from cultivating the microorganisms, and removing the dislodged free oxygen from the container.

In yet another example, a system for cultivating microorganisms is provided and includes a first container for containing water and microorganisms, the first container includes a vertical dimension greater than a horizontal dimension, a second container for containing water and microorganisms, the second container includes a vertical dimension greater than a horizontal dimension, and the second container is positioned above the first container, a gas source providing a gas to the first and second containers for facilitating cultivation of the microorganisms within the first and second containers, and a water source providing the water to the first and second containers for facilitating cultivation of the microorganisms within the first and second containers.

In still another example, a container for cultivating microorganisms is provided and includes a housing for containing water and microorganisms, a frame at least partially positioned within the housing and including a first portion spaced apart from a second portion, a first media segment coupled to and extending between the first and second portions of the frame, a first portion of the microorganisms is supported by the first media segment, and a second media segment coupled to and extending between the first and second portions of the frame, a second portion of the microorganisms is supported by the second media segment, and the first media segment is spaced apart from the second media segment.

In a further example, a container for cultivating a microorganism is provided and includes a housing for containing water and the microorganism, a frame at least partially positioned within the housing, a drive member coupled to the frame to move the frame, a media supported by the frame and providing support for the microorganism during cultivation, and an artificial light source for providing light to an interior of the housing.

In yet a further example, a container for cultivating a microorganism is provided and includes a housing for containing water and the microorganism, a frame at least partially positioned within the housing, a media supported by the frame and providing support for the microorganism during cultivation, a first artificial light source for providing light to an interior of the housing, and a second artificial light source for providing light to the interior of the housing, the first and second artificial light sources are separate light sources.

In still a further example, a container for cultivating a microorganism is provided and includes a housing for containing water and the microorganism, a frame at least partially positioned within the housing, a media supported by the frame and providing support for the microorganism during cultivation, and an artificial light source disposed externally of the housing and for providing light to an interior of the housing, the artificial light source includes a member and a lighting element coupled to the member for emitting light, and the member is movable toward and away from the housing.

In another example, a container for cultivating a microorganism is provided and includes a housing for containing water and the microorganism, an at least partially opaque outer wall coupled to the housing and at least partially surround the housing, the at least partially opaque outer wall inhibits light from passing therethrough and into an interior of the housing, a frame at least partially positioned within the housing, a media supported by the frame and providing support for the microorganism during cultivation, and a light element coupled to the housing and the outer wall to transmit light from an exterior of the container to an interior of the housing.

In yet another example, a container for cultivating a microorganism is provided and includes an at least partially opaque housing for containing water and the microorganism, the at least partially opaque housing inhibits light from passing therethrough and into an interior of the housing, a frame at least partially positioned within the housing, a media supported by the frame and providing support for the microorganism during cultivation, and a light element coupled to the housing to transmit light from an exterior of the housing to an interior of the housing.

In still another example, a container for cultivating a microorganism is provided and includes a housing for containing water and the microorganism, and a member positioned outside of the housing and movable relative to the housing between a first position, in which the member at least partially surrounds a first portion of the housing, and a second position, in which the member at least partially surrounds a second portion of the housing, the first portion is greater than the second portion.

In a further example, a method for cultivating a microorganism is provided and includes providing a container for containing water and the microorganism, the container including a media at least partially positioned within the container, cultivating the microorganism on the media, removing at least a portion of the water from the container while retaining the microorganism on the media, and replacing at least a portion of the water removed back into the container.

In yet a further example, a container for cultivating a microorganism is provided and includes a housing for containing water and the microorganism, an inlet defined in the housing for permitting gas to enter the housing, a valve associated with the inlet which regulates the flow of gas into the housing, a pH sensor at least partially positioned within the housing to sense a pH level of water contained in the housing, and a controller electrically coupled to the valve and the pH sensor, the controller controls the valve dependent on a pH level of the water sensed by the pH sensor.

In still a further example, a container for cultivating a microorganism is provided and includes a housing for containing water and the microorganism, and a frame at least partially positioned within the housing and including a float device for providing buoyancy to the frame.

In another example, a system for cultivating algae is provided and includes a container with a media positioned therein providing a habitat in which the algae grows. The media is also capable of wiping the interior of the container to clear algae from the interior of the container. Also, the media may be loop cord media. The media may be suspended on a frame within the container and the frame may be rotatable. The frame may be rotated at a variety of speeds including a first slower speed, in which the media and algae supported on the media is rotated to control the time the algae is exposed to sunlight, and a second faster speed, in which the frame and the algae are rotated to dislodge the algae from the media. The system may include a flush system for assisting with removal of the algae from the media. For example, the flush system may include high pressure spraying apparatuses that spray the media and the algae supported thereon to dislodge the algae from the media. The frame and the media may be rotated during spraying. Further, the system may include an artificial light system to provide light other than direct sunlight to the container. For example, the artificial light system may re-direct natural sunlight toward the container or may provide artificial light. Further yet, the system may include an environmental control device for affecting the temperature of the container and the amount of light contacting the container.

### BRIEF DESCRIPTION OF THE DRAWINGS

FIG. 1 is a schematic of an exemplary microorganism cultivation system;

FIG. 2 is a schematic of another exemplary microorganism cultivation system;

FIG. 3 is a cross-sectional view taken along a longitudinal plane of a container of the systems shown in FIGS. 1 and 2;

FIG. 4 is an exploded view of the container shown in FIG. 3;

FIG. 5 is a top perspective view of a connector plate of the container shown in FIG. 3;

FIG. 6 is a front elevation view of a portion of an exemplary media for use in the container shown in FIG. 3;

FIG. 7 is a rear elevation view of the exemplary media shown in FIG. 6;

FIG. 8 is a front elevation view of the exemplary media shown in FIG. 6 with a support member;

FIG. 9 is an elevation view of another exemplary media for use in the container shown in FIG. 3;

FIG. 10 is a top view of the exemplary media shown in FIG. 9;

FIG. 11 is an elevation view of a further exemplary media for use in the container shown in FIG. 3;

FIG. 12 is a top view of the exemplary media shown in FIG. 11;

FIG. 13 is an elevation view of yet another exemplary media for use in the container shown in FIG. 3;

FIG. 14 is a top view of the exemplary media shown in FIG. 13;

FIG. 15 is an elevation view of still another exemplary media for use in the container shown in FIG. 3;

FIG. 16 is a top view of the exemplary media shown in FIG. 15;

FIG. 17 is an elevation view of still a further exemplary media for use in the container shown in FIG. 3;

FIG. 18 is a top view of the exemplary media shown in FIG. 17;

FIG. 18A is an elevation view of another exemplary media for use in the container shown in FIG. 3;

FIG. 18B is an elevation view of a further exemplary media for use in the container shown in FIG. 3;

FIG. 18C is an elevation view of yet another exemplary media for use in the container shown in FIG. 3;

FIG. 18D is an elevation view of still another exemplary media for use in the container shown in FIG. 3;

FIG. 18E is an elevation view of still a further exemplary media for use in the container shown in FIG. 3;

FIG. 19 is a top perspective view a portion of the connector plate of the container shown in FIG. 5 with media secured to the connector plate and a portion of the media schematically represented with lines;

FIG. 20 is a cross-sectional view of the container taken along line 20-20 in FIG. 3;

FIG. 21 is a cross-sectional view taken along line 21-21 in FIG. 20;

FIG. 22 is a top perspective view of a bushing of the container shown in FIG. 3;

FIG. 23 is a top view of an alternative embodiment of a bushing of the container shown in FIG. 3;

FIG. 24 is a top view of another alternative embodiment of a bushing of the container shown in FIG. 3;

FIG. 25 is a top perspective view of a container and an exemplary artificial light system;

FIG. 26 is a cross-sectional view taken along line 26-26 of FIG. 25;

FIG. 27 is a cross-sectional view taken along a longitudinal plane of a container and another exemplary artificial light system;

FIG. 28 is an enlarged view of a portion of the container and the artificial light system shown in FIG. 27;

FIG. 29 is an enlarged view of a portion of the container and the artificial light system shown in FIG. 27, shown with an alternative manner of wiping a portion of the artificial light system;

FIG. 30 is an elevation view of the container and the artificial light system shown in FIG. 27, shown with another alternative manner of wiping a portion of the artificial light system;

FIG. 31 is an enlarged view of a portion of the container and the artificial light system shown in FIG. 30;

FIG. 32 is a top perspective view of a portion of the container and a frame support device shown in FIG. 30;

FIG. 33 is a top view of the frame support device shown in FIG. 32;

FIG. 34 is an enlarged portion of FIG. 33;

FIG. 35 is a cross-sectional view of the frame support device taken along line 35-35 in FIG. 33;

FIG. 36 is an enlarged portion of FIG. 35;

FIG. 37 is a cross-sectional view taken along a longitudinal plane of the container and the frame support device shown in FIG. 32;

FIG. 38 is a partial cross-sectional view taken along a longitudinal plane of a container including a float device, shown in section, for supporting a frame of the container;

FIG. 39 is an elevation view of the float device shown in FIG. 38;

FIG. 40 is a top view of the float device shown in FIG. 38;

FIG. 41 is a top view of the float device shown in FIG. 38 including an exemplary lateral support plate;

FIG. 42 is a partial cross-sectional view taken along a longitudinal plane of another exemplary alternative container;

FIG. 43 is a top perspective view of a portion of the container and an exemplary alternative drive mechanism shown in FIG. 42;

FIG. 44 is a bottom perspective view of a portion of the container shown in FIG. 42;

FIG. 45 is a top perspective view of a portion of the container shown in FIG. 42;

FIG. 46 is a cross-sectional view taken along a longitudinal plane of a container and yet another exemplary artificial light system;

FIG. 47 is an enlarged view of a portion of the container and the artificial light system shown in FIG. 46;

FIG. 48 is a cross-sectional view taken along a longitudinal plane of a container and a further exemplary artificial light system;

FIG. 49 is a cross-sectional view taken along a longitudinal plane of a container, the container shown with a flushing system;

FIG. 50 is a top perspective view of a container with an exemplary temperature control system of the microorganism cultivation system;

FIG. 51 is a cross-sectional view taken along a longitudinal plane of a container, the container shown with another exemplary temperature control system of the microorganism cultivation system;

FIG. 52 is an elevation view of a container and a portion of an exemplary liquid management system;

FIG. 53 is an elevation view of an exemplary container, an exemplary environmental control device, and an exemplary support structure for supporting the container and the environmental control device in a vertical manner;

FIG. 54 is a cross-sectional view of a portion of the container and the environmental control device taken along line 54-54 in FIG. 53, the environmental control device is shown in a fully closed position;

FIG. 55 is a cross-sectional view of a portion of the container and the environmental control device similar to that shown in FIG. 54, the environmental control device is shown in a fully opened position;

FIG. 56 is a cross-sectional view of a portion of the container and the environmental control device similar to that shown in FIG. 54, the environmental control device is shown in a half-opened position;

FIG. 57 is a cross-sectional view of a portion of the container and the environmental control device similar to that shown in FIG. 54, the environmental control device is shown in another half-opened position;

FIG. 58 is a schematic view of a plurality of exemplary orientations of the environmental control device and an exemplary path of the Sun throughout a single day's time;

FIG. 59 is a schematic view of another exemplary environmental control device shown in a first position;

FIG. 60 is another schematic view of the environmental control device illustrated in FIG. 59, the environmental control device is shown in a second position or fully opened position;

FIG. 61 is yet another schematic view of the environmental control device illustrated in FIG. 59, the environmental control device is shown in a third position or a partially opened position;

FIG. 62 is a further schematic view of the environmental control device illustrated in FIG. 59, the environmental control device is shown in a fourth position or another partially opened position;

FIG. 63 is a top perspective view of a portion of an environmental control device including an exemplary artificial light system;

FIG. 64 is a cross-sectional view of the exemplary artificial light system taken along line 64-64 in FIG. 63;

FIG. 65 is a top perspective view of a portion of an environmental control device including another exemplary artificial light system;

FIG. 66 is a cross-sectional view of the exemplary artificial light system taken along line 66-66 in FIG. 65;

FIG. 66A is a top perspective view of another exemplary embodiment of a container;

FIG. 66B is a cross-sectional view taken along line 66B-66B in FIG. 66A;

FIG. 66C is a cross-sectional view similar to FIG. 66B showing yet another exemplary embodiment of a container;

FIG. 66D is a cross-sectional view similar to FIG. 66B showing still another exemplary embodiment of a container and an artificial light system;

FIG. 67 is an exemplary system diagram of the microorganism cultivation system showing, among other things, a relationship between a controller, a container, an artificial lighting system, and an environmental control device;

FIG. 68 is a flowchart showing an exemplary manner of operating the microorganism cultivation system;

FIG. 69 is a flowchart showing another exemplary manner of operating the microorganism cultivation system;

FIG. 70 is a flowchart showing yet another exemplary manner of operating the microorganism cultivation system;

FIG. 71 is a flowchart showing a further exemplary manner of operating the microorganism cultivation system;

FIG. 72 is a cross-sectional view taken along a plane perpendicular to a longitudinal extent of an exemplary alternative container, this exemplary container having a generally square shape;

FIG. 73 is a cross-sectional view taken along a plane perpendicular to a longitudinal extent of another exemplary alternative container, this exemplary container having a generally rectangular shape;

FIG. 74 is a cross-sectional view taken along a plane perpendicular to a longitudinal extent of yet another exemplary alternative container, this exemplary container having a generally triangular shape; and

FIG. 75 is a cross-sectional view taken along a plane perpendicular to a longitudinal extent of still another exemplary alternative container, this exemplary container having a generally oval shape.

Before any independent features and embodiments of the invention are explained in detail, it is to be understood that the invention is not limited in its application to the details of the construction and the arrangement of the components set forth in the following description or illustrated in the drawings. The invention is capable of other embodiments and of being practiced or of being carried out in various ways. Also, it is understood that the phraseology and terminology used herein is for the purpose of description and should not be regarded as limiting.

### DETAILED DESCRIPTION

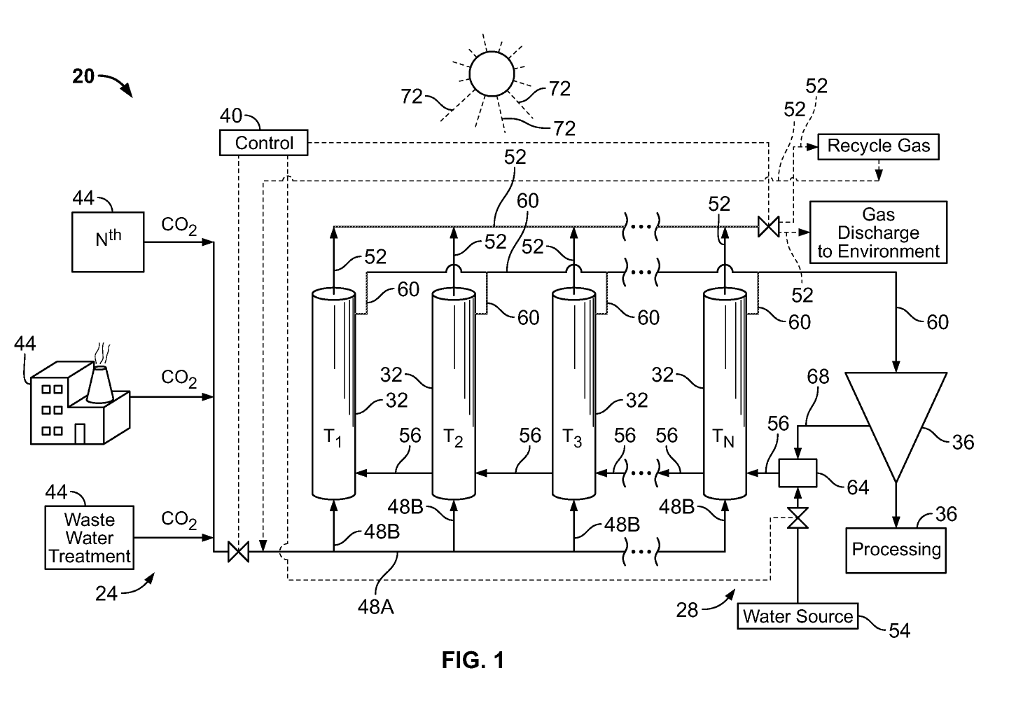

With reference to FIG. 1, an exemplary system 20 for cultivating microorganisms is illustrated. The system 20 is capable of cultivating a wide variety of types of microorganisms such as, for example, algae or microalgae. Microorganisms may be cultivated for a wide variety of reasons including, for example, comestible products, nutritional supplements, aquaculture, animal feed, nutraceuticals, pharmaceuticals, cosmetics, fertilizer, fuel production such as biofuels including, for example, biocrude, butanol, ethanol, aviation fuel, hydrogen, biogas, biodiesel, etc. Examples of microorganisms that may be cultivated include: P. tricornutum for producing polyunsaturated fatty acids for health and food supplements; Amphidinium sp. for producing Amphidinolides and amphidinins for anti-tumor agents; Alexandrium hiranoi for producing goniodomins for an antifungal agent; Oscillatoria agardhii for producing oscillapeptin, which is an elastase inhibitor, etc. While the present cultivation system 20 is capable of cultivating a wide variety of microorganisms for a wide variety of reasons and uses, the following description of the exemplary cultivation system 20 will be described as it relates to the cultivation of algae for fuel production.

Algae harvested from this exemplary system 20 undergoes processing to produce fuel such as, for example, biodiesel fuel, biodiesel fuel, jet fuel, and other products made from lipids extracted from microbes. As indicated above a wide variety of algae species, both fresh water and salt water species, may be used to in the system 20 to produce oil for fuel. Exemplary algae species include: Botryococcus barunii, Chaetoceros muelleri, Chlamydomonas rheinhardii, Chlorella vulgaris, Chlorella pyrenoidosa, Chlorococcum littorale, Dunaliella bioculata, Dunaliella salina, Dunaliella tertiolecta, Euglena gracilis, Haematococcus pluvialis, Isochrysis galbana, Nannochloropsis oculata, Navicula saprophila, Neochloris oleoabundans, Porphyridium cruentum, P. Tricornutum, Prymnesium parvum, Scenedes Musdimorphus, Scenedesmus dimorphus, Scenedesmus obliquus, Scenedesmus quadricauda, Spirulina maxima, Spirulina platensis, Spirogyra sp., Synechoccus sp., Tetraselmis maculata, Tetraselmis suecica, etc. For these and other algae species, high oil content and/or ability to mitigate carbon dioxide are desirable in order to produce large quantities of fuel and/or consume large quantities of carbon dioxide.

Different types of algae require different types of environmental conditions in order to efficiently grow. Most types of algae must be cultivated in water, either fresh water or salt water. Other required conditions are dependent on the type of algae. For example, some types of algae may be cultivated solely with the addition of light, carbon dioxide, and minimal amounts of minerals to the water. Such minerals may include, for example, nitrogen and phosphorus. Other types of algae may require other types of additives for proper cultivation.

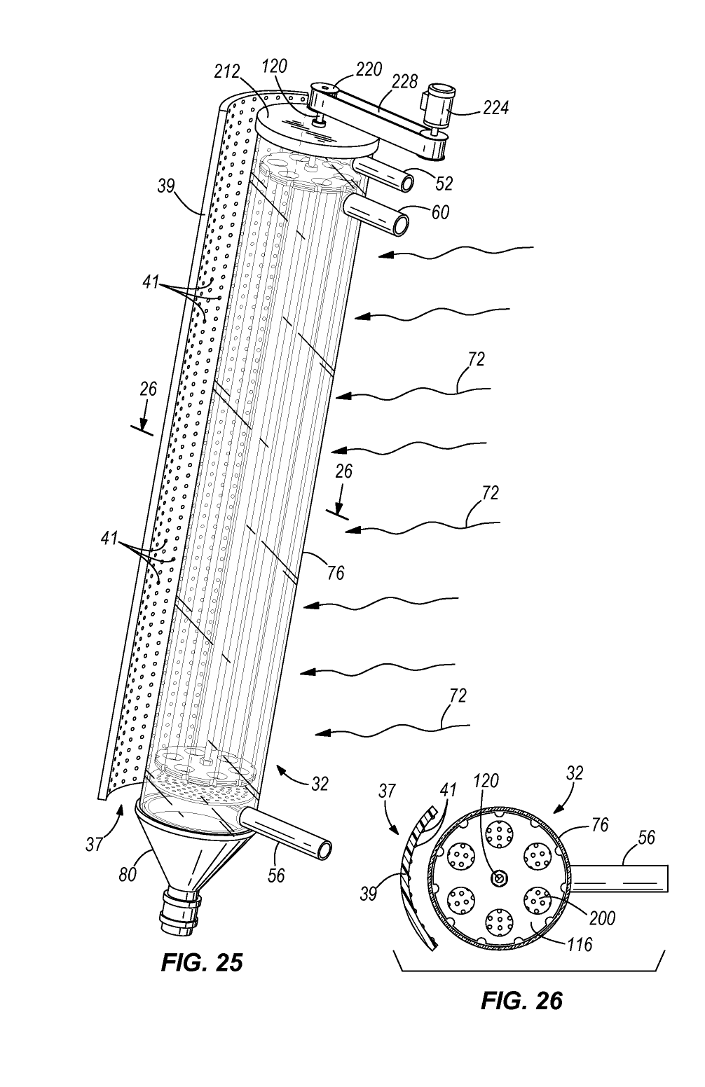

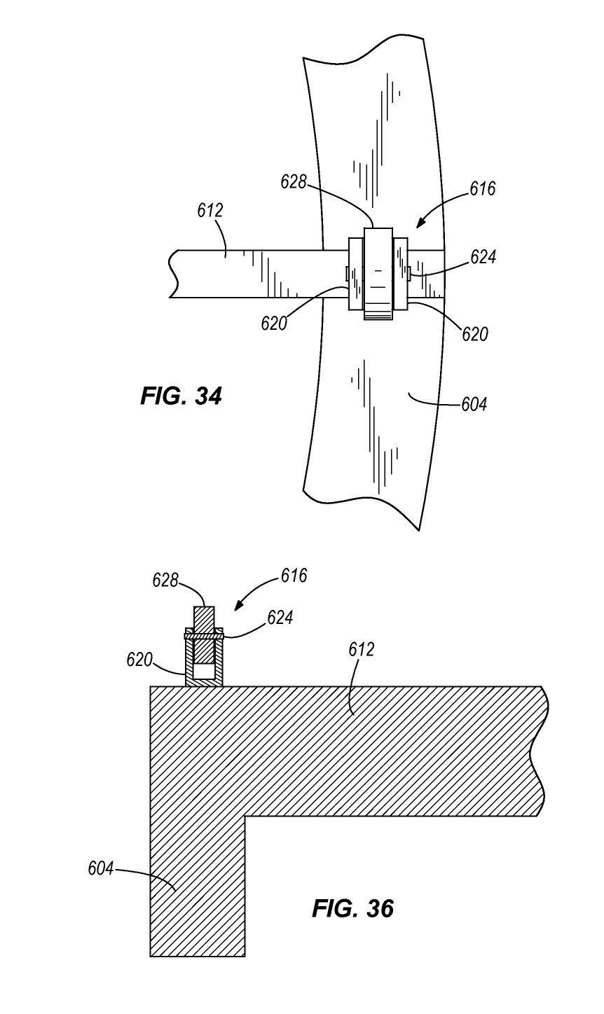

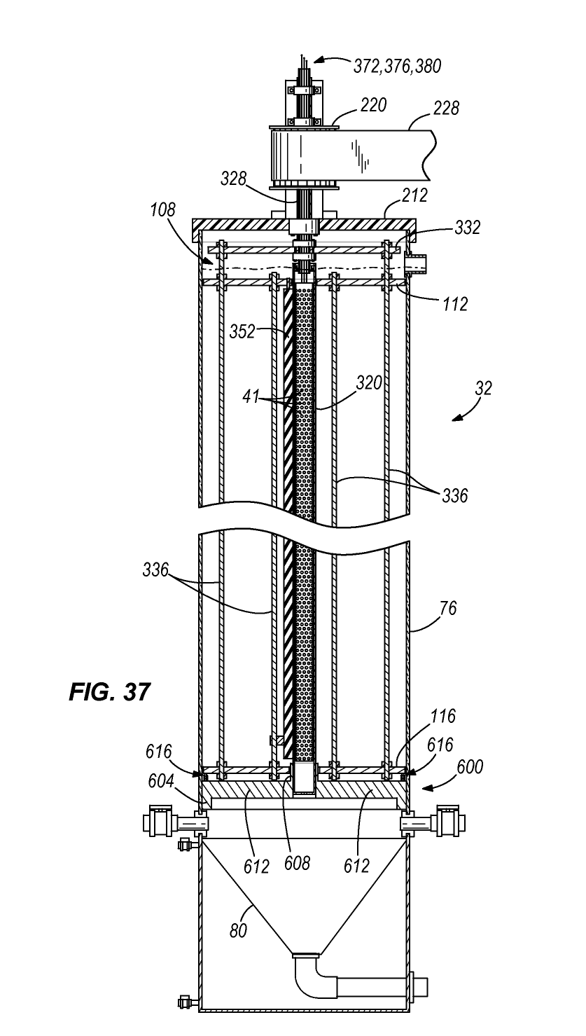

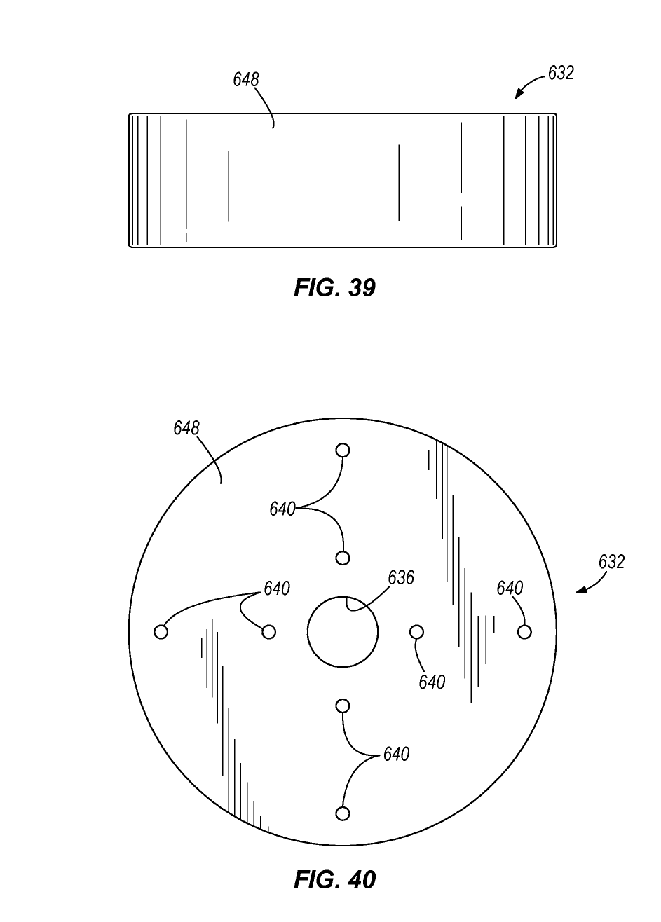

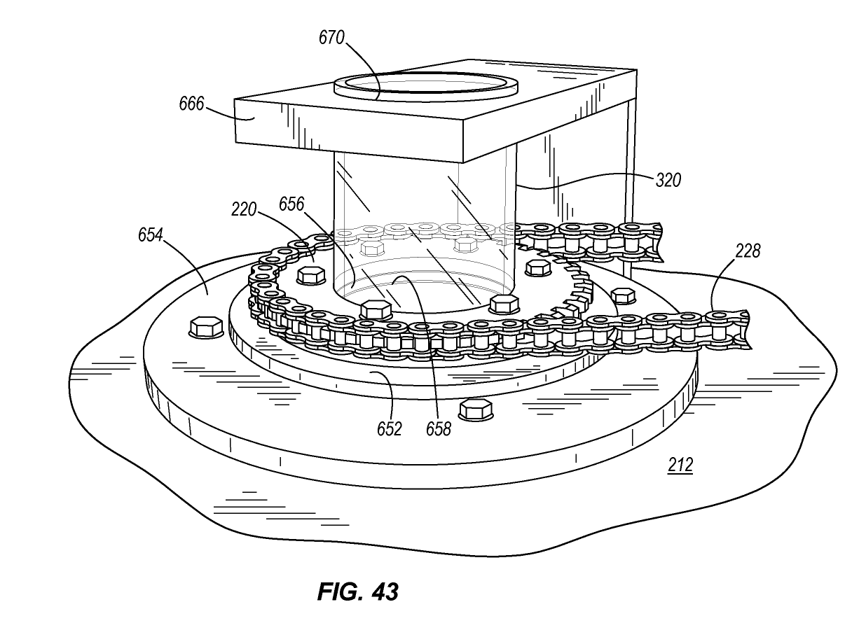

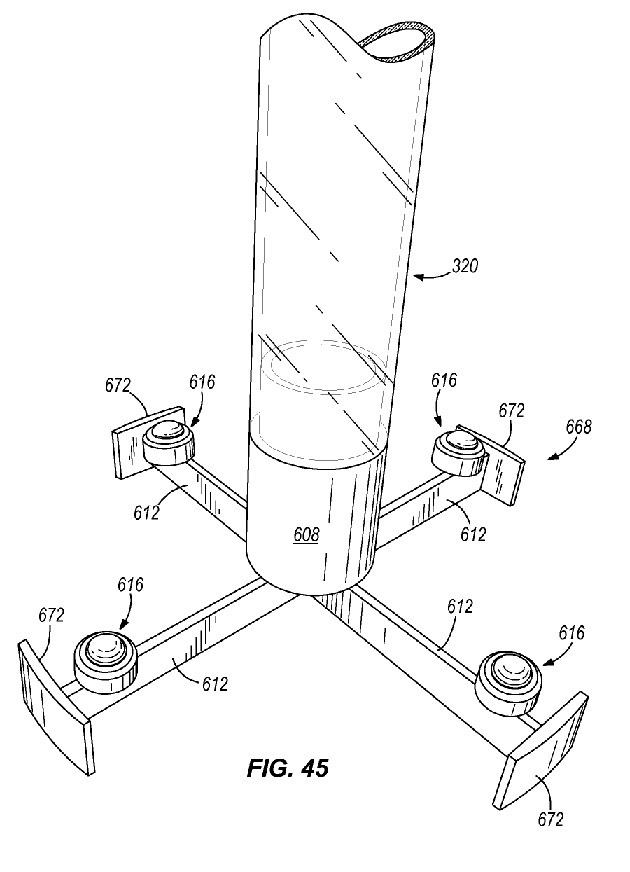

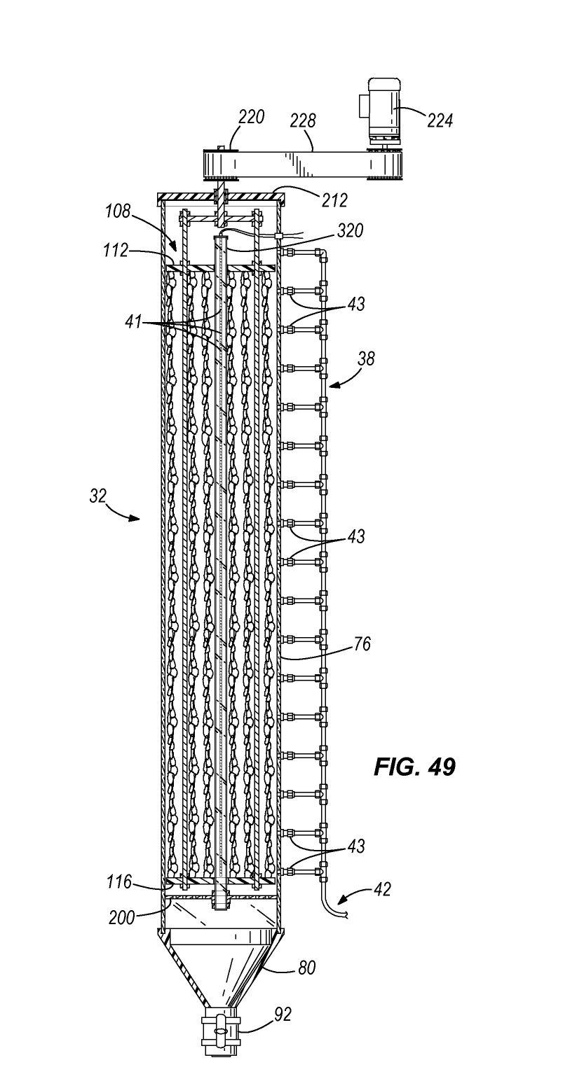

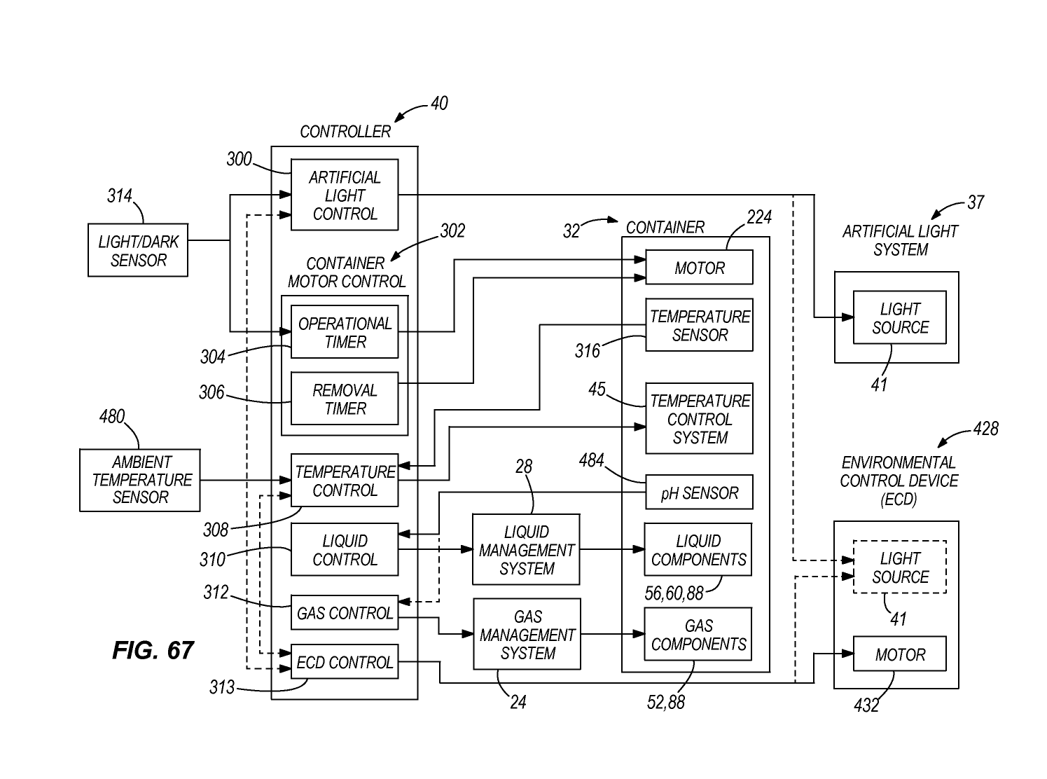

With continued reference to FIG. 1, the system 20 includes a gas management system 24, a liquid management system 28, a plurality of containers 32, algae collection processing equipment 36, an artificial light system 37 (see FIGS. 25-48 and 63-66), a clean-in-place or flushing system 38 (see FIG. 49), and a programmable logic controller 40 (see FIG. 67). The gas management system 24 includes at least one carbon dioxide source 44, which can be one or more of a wide variety of sources. For example, the carbon dioxide source 44 may be emissions generated from an industrial facility, a manufacturing facility, fuel powered equipment, a byproduct generated from a waste water treatment facility, or a pressurized carbon dioxide canister, etc. Exemplary industrial and manufacturing facilities may include, for example, power plants, ethanol plants, cement processors, coal burning plants, etc. It is preferred that the gas from the carbon dioxide source 44 does not contain toxic levels of sulfur dioxide or other toxic gases and compounds, such as heavy metals, that may inhibit microbial growth. If the gas exhausted from a source includes sulfur dioxide or other toxic gases, it is preferable that the gas be scrubbed or purified prior to introduction into the containers 32. The gas management system 24 introduces carbon dioxide to the containers 32 in a feed stream. In some exemplary embodiments, the feed stream may comprise between about 10% and about 12% of carbon dioxide by volume. Alternatively, the feed stream may comprise other percentages of carbon dioxide by volume and still be within the spirit and scope of the present invention.

In instances where the carbon dioxide originates from industrial emissions, machinery emissions, or byproducts from waste water treatment facilities, the system 20 is recycling carbon dioxide for a useful purpose rather than allowing the carbon dioxide to release into the atmosphere. The carbon dioxide source 44 for the system 20 can be a single source 44, a plurality of similar sources 44 (e.g., a plurality of industrial facilities), or a plurality of different sources 44 (e.g., an industrial facility and a waste water treatment facility). The gas management system 24 includes a network of pipes 48 that delivers the carbon dioxide derived from the carbon dioxide source(s) 44 to each of the containers 32. In some embodiments, prior to the gas management system 24 introducing the carbon dioxide into the containers 32, the emissions from which the carbon dioxide originates may be passed through a cooling spray tower for cooling and introduced into solution. In the illustrated exemplary embodiment of FIG. 1, the containers 32 are connected in parallel via the pipes 48. As represented in the illustrated exemplary embodiment, the network of pipes 48 includes a main inlet line 48A and a plurality of secondary inlet branches 48B, which extend from the main inlet line 48A and route the carbon dioxide from the main inlet line 48A to each of the plurality of containers 32. The secondary inlet branches 48B are connected to the bottom of the containers 32 and release the carbon dioxide into the interior of container 32 which is generally filled with water. When introduced into the containers 32, the carbon dioxide assumes the form of bubbles in the water and ascends through the water to the top of the containers 32. In some examples, the pressure range contemplated for the introduction of the carbon dioxide is about 25-50 pounds per square inch (psi). The gas management system 24 may include a gas sparger, diffuser, bubble distributor, water saturated gas injection, or other device located at the bottom of the containers 32 to introduce the carbon dioxide bubbles into the containers 32 and more evenly distribute the carbon dioxide throughout the container. Additionally, other gas spargers, diffusers, bubble distributors, or other devices may be incrementally disposed within and along the height of containers 32 to introduce carbon dioxide bubbles into the containers 32 at multiple height locations. The carbon dioxide gas that is introduced into container 32 is, at least in part, consumed by algae contained within container 32 in the growth and cultivation process. As a result, less carbon dioxide is discharged from container 32 than is introduced into container 32. In some embodiments, the gas management system 24 may include, where necessary, gas pre-filtering, cooling, and toxic gas scrubbing elements.

The gas management system 24 further includes gas discharge pipes 52. As described above, carbon dioxide that is not consumed by algae within the container 32 migrates up the container 32 and accumulates in the upper region of each of the containers 32. The consumption of carbon dioxide by the algae occurs with the algae undergoing the photosynthesis process which is necessary for the cultivation of the algae. A byproduct of the photosynthesis process is the production of oxygen by the algae which is released into the water of the container 32 and may settle or nucleate on the media 110 and algae, or may rise and accumulate at the top region of the container 32. High oxygen levels in the water and container 32 may cause oxygen inhibition, which inhibits the algae from consuming carbon dioxide and ultimately inhibits the photosynthesis process. Accordingly, it is desirable to exhaust oxygen from the container 32.

The accumulated carbon dioxide and oxygen can be exhausted from the containers 32 in a variety of manners including, for example, to the environment, back into the main gas line for recycling, to an industrial facility as fuel for combustions processes such as powering the industrial facility, or to further processes where additional carbon dioxide can be extracted.

It should be understood that the illustrated exemplary system 20 is efficient at scrubbing or consuming the carbon dioxide present in the incoming gas. Accordingly, the exhausted gas has relatively low amounts of carbon dioxide and can be safely exhausted to the environment. Alternatively, the exhausted gas can be rerouted to the main gas line where the exhausted gas mixes with the gas present in the main gas line for reintroduction into the containers 32. Further, a portion of the exhausted gas can be exhausted to the environment and a portion of the gas can be reintroduced into the main gas line or sent for further processing.

The liquid management system 28 comprises a water source 54, a network of pipes including water inlet pipes 56 that provide water to the containers 32, water outlet pipes 60 that exhaust water and algae from the containers 32, and at least one pump 64. The pump 64 controls the amount and rate at which water is introduced into the containers 32 and exhausted from the containers 32. In some embodiments, the liquid management system 28 may include two pumps, one for controlling the introduction of water into the containers 32 and one for controlling exhaustion of water and algae from the containers 32. The liquid management system 28 may also comprise water reclamation pipes 68 that reintroduce the used water, which was previously exhausted from the containers 32 and filtered to remove the algae, back into the water inlet pipes 56. This recycling of the water within the system 20 decreases the amount of new water required to cultivate algae and may provide algae seeding for subsequent batches of algae cultivation.

The plurality of containers 32 are utilized to cultivate algae therein. The containers 32 are sealed-off from the surrounding environment and the internal environment of the containers 32 is controlled by the controller 40 via the gas and liquid management systems 24, 28 among other components described in greater detail below. With reference to FIG. 67, the controller 40 includes an artificial light control 300, a motor control 302 having an operational timer 304 and a removal timer 306, a temperature control 308, a liquid control 310, a gas control 312, and an environmental control device (ECD) control 313. Operation of the controller 40 as it relates to the components of the microorganism cultivating system 20 will be described in greater detail below. In an exemplary embodiment, the controller 40 may be an Allen Bradley CompactLogix programmable logic controller (PLC). Alternatively, the controller 40 may be other types of devices for controlling the system 20 in the manner described herein.

In some embodiments, the containers 32 are oriented in a vertical manner and may be arranged in a relatively tightly packed side-by-side array in order to efficiently utilize space, with for example, containers ranging 3 inches to 6+ feet in width or diameter, and 6 to 30+ feet in height. For example, a single acre of land may include about 2000 to 2200 containers having a 24-inch diameter. In other embodiments, the containers are stacked one above another to provide an even more efficient use of space. In such embodiments where the containers are stacked, gas introduced into a bottom container may ascend through the bottom container and, upon reaching the top of the bottom container, may be routed to a bottom of a container positioned above the bottom container. In this manner, the gas may be routed through several containers in order to effectively utilize the gas.

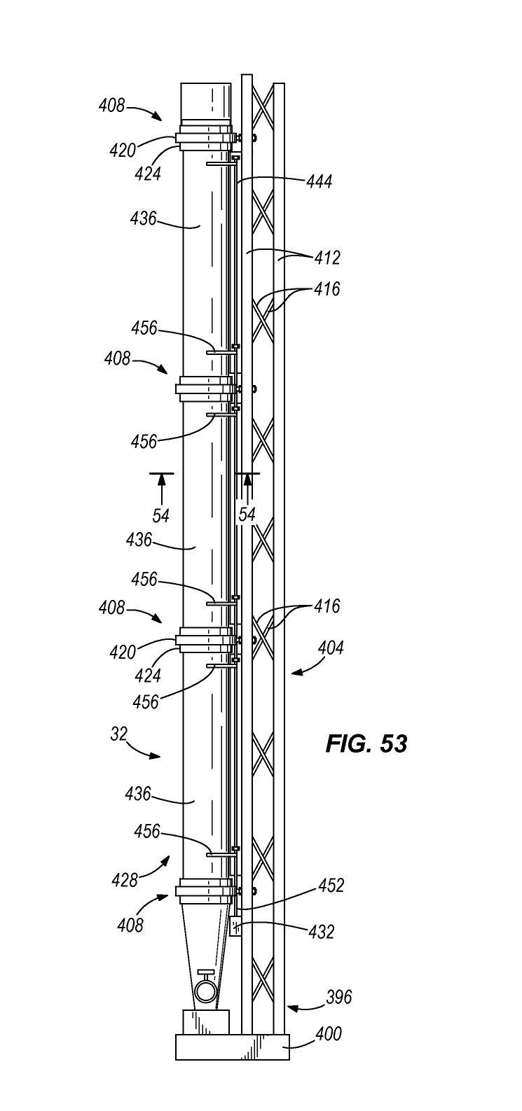

The containers 32 may be vertically supported in a variety of different manners. One exemplary manner of vertically supporting the containers 32 is illustrated in FIG. 53 and is described in greater detail below. This illustrated example is only one of many exemplary manners of supporting the containers 32 and is not intended to be limiting. Other manners of supporting the containers 32 are contemplated and are within the spirit and scope of the present invention.

Sunlight 72 is an important ingredient of the photosynthesis process utilized in the algae cultivation system 20. The containers 32 are arranged to receive direct sunlight 72 to facilitate the photosynthesis process. Photosynthesis in combination with the carbon dioxide introduced into the containers 32 facilitates cultivation of the algae therein.

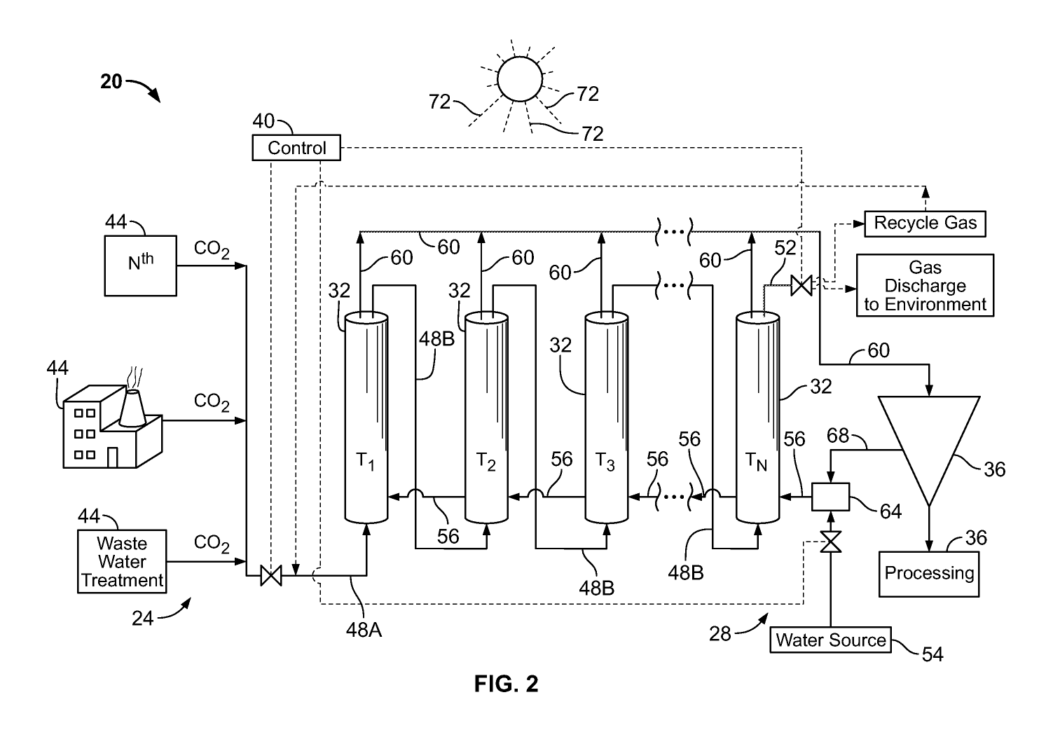

Referring now to FIG. 2, another exemplary system 20 for cultivating algae is illustrated and has many similarities to the system 20 illustrated in FIG. 1, particularly with respect to the plurality of containers 32, the liquid management system 28, and the controller 40. Similar components between embodiments illustrated in FIGS. 1 and 2 include similar reference numbers. In the exemplary embodiment illustrated in FIG. 2, the containers 32 are connected in-series by way of the gas management system 24 through the network of pipes 48, which is in contrast with the embodiment illustrated in FIG. 1 where the containers 32 are connected in-parallel. When connected in-series, the gas management system 24 includes a main inlet line 48A that introduces gas into the bottom of a first container 32 and includes a plurality of serial secondary inlet branches 48B that transport the exhausted gas from one container 32 to the bottom of the next container 32. After the last container 32, the gas is exhausted from the container 32 through the gas discharge pipe 52 to any one or more of the environment, reintroduced into the main gas line, or delivered for further processing.

As indicated above, the gas source 44 may be an industrial or manufacturing facility, which may exhaust gas having elements detrimental to cultivation of one algae species, but beneficial for cultivation of a second algae species. In such instances, containers 32 may be connected in-series via the gas management system 24, as described above and illustrated in FIG. 2, to accommodate such exhaust gas. For example, a first container 32 may contain a first algae species that prospers in the presence of a particular element of the exhaust gas and a second container 32 may contain a second algae species that does not prosper in the presence of the particular element of the exhaust gas. With the first and second containers 32 connected in-series, the exhaust gas enters the first container 32 and the first algae species substantially consumes the particular element of the exhaust gas for cultivation purposes. Then, the resulting gas from the first container 32, which substantially lacks the particular element, is transported via the gas management system 24 to the second container 32 where the second algae species consumes the resulting gas for cultivation purposes. Since the resulting gas is substantially deficient of the particular element, cultivation of the second algae species is not inhibited by the gas. In other words, the first container 32 acts as a filter to remove or consume a particular element or elements present in the exhaust gas that may be detrimental to other species of algae present in subsequent containers 32.

It should be understood that the plurality of containers 32 can be connected to one another in a combination of both parallel and serial manners and the gas management system 24 can be appropriately configured to route gas to the containers 32 in both serially and parallel manners.

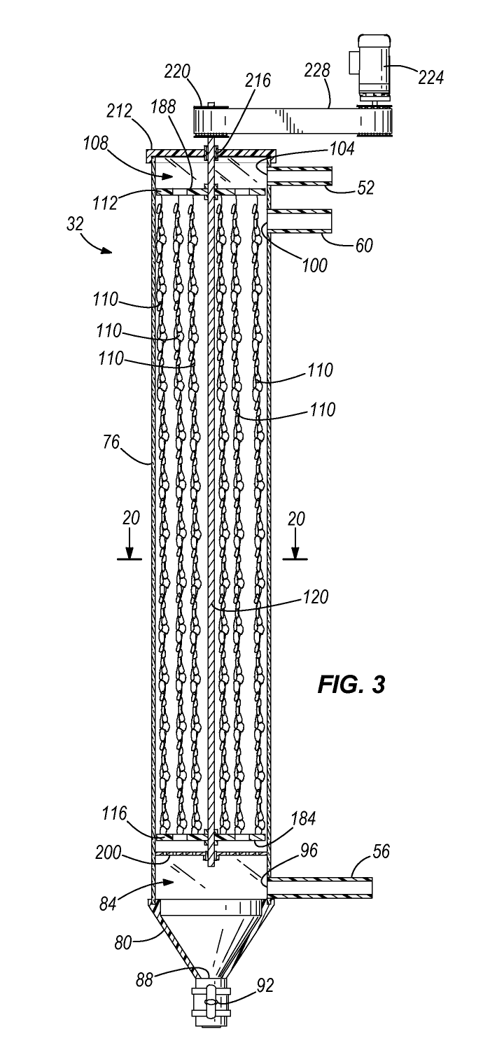

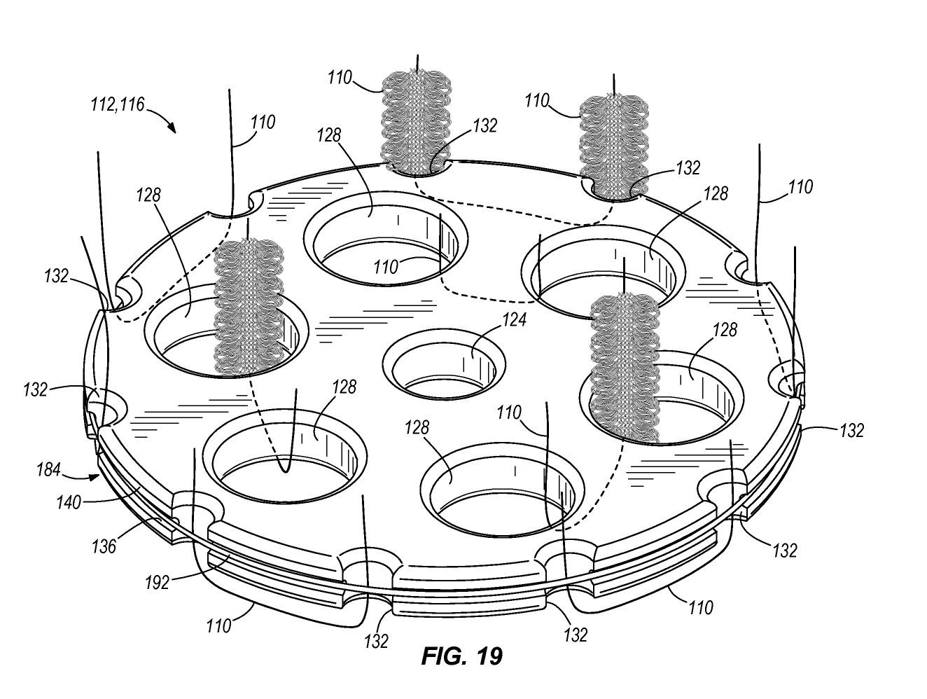

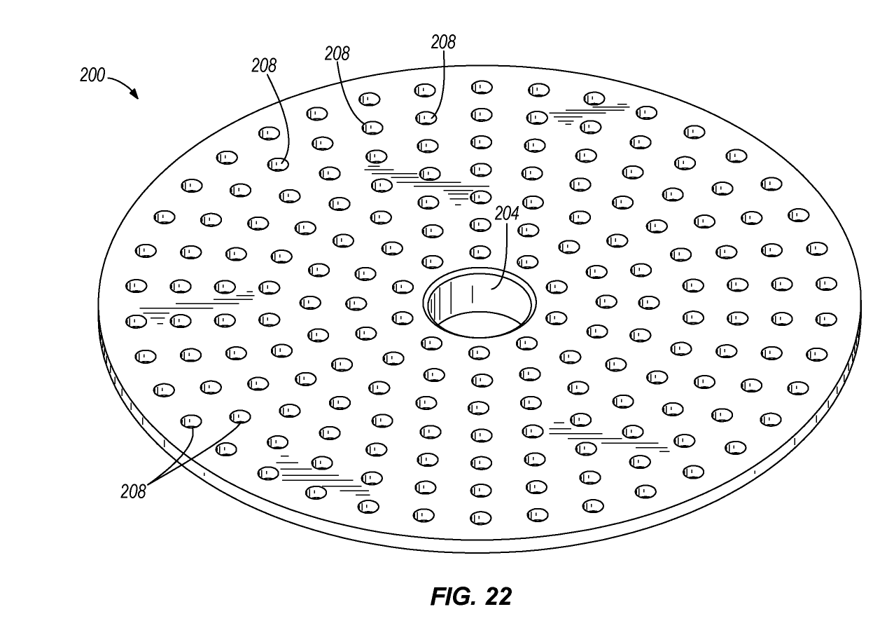

With reference to FIGS. 3-22, the plurality of containers 32 will be described in greater detail. In this example, the plurality of containers 32 are all substantially identical and, therefore, only a single container 32 is illustrated and described herein. The illustrated and described container 32 is only an exemplary embodiment of the container 32. The container 32 is capable of having a different configuration and capable of including different components. The illustrated container 32 and accompanying description is not meant to be limiting.

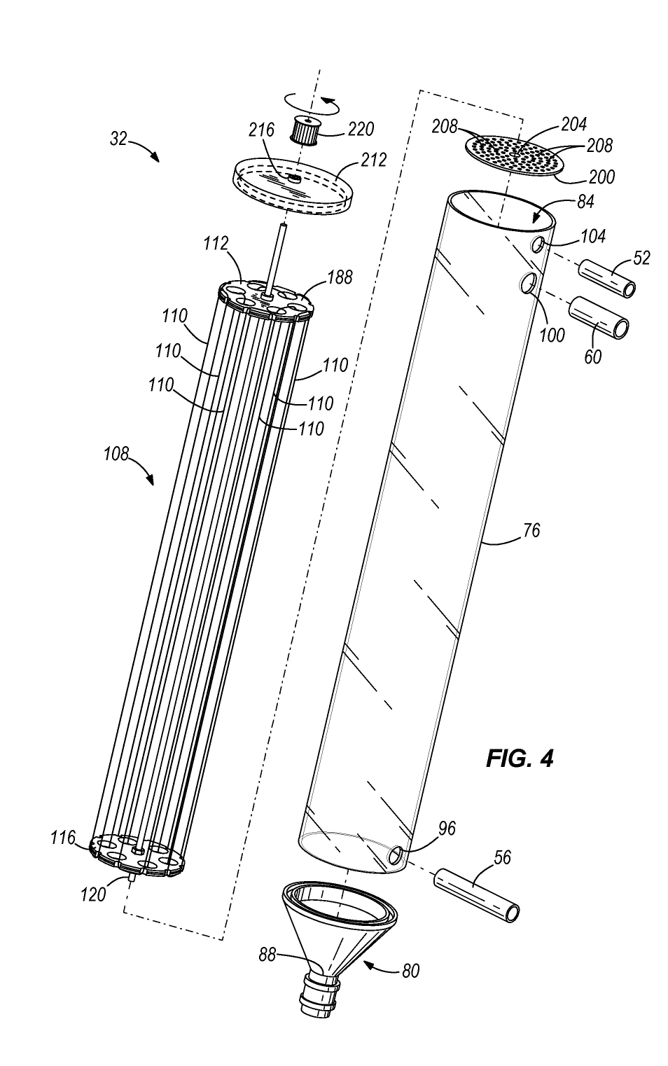

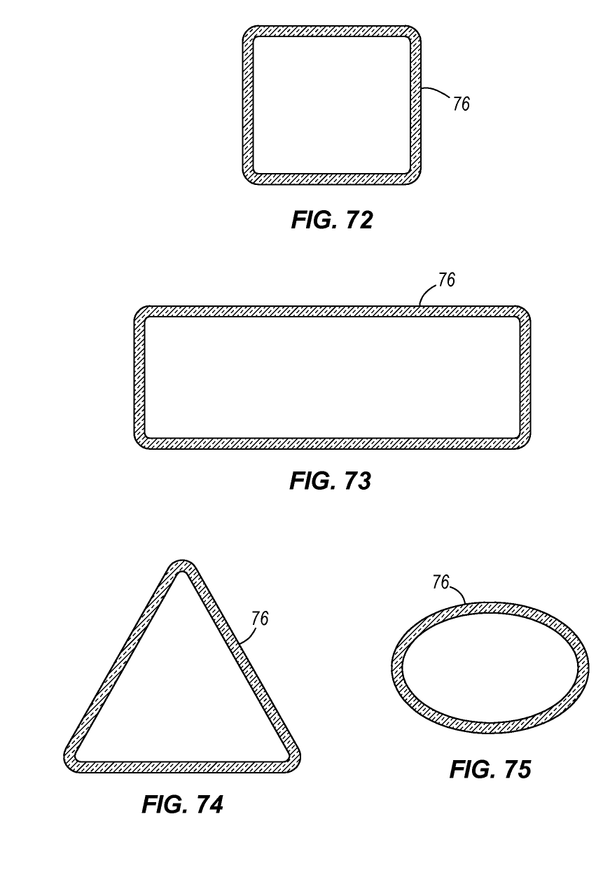

With particular reference to FIGS. 3 and 4, the illustrated exemplary container 32 includes a cylindrical housing 76 and a frusto-conical base 80. Alternatively, the housing 76 can have different shapes, some of which will be described in greater detail below with reference to FIGS. 72-75. In the illustrated exemplary embodiment, the housing 76 is completely clear or transparent, thereby allowing a significant amount of sunlight 72 to penetrate through the housing 76, into the cavity 84, and contact the algae contained within the container 32. In some embodiments, the housing 76 is translucent to allow penetration of some sunlight 72 through the housing 76 and into the cavity 84. In other embodiments, the housing 76 may be coated with infrared inhibitors, Ultraviolet blockers, or other filtering coatings to inhibit heat, ultraviolet rays, and/or particular wavelengths of light from penetrating through the housing 76 and into the container 32. The housing 76 can be made of a variety of materials including, for example, plastic (such as polycarbonate), glass, and any other material that allows penetration of sunlight 72 through the housing 76. One of the many possible materials or products from which the housing 76 may be made is the translucent aquaculture tanks manufactured by Kalwall Corporation of Manchester, N.H.

In some embodiments, the housing 76 may be made of a material that does not readily form a desired shape of the housing 76 under normal circumstances such as, for example, cylindrical. In such embodiments, the housing 76 may want to form an oval cross-sectional shape rather than a substantially round cross-sectional shape. To assist the housing 76 with forming the desired shape, additional components may be required. For example, a pair of support rings may be disposed within and secured to the housing 76, one near the top and one near the bottom. These support rings are substantially circular in shape and assist with forming the housing 76 into the cylindrical shape. In addition, other components of the container 32 may assist the housing 76 with forming the cylindrical shape such as, for example, upper and lower connector plates 112, 116, a bushing 200, and a cover 212 (all of which are described in greater detail below). Example of materials that may be used to make the container housing 76 may include polycarbonate, acrylic, LEXAN® (a highly durable polycarbonate resin thermoplastic), fiber re-enforced plastic (FRP), laminated composite material (glass plastic laminations), glass, etc. Such materials may be formed in a sheet and rolled into a substantially cylindrical shape such that edges of the sheet engage each other and are bonded, welded, or otherwise secured together in an air and water tight manner. Such a sheet may not form a perfectly cylindrical shape when at rest, thereby requiring the assistance of such components described above to form the desired shape. Also, such materials may be formed in the desired cylindrical shape.

The base 80 includes an opening 88 through which carbon dioxide gas is injected from the gas management system 24 into the container 32. A gas valve 92 (see FIG. 3) is coupled between the gas management system 24 and the base 80 of the container 32 to selectively prevent or allow the flow of gas into the container 32. In some embodiments, the gas valve 92 is electronically coupled to the controller 40 and the controller 40 determines when the gas valve 92 is opened and closed. In other embodiments, the gas valve 92 is manually manipulated by a user and the user determines when the gas valve 92 is opened and closed.

With continued reference to FIGS. 3 and 4, the housing 76 also includes a water inlet 96 in fluid communication with the liquid management system 28 to facilitate the flow of water into the container 32. In the illustrated exemplary embodiment, the water inlet 96 is disposed in the housing 76 near a bottom of the housing 76. Alternatively, the water inlet 96 may be disposed closer to or further from the bottom. In the illustrated exemplary embodiment, the housing 76 includes a single water inlet 96. Alternatively, the housing 76 may include a plurality of water inlets 96 to facilitate injection of water into the container 32 from a plurality of locations. In some embodiments, the water inlet 96 is defined in the base 80 of the container 32 rather than the housing 76.

The housing 76 further includes a plurality of water outlets 100 in fluid communication with the liquid management system 28 to facilitate the flow of water out of the container 32. In the illustrated exemplary embodiment, the water outlets 100 are disposed near a top of the housing 76. Alternatively, the water outlets 100 may be disposed closer to or further from the top of the housing 76. In some embodiments, the water outlets 100 are defined in the base 80 of the container 32. While the illustrated exemplary embodiment of the housing 76 includes two water outlets 100, the housing 76 is alternatively capable of including a single water outlet 100 to facilitate the flow of water from the container 32. In other embodiments, the opening 88 could be used as an outlet or drain for the water within the container 32.

The housing 76 also includes a gas outlet 104 in fluid communication with the gas management system 24 to facilitate the flow of gas out of the container 32. During operation, gas accumulates, as discussed above, at the top of the housing 76 and, accordingly, the gas outlet 104 is disposed near a top of the housing 76 in order to accommodate the gas build-up. While the illustrated exemplary embodiment of the housing 76 includes a single gas outlet 104, the housing 76 is alternatively capable of including a plurality of gas outlets 104 to facilitate the flow of gas out of the container 32.

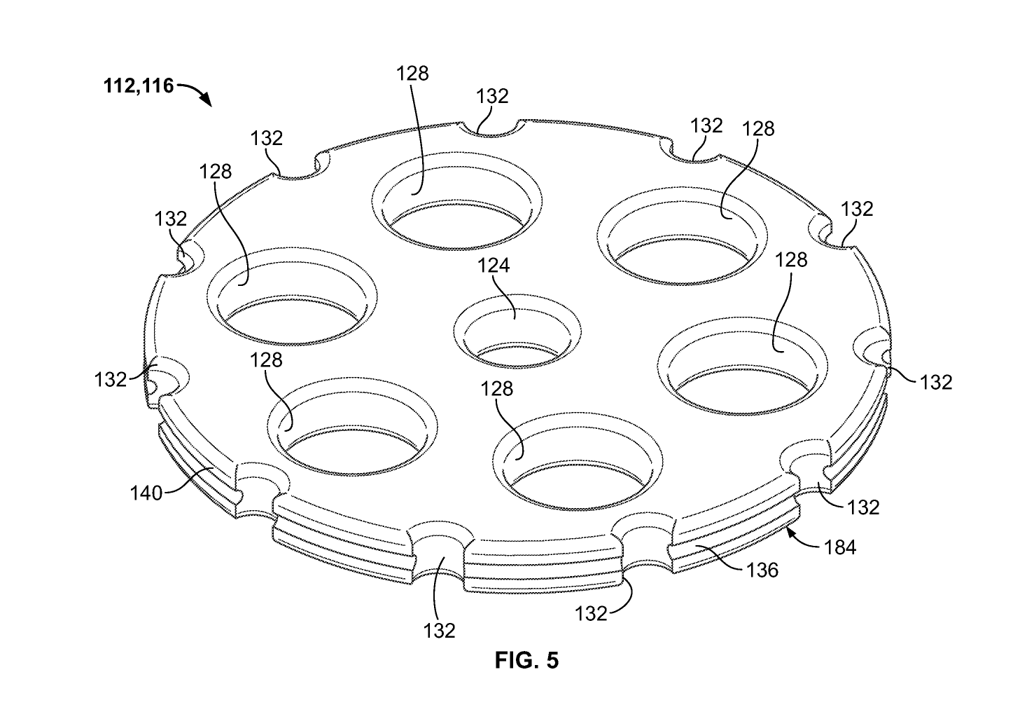

With continued reference to FIGS. 3 and 4, the container 32 further includes a media frame 108 positioned in the housing cavity 84 and for supporting media 110 thereon. As used herein, the term “media” means a structural element providing at least one surface for supporting and facilitating cultivation of microorganisms. The frame 108 includes an upper connector plate 112, a lower connector plate 116, and a shaft 120. In this example, the upper and lower connector plates 112, 116 are substantially identical. Referring now to FIG. 5, the upper and lower connector plates 112, 116 are substantially circular in shape and include a central aperture 124 for receiving the shaft 120. In some embodiments, the central aperture 124 is appropriately sized to receive the shaft 120 and provide a press-fit or resistance-fit connection between the shaft 120 and the connector plates 112, 116. In such an embodiment, no additional fastening or bonding is required to secure the connector plates 112, 116 to the shaft 120. In other embodiments, the shaft 120 is fastened to the upper and lower connector plates 112, 116. The shaft 120 can be fastened to the connector plates 112, 116 in a variety of manners. For example, the shaft 120 can include threads thereon and the interior surface of the central apertures 124 of the connector plates 112, 116 can include complimentary threads, thereby facilitating threading of the connector plates 112, 116 onto the shaft 120. Also, for example, the shaft 120 may include threads thereon, the shaft 120 may be inserted through the central apertures 124 of the connector plates 112, 116, and nuts can be threaded onto the shaft 120 both above and below each of the connector plates 112, 116, thereby compressing the connector plates 112, 116 between the nuts and securing the connector plates 112, 116 to the shaft 120. In yet other embodiments, the connector plates 112, 116 can be bonded to the shaft 120 in a variety of manners such as, for example, welding, brazing, adhering, etc. No matter the manner in which the connector plates 112, 116 are secured to the shaft 120, a rigid connection between the connector plates 112, 116 and the shaft 120 is desired to inhibit movement of the connector plates 112, 116 relative to the shaft 120.

It should be understood that the frame 108 may include other devices in place of the connector plates 112, 116 such as, for example, metal or plastic wire screens, metal or plastic wire matrices, etc. In such alternatives, the media 110 may be looped through and around openings present in the screens or matrices or may be affixed to the screens and matrices with fasteners such as, for example, hog rings.

With continued reference to FIG. 5, the upper and lower connector plates 112, 116 include a plurality of apertures 128 defined therethrough, a plurality of recesses 132 defined in a periphery of the connector plates 112, 116, and a slot 136 defined in an outer peripheral edge 140 of the connector plates 112, 116. All of the apertures 128, recesses 132, and the slot 136 are used to secure the media 110 to the connector plates 112, 116. In the illustrated exemplary embodiment, the connector plates 112, 116 are connected to the shaft 120 such that the apertures 128 and recesses 132 of the connector plate 112 vertically align with corresponding apertures 128 and recesses 132 of the connector plate 116. The configuration and size of the apertures 128 and recesses 132 in the illustrated exemplary embodiment of the connector plates 112, 116 are for exemplary illustrative purposes only and are not meant to be limiting. The connector plates 112, 116 are capable of having different configurations and sizes of apertures 128 and recesses 132. In some examples, the configuration and size of the apertures 128 and recesses 132 is dependent upon the type of algae being cultivated in the container 32. Algae that has lush growth requires greater spacing between strands of media 110, whereas algae having less lush growth may have strands of media 110 more closely packed. For example, algae species C. Vulgaris and Botryococcus barunii grow very lushly and the spacing of the individual media strands 110 may be about 1.5 inches on center. Also, for example, algae species Phaeodactylum tricornutum may not exhibit as lush of growth as C. Vulgaris or Botryococcus barunii and, accordingly, spacing of the individual media strands 110 is decreased to about 1.0 inch on center. Additionally, for example, the spacing of the individual media strands 110 is about 2+ inches on center for the algae species B. Braunii. It should be understood that the spacing of the individual media strands 110 may be established dependent on the species of algae being cultivated and the exemplary spacing described herein are for illustrative purposes and are not intended to be limiting. Connection of the media 110 to the connector plates 112, 116 will be described in greater detail below.

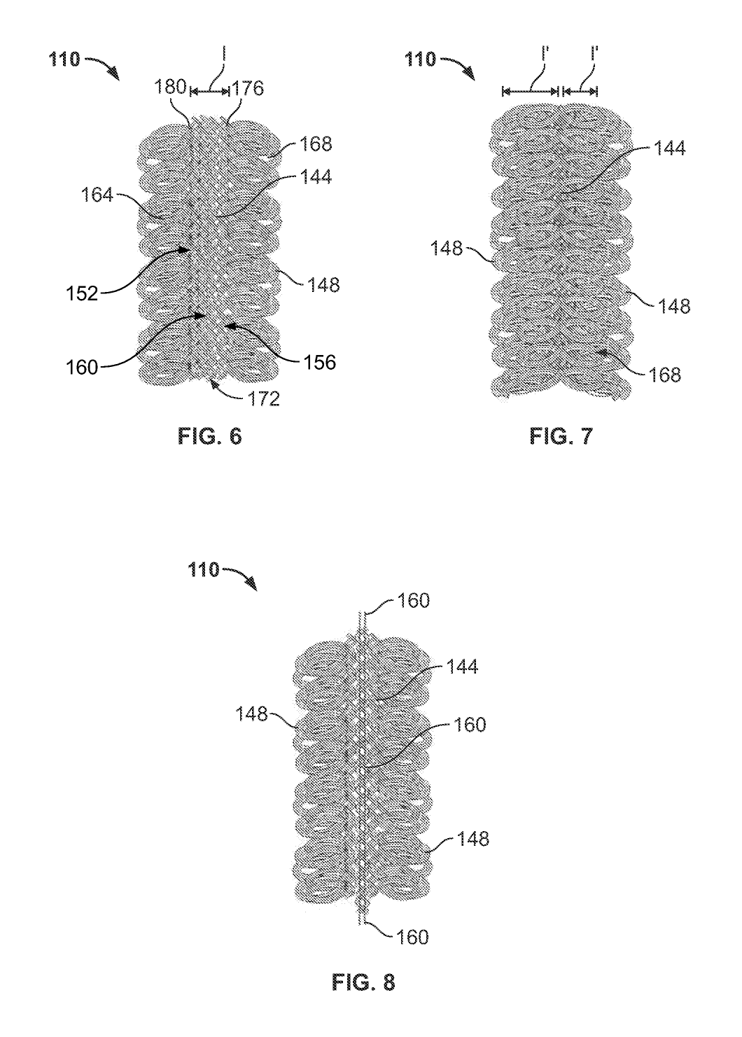

Referring now to FIGS. 6-8, an exemplary media 110 is illustrated. The illustrated media 110 is one of a variety of different types of media 110 that can be utilized in the container 32 and is not meant to be limiting. The illustrated media 110 is a looped cord media, which comprises an elongated member 144 and a plurality of loops positioned along the elongated member 144. In the illustrated exemplary embodiment, the elongated member 144 is an elongated central core of the media 110. As used herein, elongated refers to the longer of two dimensions of the media 110. In the illustrated exemplary embodiment, the vertical dimension of the media 110 is the elongated dimension. In other exemplary embodiments, the horizontal dimension or other dimension may be the elongated dimension.

Referring now to FIG. 6, an exemplary embodiment of the looped cord media 110 is illustrated. The media 110 of FIG. 6 comprises an elongated central core 144 including a first side 152 and a second side 156, a plurality of projections or media members 148 (loops in the illustrated exemplary embodiment) extending laterally from each of the first and second sides 152 and 156 and a reinforcing member 160 associated with the central core 144. In this example, the reinforcing member 160 comprises the interweaving of the cord. The media 110 also includes a front portion 164 (see FIG. 6) and a back portion 168 (see FIG. 7).

The central core 144 may be constructed in various ways and of various materials. In one embodiment, the central core 144 is knitted. The central core 144 may be knitted in a variety of manners and by a variety of machines. In some embodiments, the central core 144 can be knitted by knitting machines available from Comez SpA of Italy. The knitted portion of the core 144 may comprise a few (e.g., four to six), lengthwise rows of stitches 172. The interwoven knitted core 144 itself can act as the reinforcing member 160. The core 144 may be formed from yarn-like materials. Suitable yarn-like material may include, for example, polyester, polyamide, polyvinylidene chloride, polypropylene and other materials known to those of skill in the art. The yarn-like material may be of continuous filament construction, or a spun staple yarn. The lateral width/of the central core 144 is relatively narrow and is subject to variation. In some embodiments, the lateral width/is no greater than about 10.0 mm, is typically between about 3.0 mm and about 8.0 mm or between about 4.0 mm and about 6.0 mm.

As shown in FIG. 6, the plurality of loops 148 extend laterally from the first and second sides 152 and 156 of the central core 144. As can be seen, the plurality of loops 148 and the central core 144 are designed to provide a location where the algae may collect or be restrained while they are cultivating. The plurality of loops 148 offer flexibility in shape to accommodate growing colonies of algae. At the same time, the plurality of loops 148 inhibit the ascension of gas, particularly carbon dioxide, through the water, thereby increasing the amount of time the carbon dioxide resides near the algae growing on the media 110 (described in greater detail below).

The plurality of loops 148 are typically constructed of the same material as the central core 144, and may also include variable lateral widths/In this example, the lateral width l′ of each of the plurality of loops 148 may be within the range of between about 10.0 mm and about 15.0 mm and the central core 144 occupies, in this example, between about 1/7 and ⅕ of the overall lateral width of the media 110. The media 110 comprises a high filament count yarn that provides physical capture and entrainment of the water born microorganisms, such as microalgae, therein. The loop shape of the media 110 also assists with capturing the algae in a manner similar to a net.

With reference to FIGS. 6-8, the media 110 may optionally be strengthened through use of a variety of different reinforcing members. The reinforcing members may be either part of the media 110, such as interwoven threads of the media 110, or an additional reinforcing member separate from the media 110. With particular reference to FIG. 6, the media 110 may include two reinforcing members 176 and 180, with one member disposed on each side of the core 144. In such embodiments, the two reinforcing members 176 and 180 are in the form of outside wales that are part of the interwoven threads of the media 110. With particular reference to FIG. 8, the media 110 includes an additional reinforcing member 160 separate from the interwoven knitted central core 144. The additional reinforcing member extends along and interconnects with the central core 144. The material of the reinforcing member 160 typically has a higher tensile strength than that of the central core 144 and may have a range of break strengths between about 50.0 pounds and about 500 pounds. Thus, the reinforcing member 160 may be constructed of various materials, including high strength synthetic filament, tape, and stainless steel wire or other wire. Two particularly useful materials are Kevlar® and Tensylon®. In some embodiments, a plurality of additional reinforcing members 160 can be used to reinforce the media 110.

One or more reinforcing members 160 may be added to the central core 144 in various manners. A first manner in which the media 110 may be strengthened is by adding one or more reinforcing members 160 to the weft of the core 144 during the knitting step. These reinforcing members 160 may be disposed in a substantially parallel relationship to the warp of the core 144 and stitched into the composite structure of the core 144. As will be appreciated, the use of these reinforcing members allows the width of the central core 144 to be reduced relative to central cores of known media, without significantly jeopardizing the tensile strength of the core.

Another manner in which the media 110 may be strengthened includes the introduction of the one or more reinforcing members 160 in a twisting operation subsequent to the knitting step. This method allows the parallel introduction of the tensioned reinforcing members into the central core 144, with the central core 144 wrapping around these reinforcing members 160.

In addition, various manners of incorporating reinforcing members 160 may be combined. Thus, one or more reinforcing members 160 may be laid into the central core 144 during the knitting process, and then one or more reinforcing members 160 may be introduced during the subsequent twisting step. These reinforcing members 160 could be the same or different (e.g., during knitting, Kevlar® could be used, and during twisting, stainless steel wire could be introduced).

Further, the presence of the reinforcing members 160 can help provide a reduction of stretch in the media 110. Along these lines, the media 110 can hold more pounds of weight per foot of media than known structures. The media 110 can provide up to about 500 pounds of weight per foot. This has the advantages of reducing the risk of the media yielding or even breaking during use, and enables the algae cultivation system 20 to produce a larger volume of algae before requiring the algae to be removed from the media 110.

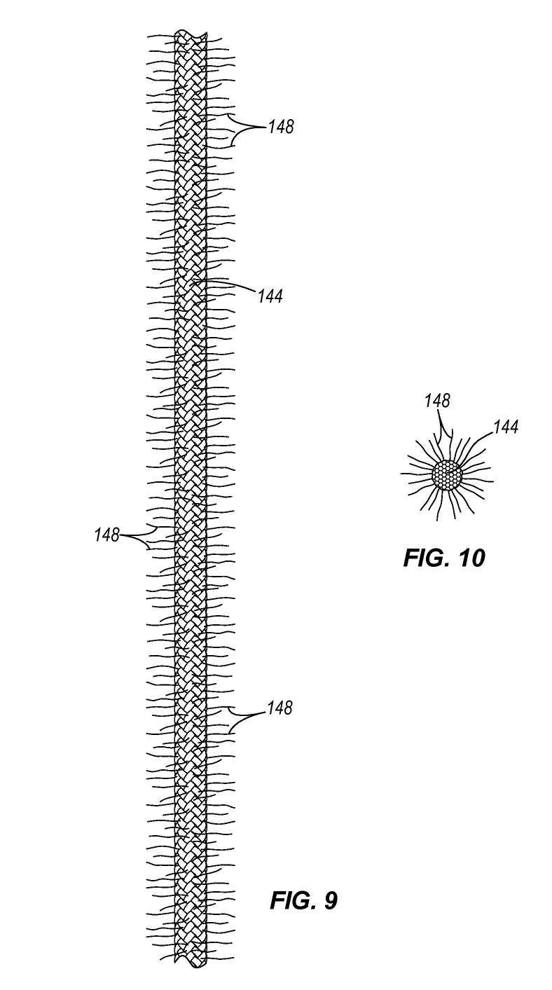

As indicated above, the illustrated exemplary media is only one of a variety of different medias that may be utilized with the system 20. Referring now to FIGS. 9 and 10, another exemplary media 110 is illustrated and includes an elongated member 144 and a plurality of projections or media members 148 projecting from the elongated member 144. In this illustrated exemplary embodiment, the elongated member 144 is an elongated central core 144, which may be a woven material, and the media members 148 may be impaled into the central core 144 such that the media members 148 are oriented substantially perpendicular to the central core 144. The media members 148 are not loops, but instead are substantially linear strands of material projecting outward away from the central core 144. When used in a container 32, the central core 144 extends vertically between the upper and lower connector plates 112, 116 and the media members 148 are oriented substantially horizontal. Algae present in the container 32 may rest or adhere to the central core 144 and the media members 148, thereby providing similar benefits to that of the exemplary media 110 described above and illustrated in FIGS. 6-8.

With continued reference to FIGS. 9 and 10, the central core 144 may be comprised of a variety of materials and be formed by a variety of manners. For example, the central core 144 may be comprised of a knitted fiber construction made of high tensile strength synthetic material such as NYLON®, KEVLAR®, DACRON®, SPECTRA®, and other multifilament twisted fibers such as polyester and polyvinylidene. The construction may be re-enforced with metal threads and monofilaments that exhibit light guiding properties. Also, for example, the central core 144 may be formed by one or more of the following manners: Knitted, extruded, molded, teased, bonded, etc. Regarding the media members 148, the media members 148 may be comprised of a variety of materials and may be introduced into or formed with the central core 144 in a variety of manners. For example, the media members 148 may be comprised of one or more of the following materials: NYLON®, KEVLAR®, DACRON®, SPECTRA®, and other multifilament twisted fibers such as polyester and polyvinylidene chloride. It should be understood that the media members 148 may be comprised of the same material as the central core 144 or may be comprised of a different material than the central core 144. Also, for example, the media members 148 may be introduced into or formed with the central core 144 in one of the following manners: Knitted, tufted, injected, extruded, molded, teased, etc.

The exemplary media 110 described herein and illustrated in FIGS. 9 and 10 may have similar characteristics and features as the exemplary media 110 described above and illustrated in FIGS. 6-8. For example, the media 110 illustrated in FIGS. 9 and 10 may have any of the forms of reinforcing members described above in connection with the media 110 illustrated in FIGS. 6-8.

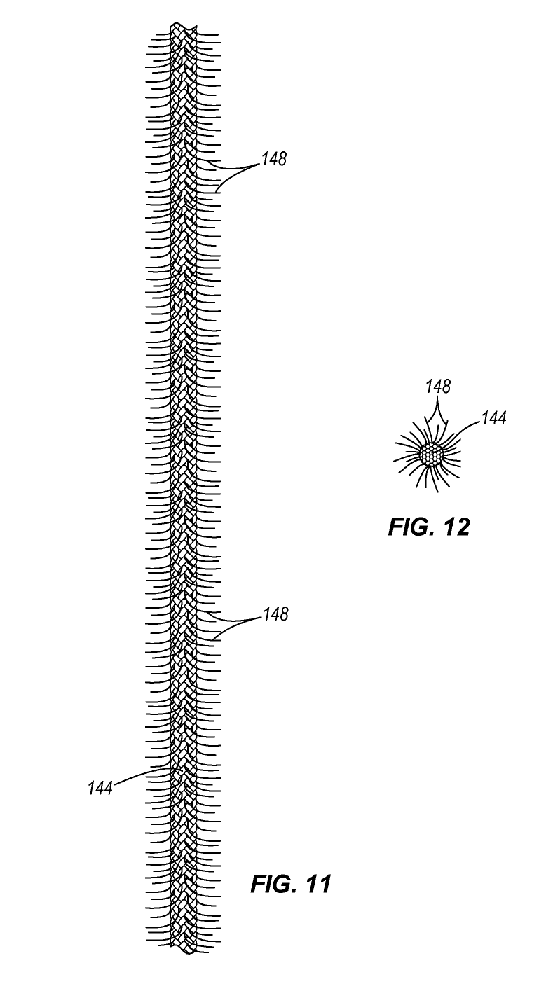

Referring now to FIGS. 11 and 12, another exemplary media is illustrated and includes an elongated member 144 and a plurality of projections or media members 148 projecting from the elongated member 144. In this illustrated exemplary embodiment, the elongated member 144 is an elongated central core 144, which may be a woven material, and the media members 148 may be woven into the central core 144 such that the media members 148 are oriented substantially perpendicular to the central core 144. The media members 148 are not loops, but instead are substantially linear strands of material projecting outward away from the central core 144. When used in a container 32, the central core 144 extends vertically between the upper and lower connector plates 112, 116 and the media members 148 are oriented substantially horizontal. Algae present in the container 32 may rest or adhere to the central core 144 and the media members 148, thereby providing similar benefits to that of the exemplary medias 110 described above and illustrated in FIGS. 6-10.

With continued reference to FIGS. 11 and 12, the central core 144 may be comprised of a variety of materials and be formed by a variety of manners. For example, the central core 144 may be comprised of a knitted fiber construction made of high tensile strength synthetic material such as NYLON®, KEVLAR®, DACRON®, SPECTRA®, and other multifilament twisted fibers such as polyester and polyvinylidene chloride. The construction may be re-enforced with metal threads and monofilaments that exhibit light guiding properties. Also, for example, the central core 144 may be formed by one or more of the following manners: Knitted, tufted, injected, molded, teased, extruded, bonded, etc. Regarding the media members 148, the media members 148 may be comprised of a variety of materials and may be introduced into or formed with the central core 144 in a variety of manners. For example, the media members 148 may be comprised of one or more of the following materials: NYLON®, KEVLAR®, DACRON®, SPECTRA®, and other monofilament twisted fibers such as polyester and polyvinylidene chloride. Materials may also exhibit light guiding properties. It should be understood that the media members 148 may be comprised of the same material as the central core 144 or may be comprised of a different material than the central core 144. Also, for example, the media members 148 may be introduced into or formed with the central core 144 in one of the following manners: Knitted, tufted, injected, molded, teased, etc.

The exemplary media 110 described herein and illustrated in FIGS. 11 and 12 may have similar characteristics and features as the exemplary medias 110 described above and illustrated in FIGS. 6-10. For example, the media 110 illustrated in FIGS. 11 and 12 may have any of the forms of reinforcing members described above in connection with the media 110 illustrated in FIGS. 6-8.

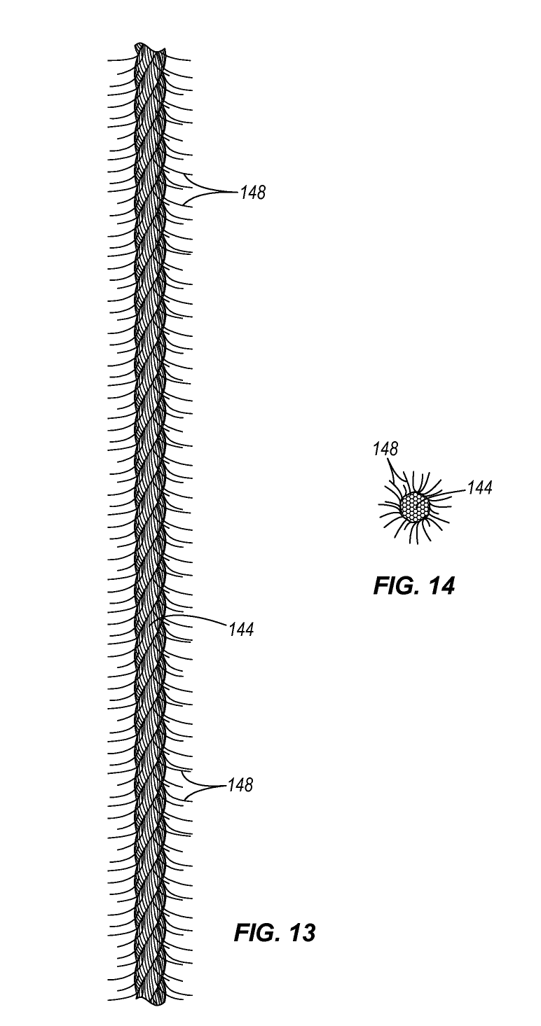

Referring now to FIGS. 13 and 14, another exemplary media is illustrated and includes an elongated member 144 and a plurality of projections or media members 148 projecting from the elongated member 144. In this illustrated exemplary embodiment, the elongated member 144 is an elongated central core 144, which may be a yarn material or other material that may fray, and the media members 148 may be formed by teasing or otherwise disturbing the yarn material. When used in a container 32, the central core 144 extends vertically between the upper and lower connector plates 112, 116 and the media members 148 project outwardly from the central core 144. Algae present in the container 32 may rest or adhere to the central core 144 and the media members 148, thereby providing similar benefits to that of the exemplary medias 110 described above and illustrated in FIGS. 6-12.

With continued reference to FIGS. 13 and 14, the central core 144 may be comprised of a variety of materials and be formed by a variety of manners. For example, the central core 144 may be formed by one or more of the following manners: Knitted, tufted, injected, extruded, molded, teased, bonded, etc. Since the media members 148 are formed by teasing or otherwise disturbing the central core 144, the media members 148 are comprised of the same material as the central core 144.

The exemplary media 110 described herein and illustrated in FIGS. 13 and 14 may have similar characteristics and features as the exemplary medias 110 described above and illustrated in FIGS. 6-12. For example, the media 110 illustrated in FIGS. 13 and 14 may have any of the forms of reinforcing members described above in connection with the media 110 illustrated in FIGS. 6-8.

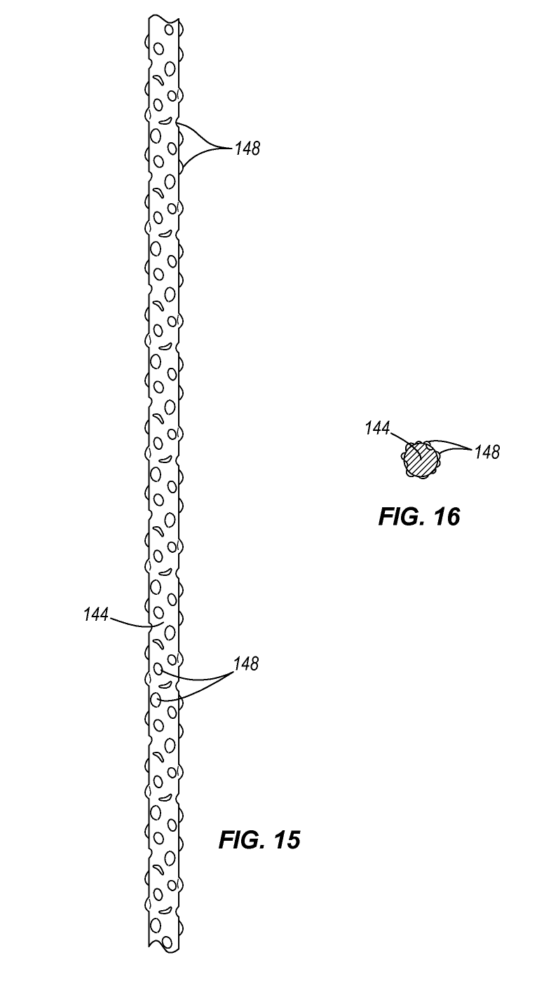

Referring now to FIGS. 15 and 16, another exemplary media is illustrated and includes an elongated member 144 and a plurality of projections or media members 148 projecting from the elongated member 144. In this illustrated exemplary embodiment, the elongated member 144 is an elongated central core 144, which may be comprised of a solid material that is scratched, chipped, scoured, roughed, dented, stippled, gouged, or otherwise imperfected to provide the media members 148 that project from the central core 144. When used in a container 32, the central core 144 extends vertically between the upper and lower connector plates 112, 116 and the media members 148 project from the central core 144 in a substantially horizontal manner. Algae present in the container 32 may rest or adhere to the central core 144 and the media members 148, thereby providing similar benefits to that of the exemplary medias 110 described above and illustrated in FIGS. 6-14.

With continued reference to FIGS. 15 and 16, the central core 144 may be comprised of a variety of materials and be formed by a variety of manners. For example, the central core 144 may be comprised of a knitted fiber construction made of high tensile strength synthetic material such as NYLON®, KEVLAR®, DACRON®, SPECTRA®, and other multifilament twisted fibers such as polyester and polyvinylidene chloride. The construction may be re-enforced with metal threads and monofilaments that exhibit light guiding properties. Also, for example, the central core 144 may be formed by one or more of the following manners: Knitted, tufted, injected, molded, teased, bonded, etc. Since the media members 148 are formed by imperfecting the outer surface of the central core 144, the media members 148 are comprised of the same material as the central core 144.

The exemplary media 110 described herein and illustrated in FIGS. 15 and 16 may have similar characteristics and features as the exemplary medias 110 described above and illustrated in FIGS. 6-14. For example, the media 110 illustrated in FIGS. 15 and 16 may have any of the forms of reinforcing members described above in connection with the media 110 illustrated in FIGS. 6-8.

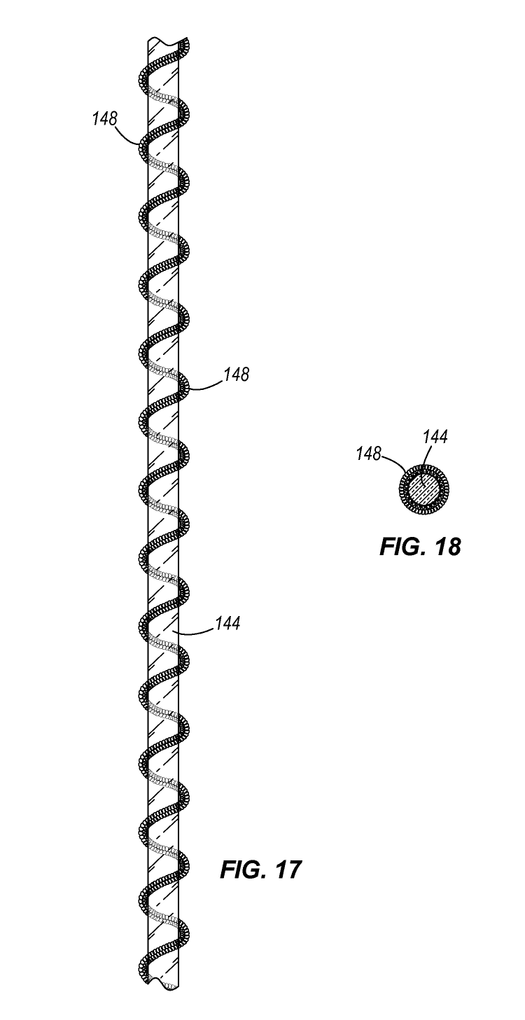

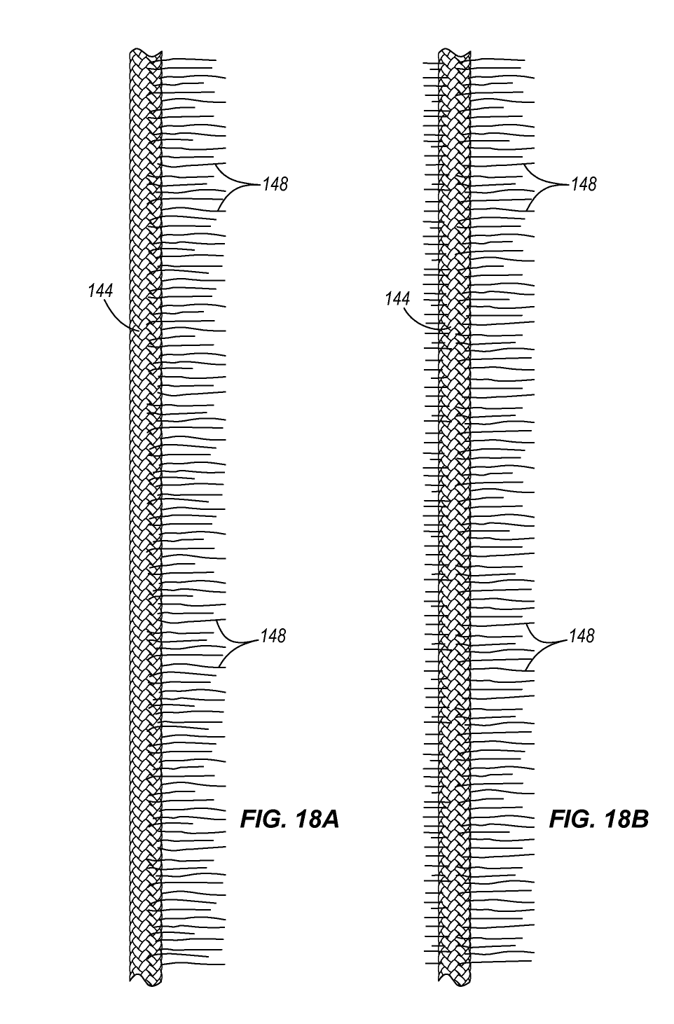

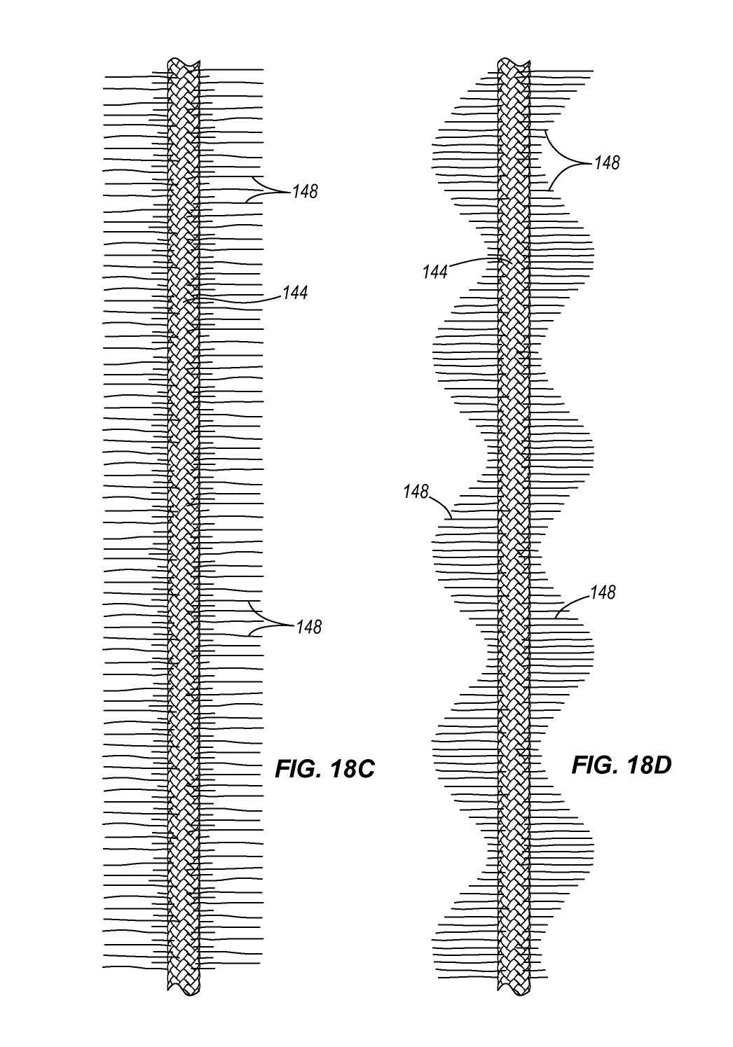

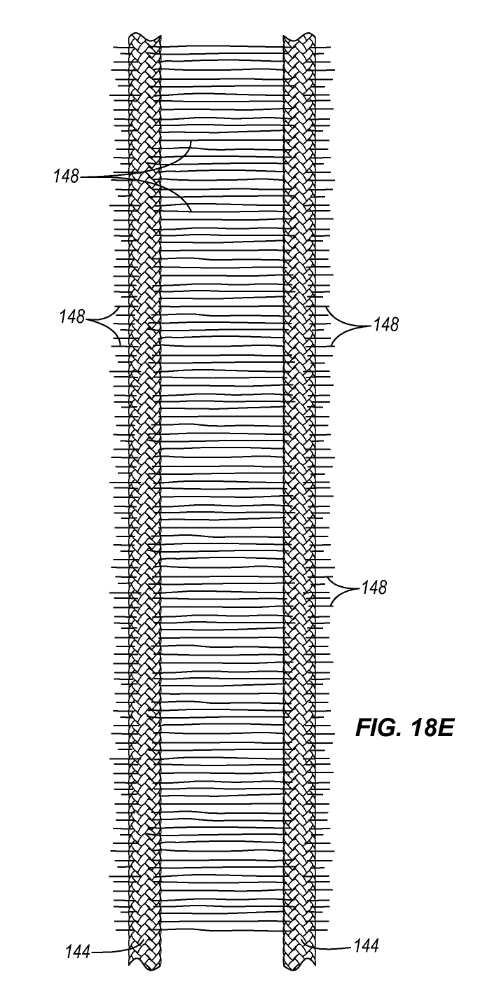

Referring now to FIGS. 17 and 18, another exemplary media is illustrated and includes an elongated member 144 and a plurality of projections or media members 148 projecting from the elongated member 144. In this illustrated exemplary embodiment, the elongated member 144 is an elongated central core 144, which may be comprised of a material that easily transmits and emits light therefrom, and the media members 148 comprise one or more media strands wound closely around the central core 144. One or more light sources may emit light into the central core 144 of this exemplary media 110 and the media 110 then will emit the light therefrom. Algae present in the container 32 may rest or adhere to the central core 144 and the media members 148. Due to the close winding of the media members 148 and the central core 144, the light emitted from the central core 144 will emit onto the media members 148 and the media thereon. In some embodiments of this exemplary media 110, the outer surface of the central core 144 may be, for example, scratched, chipped, scoured, roughed, dented, stippled, gouged, or otherwise imperfected, to assist with diffraction of the light from the interior to the exterior of the central core 144.

With continued reference to FIGS. 17 and 18, the central core 144 may be comprised of a variety of materials and be formed by a variety of manners. For example, the central core 144 may be comprised of one or more of the following materials: NYLON®, KEVLAR®, DACRON®, SPECTRA®, and other monofilament and multifilament twisted fibers such as polyester and polyvinylidene chloride. Materials may also exhibit light guiding properties. Also, for example, the central core 144 may be formed by one or more of the following manners: Knitted, tufted, injected, extruded, molded, teased, bonded, etc. Regarding the media members 148, the media members 148 may be comprised of a variety of materials and may have a variety of configurations. For example, the media members 148 may be comprised of one or more of the following materials: NYLON®, KEVLAR®, DACRON®, SPECTRA®, and other monofilament and multifilament twisted fibers such as polyester and polyvinylidene chloride. Materials may also exhibit light guiding properties. Also, for example, the media members 148 wound around the central core 144 may have a variety of different configurations such as loop cord media similar to that illustrated in FIGS. 6-8, any of the other exemplary media illustrated in FIGS. 9-16, or other shapes, sizes, and configurations.

The exemplary media 110 described herein and illustrated in FIGS. 17 and 18 may have similar characteristics and features as the exemplary medias 110 described above and illustrated in FIGS. 6-16. For example, the media 110 illustrated in FIGS. 17 and 18 may have any of the forms of reinforcing members described above in connection with the media 110 illustrated in FIGS. 6-8.

Referring now to FIG. 18A, another exemplary media is illustrated and includes an elongated member 144 and a plurality of projections or media members 148 projecting from the elongated member 144. In this illustrated exemplary embodiment, the elongated member 144 is disposed at an end of the media members 148 and the media members 148 extend to one side of the elongated member 144. In some exemplary embodiments, the elongated member 144 may be a woven material and the media members 148 may be woven into the elongated member 144 such that the media members 148 are oriented substantially perpendicular to the elongated member 144. In the illustrated exemplary embodiment, the media members 148 are substantially linear strands of material projecting outward away from the elongated member 144. In other exemplary embodiments, the media members 148 may be loops. When used in a container 32, the elongated member 144 extends vertically between the upper and lower connector plates 112, 116 and the media members 148 are oriented substantially horizontal. Algae present in the container 32 may rest or adhere to the elongated member 144 and the media members 148, thereby providing similar benefits to that of the exemplary medias 110 described above and illustrated in FIGS. 6-18.

With continued reference to FIG. 18A, the elongated member 144 may be comprised of a variety of materials and be formed by a variety of manners. For example, the elongated member 144 may be comprised of a knitted fiber construction made of high tensile strength synthetic material such as NYLON®, KEVLAR®, DACRON®, SPECTRA®, and other multifilament twisted fibers such as polyester and polyvinylidene chloride. The construction may be re-enforced with metal threads and monofilaments that exhibit light guiding properties. Also, for example, the elongated member 144 may be formed by one or more of the following manners: Knitted, tufted, injected, molded, teased, extruded, bonded, etc. Regarding the media members 148, the media members 148 may be comprised of a variety of materials and may be introduced into or formed with the elongated member 144 in a variety of manners. For example, the media members 148 may be comprised of one or more of the following materials: NYLON®, KEVLAR®, DACRON®, SPECTRA®, and other monofilament twisted fibers such as polyester and polyvinylidene chloride. Materials may also exhibit light guiding properties. It should be understood that the media members 148 may be comprised of the same material as the elongated member 144 or may be comprised of a different material than the elongated member 144. Also, for example, the media members 148 may be introduced into or formed with the elongated member 144 in one of the following manners: Knitted, tufted, injected, molded, teased, etc.

The exemplary media 110 described herein and illustrated in FIG. 18A may have similar characteristics and features as the exemplary medias 110 described above and illustrated in FIGS. 6-18. For example, the media 110 illustrated in FIG. 18A may have any of the forms of reinforcing members described above in connection with the media 110 illustrated in FIGS. 6-8.

Referring now to FIG. 18B, another exemplary media is illustrated and includes an elongated member 144 and a plurality of projections or media members 148 projecting from the elongated member 144. In this illustrated exemplary embodiment, the elongated member 144 is disposed near an end of and displaced from a center of the media members 148. In some exemplary embodiments, the elongated member 144 may be a woven material and the media members 148 may be woven into the elongated member 144 such that the media members 148 are oriented substantially perpendicular to the elongated member 144. In the illustrated exemplary embodiment, the media members 148 are substantially linear strands of material projecting outward away from the elongated member 144. In other exemplary embodiments, the media members 148 may be loops. When used in a container 32, the elongated member 144 extends vertically between the upper and lower connector plates 112, 116 and the media members 148 are oriented substantially horizontal. Algae present in the container 32 may rest or adhere to the elongated member 144 and the media members 148, thereby providing similar benefits to that of the exemplary medias 110 described above and illustrated in FIGS. 6-18A.

With continued reference to FIG. 18B, the elongated member 144 may be comprised of a variety of materials and be formed by a variety of manners. For example, the elongated member 144 may be comprised of a knitted fiber construction made of high tensile strength synthetic material such as NYLON®, KEVLAR®, DACRON®, SPECTRA®, and other multifilament twisted fibers such as polyester and polyvinylidene chloride. The construction may be re-enforced with metal threads and monofilaments that exhibit light guiding properties. Also, for example, the elongated member 144 may be formed by one or more of the following manners: Knitted, tufted, injected, molded, teased, extruded, bonded, etc. Regarding the media members 148, the media members 148 may be comprised of a variety of materials and may be introduced into or formed with the elongated member 144 in a variety of manners. For example, the media members 148 may be comprised of one or more of the following materials: NYLON®, KEVLAR®, DACRON®, SPECTRA®, and other monofilament twisted fibers such as polyester and polyvinylidene chloride. Materials may also exhibit light guiding properties. It should be understood that the media members 148 may be comprised of the same material as the elongated member 144 or may be comprised of a different material than the elongated member 144. Also, for example, the media members 148 may be introduced into or formed with the elongated member 144 in one of the following manners: Knitted, tufted, injected, molded, teased, etc.

The exemplary media 110 described herein and illustrated in FIG. 18B may have similar characteristics and features as the exemplary medias 110 described above and illustrated in FIGS. 6-18A. For example, the media 110 illustrated in FIG. 18B may have any of the forms of reinforcing members described above in connection with the media 110 illustrated in FIGS. 6-8.

Referring now to FIG. 18C, another exemplary media is illustrated and includes an elongated member 144 and a plurality of projections or media members 148 projecting from the elongated member 144. In this illustrated exemplary embodiment, the elongated member 144 is disposed near an end of and displaced from a center of the media members 148. In some exemplary embodiments, the elongated member 144 may be a woven material and the media members 148 may be woven into the elongated member 144 such that the media members 148 are oriented substantially perpendicular to the elongated member 144. In the illustrated exemplary embodiment, the media members 148 are substantially linear strands of material projecting outward away from the elongated member 144. In other exemplary embodiments, the media members 148 may be loops. When used in a container 32, the elongated member 144 extends vertically between the upper and lower connector plates 112, 116 and the media members 148 are oriented substantially horizontal. Algae present in the container 32 may rest or adhere to the elongated member 144 and the media members 148, thereby providing similar benefits to that of the exemplary medias 110 described above and illustrated in FIGS. 6-18B.

With continued reference to FIG. 18C, the elongated member 144 may be comprised of a variety of materials and be formed by a variety of manners. For example, the elongated member 144 may be comprised of a knitted fiber construction made of high tensile strength synthetic material such as NYLON®, KEVLAR®, DACRON®, SPECTRA®, and other multifilament twisted fibers such as polyester and polyvinylidene chloride. The construction may be re-enforced with metal threads and monofilaments that exhibit light guiding properties. Also, for example, the elongated member 144 may be formed by one or more of the following manners: Knitted, tufted, injected, molded, teased, extruded, bonded, etc. Regarding the media members 148, the media members 148 may be comprised of a variety of materials and may be introduced into or formed with the elongated member 144 in a variety of manners. For example, the media members 148 may be comprised of one or more of the following materials: NYLON®, KEVLAR®, DACRON®, SPECTRA®, and other monofilament twisted fibers such as polyester and polyvinylidene chloride. Materials may also exhibit light guiding properties. It should be understood that the media members 148 may be comprised of the same material as the elongated member 144 or may be comprised of a different material than the elongated member 144. Also, for example, the media members 148 may be introduced into or formed with the elongated member 144 in one of the following manners: Knitted, tufted, injected, molded, teased, etc.

The exemplary media 110 described herein and illustrated in FIG. 18C may have similar characteristics and features as the exemplary medias 110 described above and illustrated in FIGS. 6-18B. For example, the media 110 illustrated in FIG. 18C may have any of the forms of reinforcing members described above in connection with the media 110 illustrated in FIGS. 6-8.

Referring now to FIG. 18D, another exemplary media is illustrated and includes an elongated member 144 and a plurality of projections or media members 148 projecting from the elongated member 144. In this illustrated exemplary embodiment, the elongated member 144 is disposed at different locations along the various media members 148. In some exemplary embodiments, the elongated member 144 may be a woven material and the media members 148 may be woven into the elongated member 144 such that the media members 148 are oriented substantially perpendicular to the elongated member 144. In the illustrated exemplary embodiment, the media members 148 are substantially linear strands of material projecting outward away from the elongated member 144. In other exemplary embodiments, the media members 148 may be loops. When used in a container 32, the elongated member 144 extends vertically between the upper and lower connector plates 112, 116 and the media members 148 are oriented substantially horizontal. Algae present in the container 32 may rest or adhere to the elongated member 144 and the media members 148, thereby providing similar benefits to that of the exemplary medias 110 described above and illustrated in FIGS. 6-18C.

With continued reference to FIG. 18D, the elongated member 144 may be comprised of a variety of materials and be formed by a variety of manners. For example, the elongated member 144 may be comprised of a knitted fiber construction made of high tensile strength synthetic material such as NYLON®, KEVLAR®, DACRON®, SPECTRA®, and other multifilament twisted fibers such as polyester and polyvinylidene chloride. The construction may be re-enforced with metal threads and monofilaments that exhibit light guiding properties. Also, for example, the elongated member 144 may be formed by one or more of the following manners: Knitted, tufted, injected, molded, teased, extruded, bonded, etc. Regarding the media members 148, the media members 148 may be comprised of a variety of materials and may be introduced into or formed with the elongated member 144 in a variety of manners. For example, the media members 148 may be comprised of one or more of the following materials: NYLON®, KEVLAR®, DACRON®, SPECTRA®, and other monofilament twisted fibers such as polyester and polyvinylidene chloride. Materials may also exhibit light guiding properties. It should be understood that the media members 148 may be comprised of the same material as the elongated member 144 or may be comprised of a different material than the elongated member 144. Also, for example, the media members 148 may be introduced into or formed with the elongated member 144 in one of the following manners: Knitted, tufted, injected, molded, teased, etc.

The exemplary media 110 described herein and illustrated in FIG. 18D may have similar characteristics and features as the exemplary medias 110 described above and illustrated in FIGS. 6-18C. For example, the media 110 illustrated in FIG. 18D may have any of the forms of reinforcing members described above in connection with the media 110 illustrated in FIGS. 6-8.

Referring now to FIG. 18E, another exemplary media is illustrated and includes a pair of elongated members 144 and a plurality of projections or media members 148 projecting from and extending between the elongated members 144. In this illustrated exemplary embodiment, the elongated members 144 are disposed near ends of and displaced from centers of the media members 148. In some exemplary embodiments, the elongated members 144 may be a woven material and the media members 148 may be woven into the elongated members 144 such that the media members 148 are oriented substantially perpendicular to the elongated members 144. In the illustrated exemplary embodiment, the media members 148 are substantially linear strands of material projecting outward away from the elongated members 144. In other exemplary embodiments, the media members 148 may be loops. When used in a container 32, the elongated members 144 extends vertically between the upper and lower connector plates 112, 116 and the media members 148 are oriented substantially horizontal. Algae present in the container 32 may rest or adhere to the elongated members 144 and the media members 148, thereby providing similar benefits to that of the exemplary medias 110 described above and illustrated in FIGS. 6-18D.

With continued reference to FIG. 18E, the elongated members 144 may be comprised of a variety of materials and be formed by a variety of manners. For example, the elongated members 144 may be comprised of a knitted fiber construction made of high tensile strength synthetic material such as NYLON®, KEVLAR®, DACRON®, SPECTRA®, and other multifilament twisted fibers such as polyester and polyvinylidene chloride. The construction may be re-enforced with metal threads and monofilaments that exhibit light guiding properties. Also, for example, the elongated members 144 may be formed by one or more of the following manners: Knitted, tufted, injected, molded, teased, extruded, bonded, etc. Regarding the media members 148, the media members 148 may be comprised of a variety of materials and may be introduced into or formed with the elongated members 144 in a variety of manners. For example, the media members 148 may be comprised of one or more of the following materials: NYLON®, KEVLAR®, DACRON®, SPECTRA®, and other monofilament twisted fibers such as polyester and polyvinylidene chloride. Materials may also exhibit light guiding properties. It should be understood that the media members 148 may be comprised of the same material as the elongated members 144 or may be comprised of a different material than the elongated members 144. Also, for example, the media members 148 may be introduced into or formed with the elongated members 144 in one of the following manners: Knitted, tufted, injected, molded, teased, etc.

The exemplary media 110 described herein and illustrated in FIG. 18E may have similar characteristics and features as the exemplary medias 110 described above and illustrated in FIGS. 6-18D. For example, the media 110 illustrated in FIG. 18E may have any of the forms of reinforcing members described above in connection with the media 110 illustrated in FIGS. 6-8.

The illustrated and described exemplary medias are presented as some of the many different types of media capable of being employed by the system 20 and are not intended to be limiting. Accordingly, other types of media are within the intended spirit and scope of the present invention.

With reference to FIGS. 3-5 and 19-21, connection of the media 110 to the frame 108 will be described. The media 110 can be connected to the frame 108 in a variety of manners, however, only some of the manners will be described herein. The described manners for connecting the media 110 to the frame 108 are not meant to be limiting and, as stated above, the media 110 can be connected to the frame 108 in a wide variety of manners.

The media 110 may be attached to the frame 108 of the container in a variety of manners and the manners described herein are only a few of the many manners possible. In a first exemplary manner of connection, the media 110 can be comprised of a single long strand strung back and forth between the upper and lower connector plates 112, 116. In this manner, a first end of the media strand 110 is tied or otherwise secured to either the upper connector plate 112 or the lower connector plate 116, the strand of media 110 is extended back and forth between the upper and lower connector plates 112, 116, and the second end is tied to either the upper connector plate 112 or the lower connector plate 116 depending on the length of the media strand 110 and which of the connector plates 112, 116 is nearest the second end when the media strand is fully strung. Stringing a single piece of media 110 back and forth in this manner provides a plurality of media segments 110 extending between the upper and lower connector plates 112, 116 that are spaced apart from one another. The single strand of media 110 can be strung back and forth between the upper and lower connector plates 112, 116 in a variety of manners and, for the sake of brevity, only one exemplary manner will be described herein, however, the described manner is not intended to be limiting.

The first end of the strand is tied to the upper connector plate 112 in a first one of the apertures 128 defined therein. The media strand 110 is then extended downward to the lower connector plate 116 and inserted through a first one of the apertures 128 defined in the lower connector plate 116. The media strand 110 is then inserted upward through a second one of the apertures 128 positioned adjacent to the first one of the apertures 128 defined in the lower bracket plate 116 and extended upward toward the upper connector plate 112. The media strand 110 is then inserted upwardly through a second one of the apertures 128 positioned adjacent to the first one of the apertures 128 defined in the upper connector plate 112 and then downwardly inserted through a third one of the apertures 128 positioned adjacent the second one of the apertures 128 defined in the upper connector plate 112. Extension of the media strand 110 back and forth between adjacent apertures 128 defined in the upper and lower connector plates 112, 116 continues until the media 110 has been inserted through all of the apertures 128 defined in the upper and lower connector plates 112, 116. Since the illustrated exemplary connector plates 112, 116 includes six apertures 128 and the first end of the media strand 110 is tied to one of the apertures 128 in the upper connector plate 112, the last aperture 128 to be occupied will be in the upper connector plate 112.

After the media 110 has occupied the sixth aperture 128 in the upper connector plate 112, the media strand 110 is extended into a first one of the recesses 132 in the upper connector plate 112. From this first recess 132, the media strand 110 is extended downward toward and into a first one of the recesses 132 in the lower connector plate 116. The media strand 110 then extends along a bottom surface 184 of the lower connector plate 116 and upward into a second one of the recesses 132 adjacent the first one of the recesses 132 in the lower connector plate 116. From this second recess 132, the media strand 110 extends upward and into a second one of the recesses 132 positioned adjacent the first one of the recesses 132 defined in the upper connector plate 112. The media strand 110 then extends along a top surface 188 of the upper connector plate 112 and downward into a third one of the recesses 132 adjacent the second one of the recesses 132 in the upper connector plate 112. Extension of the media strand 110 back and forth between the adjacent recesses 132 defined in the upper and lower connector plates 112, 116 continues until the media 110 has been inserted through all of the recesses 132 defined in the upper and lower connector plates 112, 116. Since the illustrated exemplary connector plates 112, 116 include ten recesses 132 and one of the recesses 132 in the upper connector plate 112 is occupied first, the last recess 132 to be occupied will be in the upper connector plate 112. After upwardly inserting the media strand 110 into the last recess 132 in the upper connector plate 112, the second end of the media strand 110 can be tied to one of the apertures 128 defined in the upper connector plate 112. To assist with securing the media strand 110 to the upper and lower connector plates 112, 116, a fastener 192 such as, for example, a wire, rope, or other thin strong and bendable device is positioned around the edge 140 of each of the upper and lower connector plates 112, 116 and tightened into a slot 136 defined in the edge 140 of each of the upper and lower connector plates 112, 116 to entrap the media strand 110 in the recesses 132 between the fasteners 192 and the upper and lower connector plates 112, 116. As indicated above, the illustrated and described manner of connecting the media strand 110 to the frame 108 is only an exemplary manner and a wide variety of alternatives exist and are within the spirit and scope of the present invention.

In the illustrated example, the apertures 128 of the upper and lower plates 112, 116 are generally vertically aligned such that an aperture 128 of the upper plate 112 aligns vertically with an aperture 128 of the lower plate 116. Similarly, the recesses 132 of the upper and lower plates 112, 116 are generally vertically aligned. As illustrated, the various extensions or segments of the media strand 110 extending between the upper and lower connector plates 112, 116 extend in a substantially vertical manner. This is achieved by extending the media strands 110 between aligned apertures 128 of the upper and lower plates 112, 116 and aligned recesses 132 of the upper and lower plates 112, 116. However, it should be understood that the media strand 110 can also extend between the upper and lower connector plates 112, 116 in an angled manner relative to the vertical such that the media strand 110 extends between unaligned apertures 128 and recesses 132.

In a second manner of connection, the media 110 can be comprised of a plurality of separate medias 110 individually strung between the upper and lower connector plates 112, 116. In this manner, each media 110 extends between the upper and lower connector plates 112, 116 a single time. A first end of the each of the medias 110 is tied or otherwise secured to one of the upper connector plate 112 or the lower connector plate 116 and the second end extends to and secures to the other of the upper connector plate 112 or the lower connector plate 116. Stringing multiple medias 110 in this manner provides a plurality of media segments 110 extending between the upper and lower connector plates 112, 116 that are spaced apart from one another. In some embodiments, the plurality of medias 110 are strung between the upper and lower connector plates 112, 116 in a substantially vertical manner, which is achieved by extending the medias 110 between aligned apertures 128 and aligned recesses 132. In other embodiments, the plurality of medias 110 are strung between the upper and lower connector plates 112, 116 in an angled manner relative to the vertical, which is achieved by extending the medias 110 between unaligned apertures 128 and unaligned recesses 132.

It should be understood that the media or medias 110 may be coupled to the upper and lower connector plates 112, 116 in a variety of manners other than those described herein. For example, the media or medias 110 may be clipped, adhered, fastened, or secured to the frame 108 in any other appropriate manner.

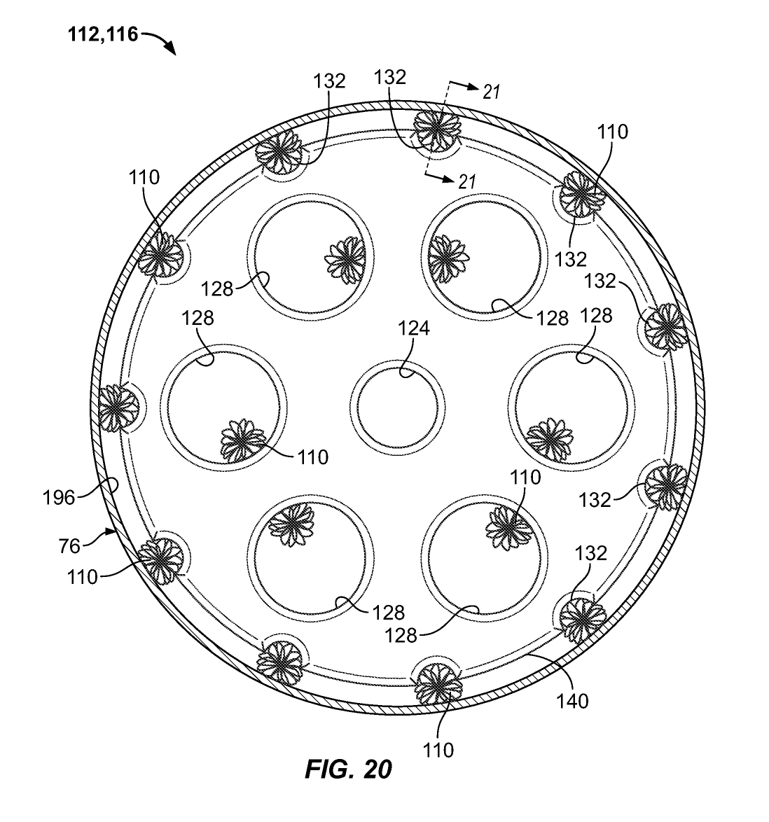

With particular reference to FIG. 20, the illustrated exemplary orientation of the media 110 provides for a more dense concentration of media 110 near the center of the container 32 (i.e., near the shaft 120) than toward the outer periphery of the container 32. This orientation of the media 110 facilitates, among other things, penetration of sunlight past the outermost strands of media 110 and into the center of the container 32 where the inner media strands 110 are located, thereby facilitating efficient photosynthesis and cultivation of the algae located on the interior media strands 110. If, on the other hand, the media 110 is more dense near the outer periphery of the container 32, the dense outer media 110 would block a significant amount of the sunlight, thereby inhibiting penetration of the sunlight to interior of the container 32 and inhibiting photosynthesis and cultivation of the algae located on the interior media strands 110. With the media 110 strung between the upper and lower connector plates 112, 116 in these described embodiments, the media 110 provides a treacherous path for gases (e.g., carbon dioxide) that are ascending through the water in the container 32. This treacherous path slows the ascension of the gas bubbles, thereby facilitating increased contact time between the gas bubbles and the algae supported on the media 110.

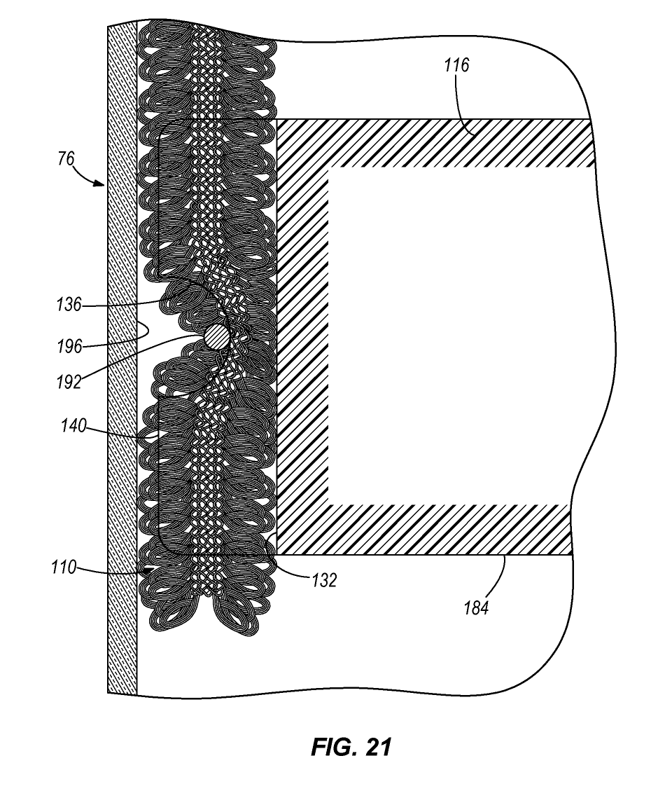

No matter the manner used to connect the media 110 to the upper and lower connector plates 112, 116, outermost strands of the media 110 extending between the recesses 132 defined in the periphery of the upper and lower connector plates 112, 116 project externally of the outer edges 140 of the upper and lower connector plates 112, 116. By extending externally of the outer edges 140 of the connector plates 112, 116, the media strands 110 engage an interior surface 196 of the housing 76 (the purpose of which will be described in greater detail below) as best illustrated in FIGS. 20 and 21.

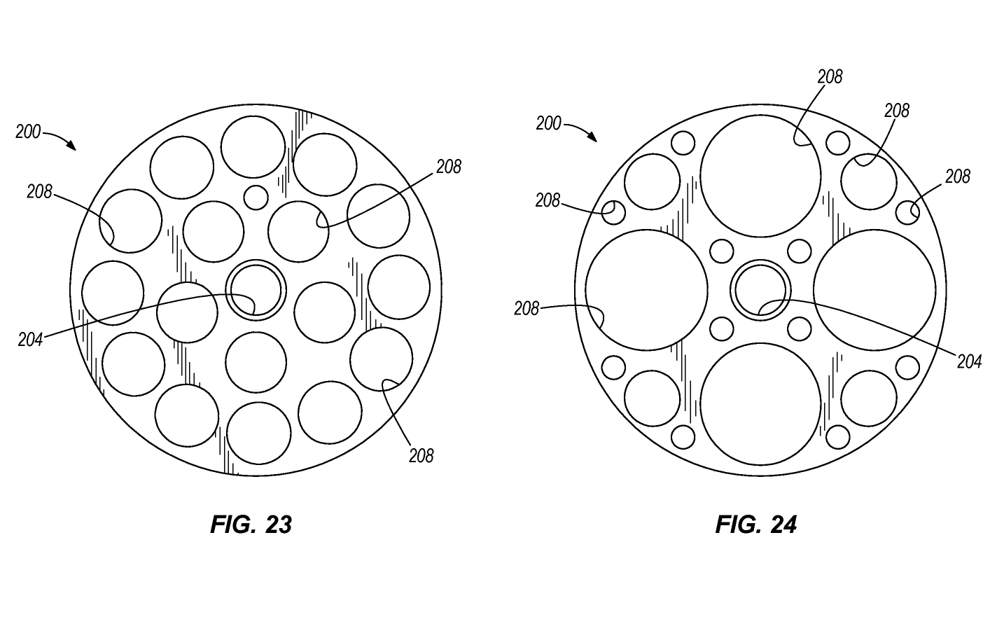

Referring now to FIGS. 3, 4, and 22, the container 32 also includes an exemplary bushing 200 positioned within the housing 76. The bushing 200 is substantially circular in shape and disposed near a bottom of the housing 76. The bushing 200 includes a central opening 204 receiving an end of the shaft 120 and provides support to the end of the shaft 120. In addition, the bushing 200 maintains proper positioning of the frame 108 relative to the housing 76. In this example, the shaft 120 is loosely confined within the central opening 204 and the bushing inhibits substantial lateral movement of the shaft 120. The bushing 200 includes a plurality of gas apertures 208 that allow gas introduced into the bottom of the container 32 to permeate through the bushing 200. The bushing 200 can include any number and any size of apertures 208 as long as the bubbles satisfactorily permeate the bushing 200. With particular reference to FIGS. 23 and 24, two additional examples of the bushing 200 are illustrated. As can be seen, the bushings 200 include different configurations and sizes of holes 208.

Referring back to FIGS. 3 and 4, the container 32 further includes a top cap or cover 212 positioned at the top of the housing 76 to close-off and seal the top of the housing 76, thereby sealing the container 32 from the external environment In some embodiments, the cover 212 is a close fitted plastic cap such as, for example, a PVC clean-out coupling that is capable of screwing into and unscrewing from the container. Alternatively, the cover 212 can be a wide variety of objects as long as the object sufficiently seals the top of the housing 76. The cover 212 also includes a central opening 216 and a bearing disposed in the central opening 216 for receiving the shaft 120 and facilitating rotation of the shaft 120 relative to the cover 212 (described in greater detail below). The shaft 120 extends below the cover 212 into the housing 76 and a portion of the shaft 120 remains above the cover 212. A drive pulley or gear 220 is connected to the portion of the shaft 120 disposed above the cover 212 and is rigidly secured to the shaft 120 to prevent relative movement of the gear 220 and the shaft 120. The gear 220 is coupled to a drive mechanism including a drive member 224 and a belt or chain 228. The drive member 224 is operable to rotate the gear 220 and shaft 120, thereby rotating the frame 108 relative to the housing 76 (described in greater detail below). In the illustrated exemplary embodiment, the drive member 224 may be an AC or DC motor. Alternatively, the drive member 224 may be a wide variety of other types of drive members such as, for example, a fuel power engine, a wind powered drive member, a pneumatic powered drive member, a human powered drive member, etc.

As indicated above, it may be desirable to provide an artificial light system 37 to supplement or substitute natural sunlight 72 for purposes of driving photosynthesis of the algae. The artificial light system 37 may take many shapes and forms, and may operate in a variety of manners. Several exemplary artificial light systems 37 are illustrated and described herein, however, these exemplary artificial light systems 37 are not intended to be limiting and other artificial light systems are contemplated and are within the spirit and scope of the present invention.

With reference to FIGS. 25 and 26, an exemplary embodiment of the artificial light system 37 is shown. This exemplary artificial light system 37 is one of many types of artificial light systems contemplated and is not intended to be limiting. The exemplary artificial light system 37 is capable of extending the period of time in which the algae is exposed to light or is capable of supplementing the natural sunlight 72 absorbed by the algae. In the illustrated example, the artificial light system 37 includes a base 39 and a light source such as an array of light emitting diodes (LEDs) 41 connected to the base 39. The base 39 and LEDs 41 are positioned on a dark side of each container 32. LEDs 41 have been shown to operate at low voltages, thereby consuming very little energy, and do not generate undesirable quantities of heat. The dark side of a container 32 is the side of the container 32 that receives the least amount of sunlight 72. For example, in a container 32 positioned in the northern hemisphere of the Earth during the winter season, the sun is low in the sky to the south, thereby emitting the most sunlight 72 toward a southern side of the container 32. In this example, the dark side is the north side of the container 32. Accordingly, the array of LEDs 41 is positioned on the north side of the container 32.

In some embodiments, the LEDs 41 may have a frequency range between about 400 nanometers (nm) to about 700 nanometers. The artificial lighting system 37 may include only single frequency LEDs 41 thereon or may include a variety of different frequency LEDs 41, thereby providing a broad spectrum of frequencies. In other embodiments, the LEDs 41 may utilize only a limited portion of the light spectrum rather than the entire light spectrum. With such limited use of the light spectrum. LEDs consume less energy. Exemplary portions of the light spectrum utilized by the LEDs may include the blue spectrum (i.e., frequencies between about 400 and about 500 nanometers) and the red spectrum (i.e., frequencies between about 600 and about 800 nanometers). LEDs may emit light from other portions of the light spectrum and at other frequencies and still be within the intended spirit and scope of the present invention.

In some exemplary embodiments, the base 39 may be reflective in nature for reflecting sunlight 72 onto the dark side of the container 32 or some other portion of the container 32. In such embodiments, sunlight 72 passing through, missing, or otherwise not being emitted into or onto the container 32 may engage the reflective base 39 and reflect onto and into the container 32.

In other embodiments, the artificial light system 37 may include light sources 41 other than LEDs such as, for example, fluorescents, light conducting fibers, etc. In yet other embodiments, the artificial light system 37 may include a plurality of fiber optic light channels arranged around the container 32 to emit light onto the container 32. In such embodiments, the fiber optic light channels may receive light in a variety of manners including LEDs or other light emitting devices or from a solar light collection apparatus oriented to receive sunlight 72 and transfer the collected sunlight 72 to the light channels via fiber optic cables.

In addition, the light emitted by the artificial light system 37 may be emitted either continuously or may be flashed at a desired rate. Flashing the LEDs 41 mimics conditions in natural water such as light diffraction by wave action and inconsistent light intensities caused by varying water clarity. In some examples, the light may be flashed at a rate of about 37 KHz, which has been shown to produce a 20% higher algae yield than when the LEDs 41 emit continuous light. In other examples, the light may be flashed between a range of about 5 KHz to about 37 KHz.

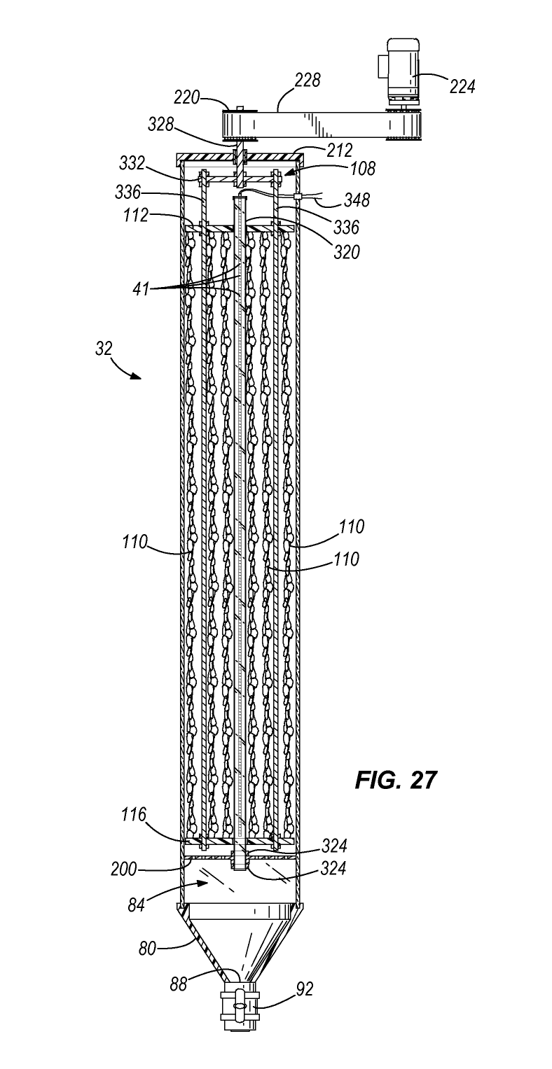

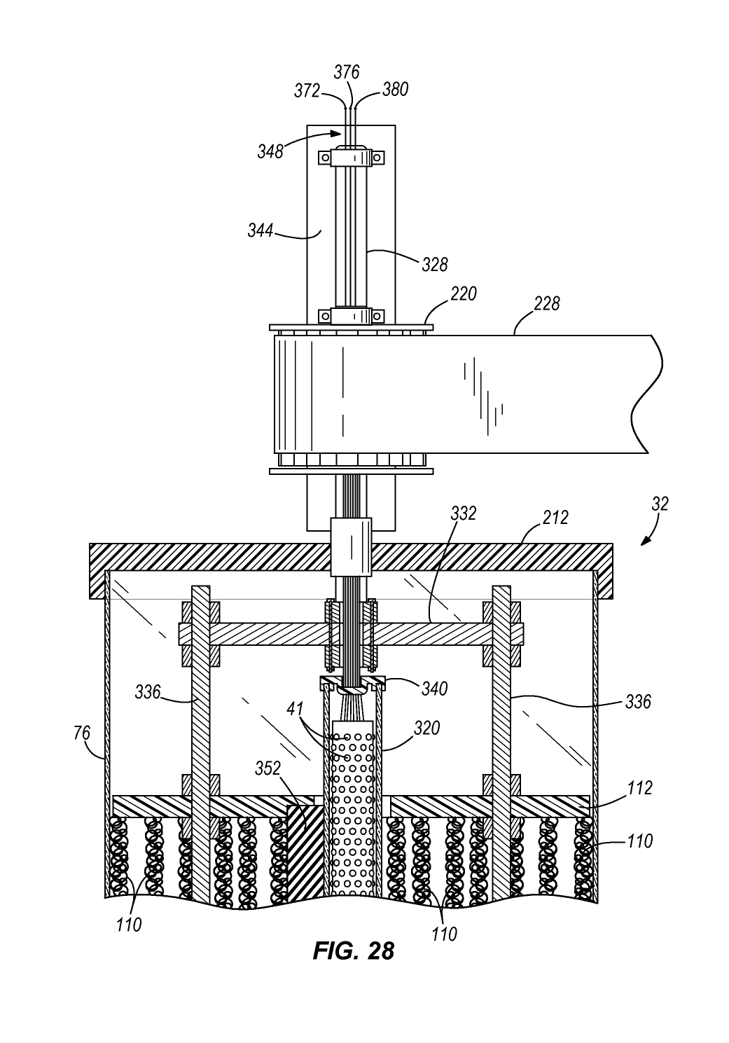

Referring now to FIGS. 27 and 28, another exemplary embodiment of an artificial light system 37 is shown. Components similar between the container and the artificial light system illustrated in FIGS. 25 and 26 and the container and the artificial light system illustrated in FIGS. 27 and 28 are identified by the same reference numbers.

In this illustrated exemplary embodiment, the artificial light system 37 includes a transparent or translucent hollow tube 320 positioned at or near a center of the container 32 and a light source 41, such as an array of light emitting diodes (LEDs), disposed within the tube 320. This artificial light system 37 provides light to the container 32 and algae from the inside-out, which is the opposite direction of sunlight 72 penetration into the container 32. The light from the artificial light system 37 may be used to supplement or substitute sunlight 72 and provides direct light to the interior of the container 32. In some instances, sunlight 72 penetration to the interior of the container 32 may be challenging because the sunlight 72 must penetrate through the housing 76, water, and algae disposed in the container 32 in order to reach the interior of the container 32.

The tube 320 is stationary relative to the housing 76 of the container 32 and the frame 108 rotates around the tube 320. A bottom end of the tube 320 extends through a central opening of the lower connector plate 116 and is secured to a central opening in the bushing 200. The central opening of the lower connector plate 116 is sufficiently large to provide a space between an interior edge of the opening and the tube 320. The second end of the tube 320 may be secured to the bushing 200 in a variety manners as long as the securement is rigid and does not allow movement between the tube 320 and the bushing 200 during operation. In some embodiments, an exterior wall of the tube 320 includes external threads and an interior edge of the bushing central opening includes complementary internal threads. In this embodiment, the tube threads into the bushing central opening and is threadably secured to the bushing 200. In other embodiments, the tube 320 may include threads on the exterior surface thereof, extend through the central opening of the lower connector plate 116 and one or more nuts or other threaded fasteners 324 may be threaded onto the tube 320 to secure the tube 320 to the bushing 200. In such an embodiment, a first nut 324 may be positioned above the bushing 200, a second nut 324 may be positioned below the bushing 200, and the nuts 324 may be tightened toward the bushing 200 to secure the tube 320 to the bushing 200. In still other embodiments, the bottom end of the tube 320 may be secured to the bushing 200 in a variety of other manners such as, for example, bonding, welding, adhering, or any other type of securement that prevents movement between the tube 320 and the bushing 200. A top end of the tube 320 extends through a central opening of the upper connector plate 112 with the central opening sufficiently large to provide a space between an interior edge of the central opening and the tube 320. The manner in which the top end of the tube 320 is supported will be described in greater detail below.

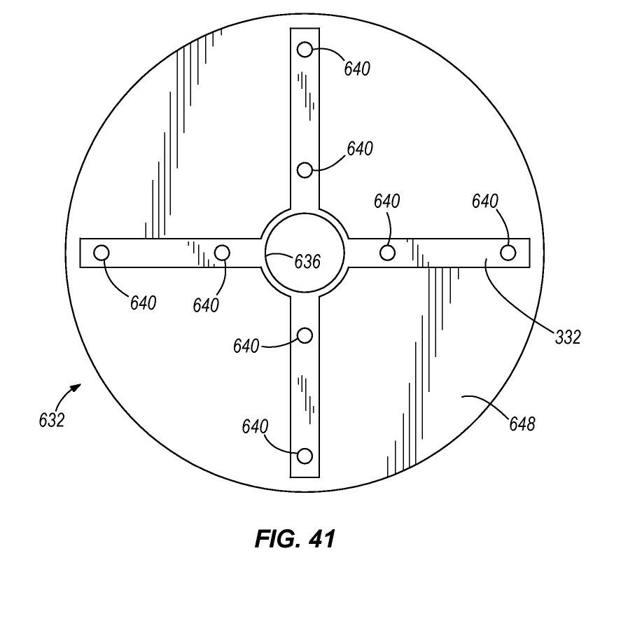

With continued reference to FIGS. 27 and 28, the frame 108 is required to have a different configuration since the artificial light system 37 includes the lighting tube 320 at the center of the container 32. In this illustrated exemplary embodiment, the frame 108 includes the upper and lower connector plates 112, 116, a hollow drive tube 328, a lateral support plate 332, and a plurality of support rods 336. The drive tube 328 is coupled to the pulley 220, drive belt 228, and motor 224, and is driven in a similar manner to the shaft 120. The lateral support plate 332 is secured to the drive tube 328 and rotates with the drive tube 328. The support plate 332 may be secured to the drive tube 328 in a variety of different manners as long as the support plate 332 and drive tube 328 rotate together. For example, the support plate 332 may be welded, bonded, adhered, threaded, or otherwise secured to the drive tube 328. The lateral support plate 332 may have a variety of different shapes and configurations including, for example, cylindrical, cross-shaped (see FIG. 41), etc. The plurality of support rods 336 are secured at their top ends to the support plate 332 and secured at their bottom ends to the lower connector plate 116. The support rods also pass through the upper connector plate 112 and may be secured thereto as well. In the illustrated exemplary embodiment, the frame 108 includes two support rods 336. However, the frame 108 may include any number of support rods 336 and still be within the spirit and scope of the present invention. During rotation of the frame 108, the motor 224 drives the belt 228 and pulley 220, which then rotate the drive tube 328. Rotation of the drive tube 328 rotates the support plate 332, thereby causing the support rods 336 to rotate and ultimately the upper and lower connector plates 112, 116 and the media 110.

With particular reference to FIG. 28, an exemplary manner for transferring electrical power to the LEDs 41 disposed in the tube 320 will be described. It is desirable that the interior of the tube 320 remain dry and absent from moisture to prevent damage to the LEDs 41 or other electronics of the system 20. In the illustrated exemplary embodiment, the top end of the tube 320 surrounds a bottom end of the drive tube 328 and a seal 340 is disposed between an exterior surface of the drive tube 328 and an interior surface of the tube 320, thereby creating an effective seal to prevent water from entering the tube 320. This sealing arrangement between the tube 320 and the drive tube 328 also provides support to the top end of the tube 320. A support device 344 may be provided around the drive tube 328 to provide additional support since the drive tube 328 is undergoing force exerted by the drive belt 228 and pulley 220. In order to provide electrical power to the LEDs 41 within the tube 320, a plurality of electrical wires 348 must run from an electrical power source to the LEDs 41. In the exemplary embodiment, the drive tube 328 is hollow and the electrical wires 348 extend into a top end of the drive tube 328, through the drive tube 328, out the bottom end of the drive tube 328, into the tube 320, and finally connect to the LEDs 41. As indicated above, the drive tube 328 rotates and the tube 320 and LEDs 41 do not rotate. Rotation of the electrical wires 348 would cause the wires 348 to twist and eventually break, disconnect from the LEDs 41, or otherwise interrupt the electrical power supply from the electrical power source to the LEDs 41. Accordingly, it is desirable for the electrical wires 348 to remain stationary within the drive tube 328 as the drive tube 328 rotates. This may be achieved in a variety of manners. For example, the electrical wires 348 may extend through a center of the drive tube 328 in a manner that does not cause contact between the wires 348 and an interior surface of the drive tube 328. By preventing contact between the wires 348 and the interior surface of the drive tube 328, the drive tube 328 will be able to rotate relative to the wires 348 without contacting the wires 348 and without twisting the wires 348. Also, for example, a secondary tube or device may be concentrically positioned within the drive tube 328, may be displaced inward from the interior surface of the drive tube 328, and may be stationary within the drive tube 328, thereby causing the drive tube 328 to rotate around the secondary tube or device. In such an example, the electrical wires 348 run through the secondary tube or device and are prevented from engaging the interior surface of the drive tube 328 by the secondary tube or device. Many other manners are contemplated for preventing twisting of the electrical wires 348 and are within the spirit and scope of the present invention.

With continued reference to FIG. 28, a wiper blade 352 is provided to contact and wipe against an outer surface of the tube 320. The wiper blade 352 is connected at its top end to the upper connector plate 112 and at its bottom end to the lower connector plate 116. Rotation of the frame 108 causes the wiper blade 352 to rotate, thereby causing the wiper blade 352 to wipe against the outer surface of the tube 320. This wiping clears any algae or other build-up attached to the outer surface of the tube 320. Having the tube 320 clear of algae and other build-up provides the tube 320 with optimum lighting performance. Significant algae build-up on the exterior surface of the tube 320 can adversely affect the effectiveness of the artificial light system 37 of this embodiment.

It should be understood that the artificial light system 37 illustrated in FIGS. 27 and 28 may be used on its own or in combination with any other artificial light system 37 disclosed herein. For example, the system 20 may include a first artificial light system 37 as illustrated in FIGS. 25 and 26 for illuminating the container 32 from the exterior and may include the artificial light system 37 illustrated in FIGS. 27 and 28 for illuminating the container 32 from the interior.

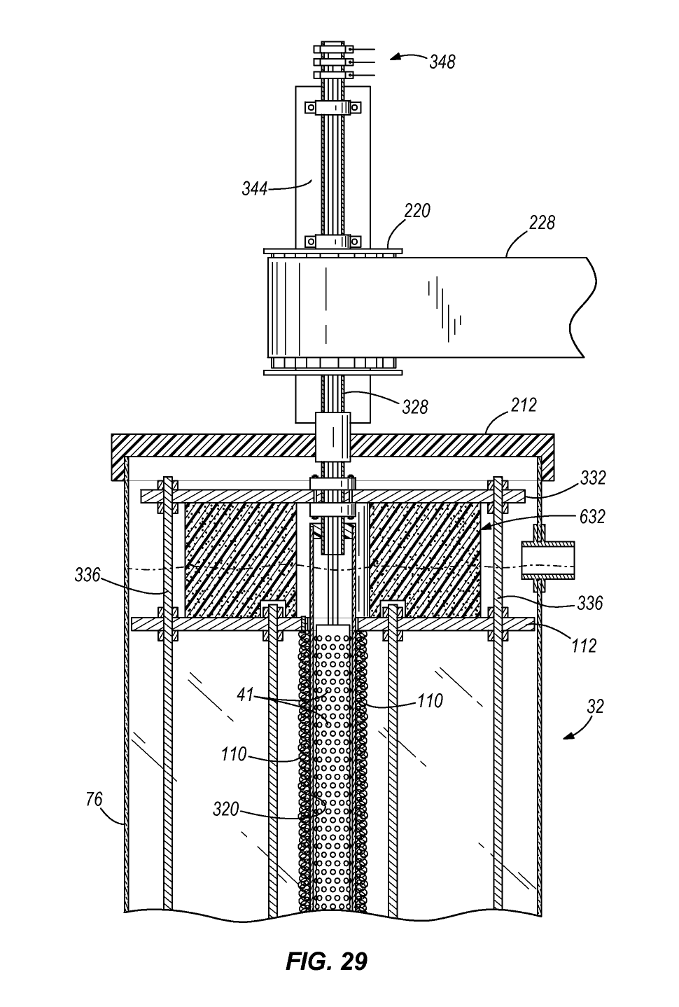

With reference to FIG. 29, an alternative manner of wiping the outer surface of the tube 320 is illustrated. In this illustrated exemplary embodiment, inner media segments or strands 110 are disposed adjacent to and engage the outer surface of the tube 320. Rotation of the frame 108 causes the media strands 110 to wipe against the outer surface of the tube 320 and clear algae or other debris from the outer surface of the tube 320. For purposes of simplicity, only the inner media strands 110 are illustrated in FIG. 29 even though other strands of media 110 would be present in the container 32.

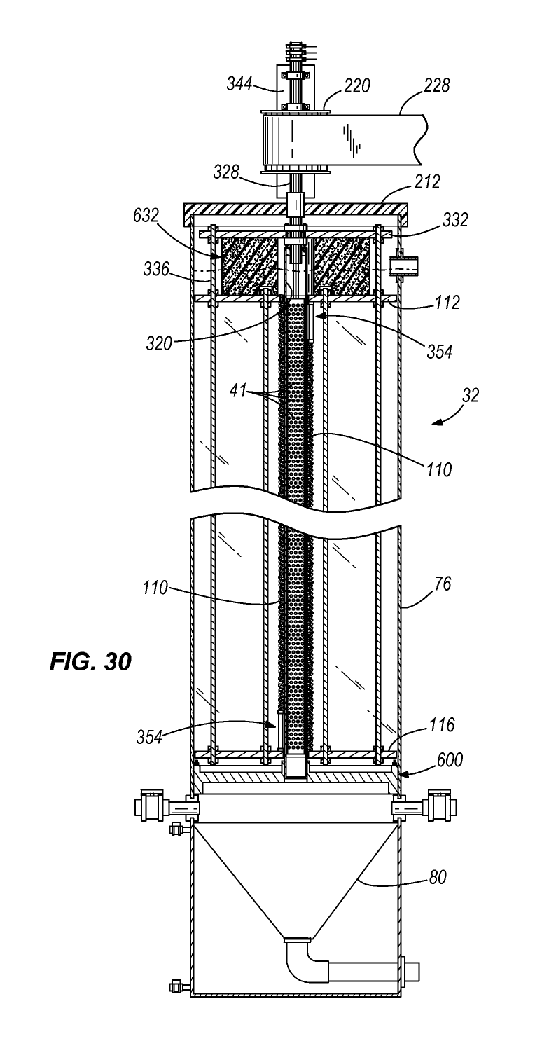

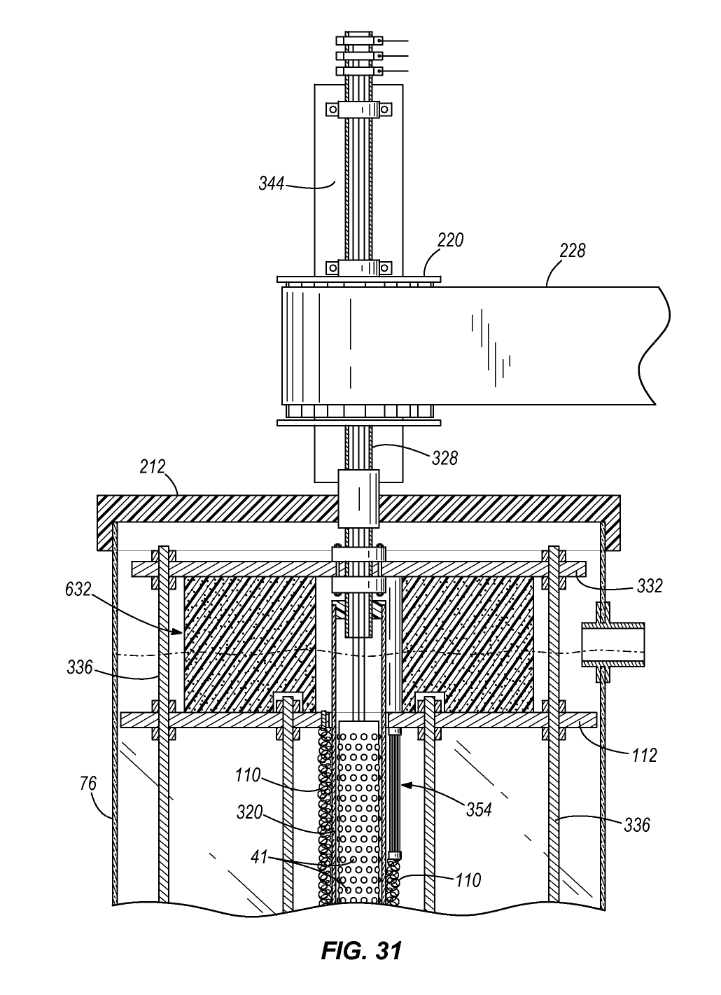

With reference to FIGS. 30 and 31, another alternative manner of wiping the outer surface of the tube 320 is illustrated. In this illustrated exemplary embodiment, the media strands 110 are positioned similarly to those illustrated in FIG. 29. That is, inner media strands 110 are positioned adjacent and in contact with the outer surface of the tube 320. Similar to FIG. 29, only the inner media strands 110 are illustrated in FIGS. 30 and 31 for simplicity even though other strands of media 110 would be present in the container 32. In some instances, rotation of the frame 108 may cause the inner media strands 110 to bow outward away from and out of contact with the outer surface of the tube 320 due to centrifugal force. To inhibit this outward bowing of the inner media strands 110, a rigid device 354 may be coupled to each of the inner media strands 110. The rigid devices 354 may be made of a variety of materials including, for example, plastic, metal, hard rubber, etc. Examples of rigid devices 354 that may be utilized include bungee cords, shock cords, plastic wire, metal wire, etc. The rigid devices 354 may extend the entire length of the inner media strands 110 between the upper and lower connector plates 112, 116 or may extend a portion of the length of the inner media strands 110. For example, the rigid devices 354 may extend downward from the upper connector plate 112, upward from the lower connector plate 116, or both downward from the upper connector plate 112 and upward from the lower connector plates 116, along only a portion of the inner media strands 110 such as, for example, six inches. With reference to the illustrated exemplary embodiment in FIGS. 30 and 31, a first rigid device 354 extends downward from the upper connector plate 112 a portion of the length of a first inner media strand 110 and a second rigid device 354 extends upward from the lower connector plate 116 a portion of the length of a second inner media strand 110. In this illustrated exemplary embodiment, the rigid devices 354 may not wipe against the outer surface of the tube 320. Accordingly, by offsetting the first and second rigid devices 354, the upper portion of the second inner media strand 110 will wipe the outer surface of the tube 320 in line with the first rigid device 354 and the bottom portion of the first inner media strand 110 will wipe against the outer surface of the tube 320 in line with the second rigid device 354. This arrangement insures that substantially the entire outer surface of the tube 320 will be wiped by inner media strands 110. Alternatively, the rigid devices 354 may be arranged to wipe against the outer surface of the tube 320.

Other alternatives for wiping the outer surface of the tube 320 are possible and are within the intended spirit and scope of the present invention.

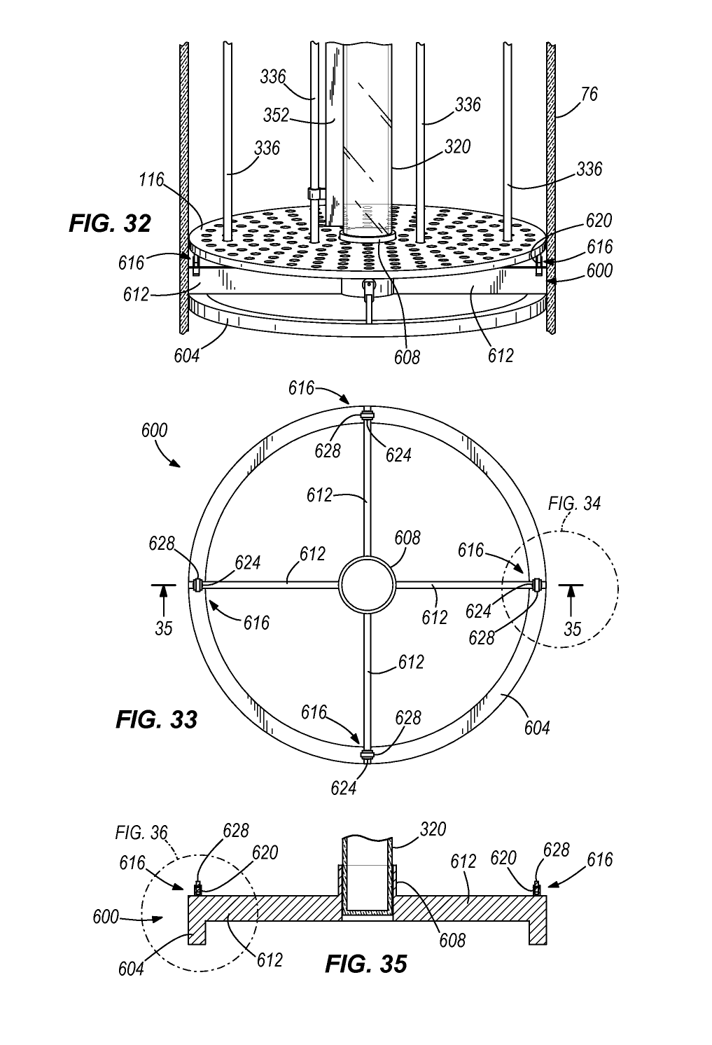

Referring now to FIGS. 32-37, an alternative manner for supporting the frame 108 and artificial light system 37 of FIGS. 27 and 28 is illustrated. In this illustrated exemplary embodiment, the system 20 includes a frame support device 600 having a circular support shelf 604, a central receptacle 608, a plurality of arms 612 extending from the central receptacle 608 toward the circular support shelf 604, and a plurality of roller devices 616 supported by the arms 612. The circular support shelf 604 is supported within the container housing 76 such that it is prevented from moving downward, thereby providing vertical support to the frame 108 resting thereon. The circular support shelf 604 may be supported within the housing 76 in a variety of different manners such as, for example, a press-fit, friction-fit, or interference fit, welding, fastening, adhering, bonding, or by an indentation or shelf extending from the inner surface of the housing 76 into the interior of the housing 76 upon which the circular support shelf 604 is supported, fastened, bonded, etc.

The central receptacle 608 is centrally located to receive a bottom end of the tube 320 and seal the bottom end of the tube 320 in a water tight manner, thereby preventing the ingress of water into the tube 320. The bottom end of the tube 320 may be coupled to the receptacle 608 in a variety of manners such as, for example, welding, fastening, adhering, bonding, press-fit, friction-fit, interference-fit, or other types of securement. In some embodiments, the coupling itself between the bottom end of the tube 320 and the receptacle 608 is sufficient to provide the water tight seal. In other embodiments, a sealing device such as, for example, a bushing, a water pump seal, an O-ring, packing material, etc., may be utilized to create the water tight seal between the bottom end of the tube 320 and the receptacle 608. In the illustrated exemplary embodiment, the frame support device 600 includes four arms 612. Alternatively, the frame support device 608 may include other quantities of arms 612 and be within the intended spirit and scope of the present invention. The arms 612 extend outward from the receptacle 608 and are supported from below on their distal ends by the support shelf 604. In some embodiments, the distal ends of the arms 612 are bonded, welded, adhered, otherwise secured to, or unitarily formed with the support shelf 604. In other embodiments, the distal ends of the arms 612 may solely rest upon the support shelf 604 or be received in recesses defined in the shelf 604 to inhibit rotation of the of the arms 612 and the central receptacle 608. In the illustrated exemplary embodiment, a single roller device 616 is secured to a top of each of the distal ends of the arms 612. The roller devices 616 include a base 620, an axle 624, and a roller 628 rotatably supported by the axle 624. The axles 624 are parallel to the arms 612 and the rollers 628 are oriented perpendicularly to the axles 624 and arms 612. The roller devices 616 are positioned to engage a bottom surface of the lower connector plate 116 and allow the lower connector plate 116 to roll over and relative to the frame support device 600. In this manner, the frame support device 600 provides vertical support to the frame 108 and allows the frame 108 to rotate relative to the frame support device 600. It should be understood that the frame support device 600 may include other numbers of roller devices 616 oriented in other manners such as, for example, multiple roller devices 616 per arm 612, roller devices 616 positioned on less than all the arms 612, roller devices 616 positioned on alternating arms 612, etc. It should also be understood that other devices may be used in place of the roller devices 616 to facilitate movement of the lower connector plate 116 relative to the frame support device 600, while providing vertical support to the frame 108.

It should further be understood that a frame support device 600 may also be utilized with the upper connector plate 112. In such an instance, the upper frame support device 600 would be positioned directly underneath the upper connector plate 112, engage the bottom surface of the upper connector plate 112 to provide vertical support, and allow rotation of upper connector plate 112 relative to the upper frame support device 600. Such an upper frame support device 600 may be configured and may function in much the same manner as the lower frame support device 600.

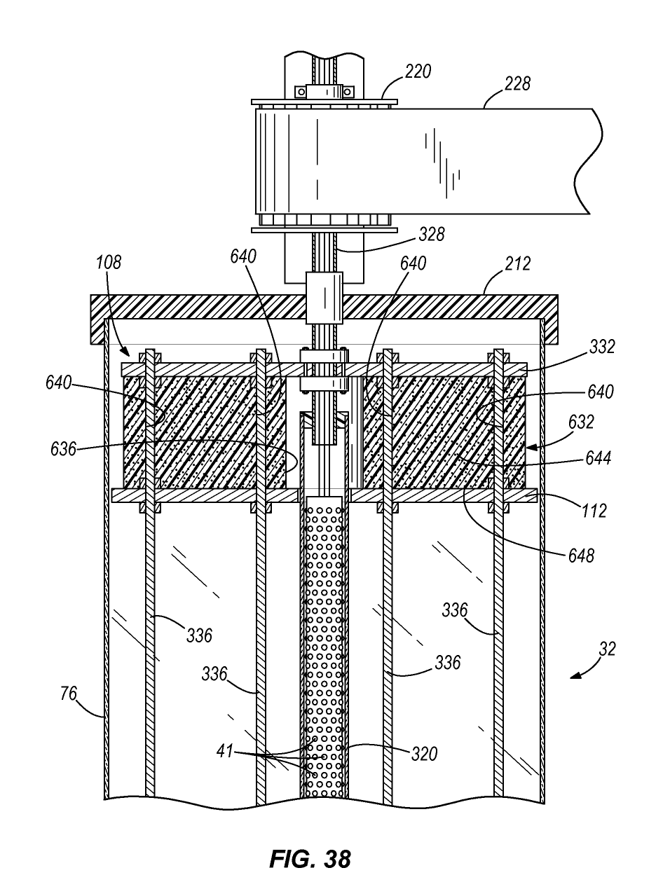

With reference to FIGS. 38-41, yet another alternative manner for supporting the frame 108 and artificial light system 37 of FIGS. 27 and 28 is illustrated. In this illustrated exemplary embodiment, the system 20 includes a float device 632 for providing vertical support to the frame 108. In some exemplary embodiments, the float device 632 may provide a portion of the vertical support required to maintain the frame 108 in the desired position. In other exemplary embodiments, the float device 632 may provide the entire vertical support required to maintain the frame 108 in the desired position. The float device 632 is positioned between the lateral support plate 332 and the upper connector plate 112. In other embodiments, the float device 632 may be positioned under the upper connector plate 112 or under the lower connector plate 116. Also, in further embodiments, the system 20 may include a plurality of float devices 632 such as, for example, two float devices 632. In such an exemplary embodiment, a first float device may be positioned between the lateral support plate 332 and upper connector plate 112 as illustrated in FIG. 38 and a second float device may be positioned under the lower connector plate 116.

The float device 632 may have any shape and configuration as long as it provides a desired amount of vertical support to the frame 108 disposed within the container 32. In the illustrated exemplary embodiment, the float device 632 is substantially cylindrical in shape to compliment the shape of the container housing 76. The thickness or height of the float device 632 may vary depending on the amount of buoyancy desired. The float device 632 includes a central opening 636 for allowing the drive tube 328 and the tube 320 to pass therethrough, and a plurality of openings 640 for allowing support rods 336 to pass through the float device 632. As indicated above, the container 32 may include any number and any configuration of support rods 336 and, similarly, the float device 632 may include any number and any configuration of openings 640 to accommodate the total number of support rods 336.

The float device 632 may be comprised of a wide variety of buoyant materials. In some exemplary embodiments, the float device 632 is comprised of a closed cell material that inhibits absorption of water. In such embodiments, the float device 632 may be comprised of a single closed cell material or multiple closed cell materials. Exemplary closed cell materials that the float device 632 may be comprised of include, but are not limited to, polyethylene, neoprene, PVC, and various rubber blends. In other exemplary embodiments, the float device 632 may be comprised of a core 644 and an outer housing 648 surrounding and enclosing the core 644. The core 644 may be comprised of a closed cell material or an open cell material, while the outer housing 648 is preferably comprised of a closed cell material due to its direct contact with water in the container 32. In instances where the core 644 is closed cell material and does not absorb water, the outer housing 648 may be water and air tight or may not be water and air tight. In instances where the core 644 is open cell material, the outer housing 648 is preferably water and air tight around the core 644 to inhibit water from accessing the core 644 and being absorbed by the core 644. Exemplary closed cell materials that the core 644 may be comprised of include, but are not limited to, polyethylene, neoprene, PVC, and various rubber blends, and exemplary open cell materials that the core 644 may be comprised of include, but are not limited to, polystyrene, polyether, and polyester polyurethane foams. Exemplary materials that the outer housing 648 may be comprised of include, but are not limited to, fiberglass re-enforced plastic, PVC, rubber, epoxy, and other water proof coated formed shells.

With particular reference to FIG. 41, the float device 632 is illustrated with an exemplary lateral support plate 332. In this illustrated exemplary embodiment, the lateral support plate 332 is substantially cross-shaped. One exemplary reason for providing a cross-shaped lateral support plate 332 is to reduce the amount of material and the overall weight of the lateral support plate 332. By reducing the weight of the lateral support plate 332, the overall frame 108 weighs less and the float device 632 is required to support less weight. In this exemplary cross-shaped embodiment, the material of the lateral support plate 332 is removed between locations where the support rods 336 connect to the lateral support plate 332. As indicated above, the container 32 may include any number and any configuration of support rods 336 and, similarly, the lateral support plate 332 may have any configuration to accommodate the number and configuration of support rods 336.

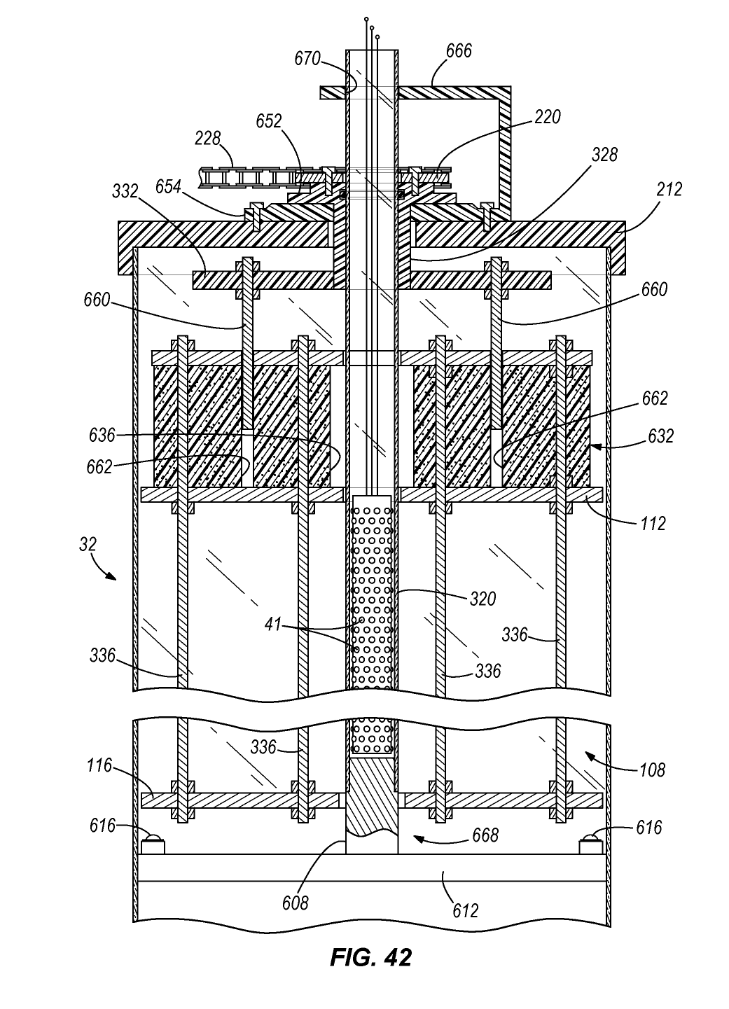

Referring now to FIGS. 42-45, another exemplary embodiment of the container 32 is illustrated. In this exemplary embodiment, the container 32 includes an alternative drive mechanism for rotating the frame 108 and media 110. In the illustrated embodiment, the drive mechanism includes a motor (not shown), a drive chain 228, a sprocket or gear 220, a plate 652 coupled to the gear 220, a centering ring 654 encircling the plate 652 to ensure that plate 652 remains centered, and a drive tube 328 coupled to the plate 652. The motor drives the chain 228 in a desired direction, thereby rotating the gear 220. Since the gear 220 is coupled to the plate 652 and the plate 652 is coupled to the drive tube 328, rotation of the gear 220 ultimately rotates the drive tube 328. The tube 320 is fixed-in-place in the center of the container 32 and the gear 220, plate 652, centering ring 654, and drive tube 328 all encircle and rotate around the central tube 320. A sealing member 656 such as, for example, an O-ring is disposed in a recess 658 defined in the gear 220, encircles the tube 320, and engages an exterior surface of the tube 320 to seal around the tube 320. The sealing member 656 inhibits liquid within the container 32 from leaking out of the container 32 between the tube 320 and the drive mechanism. Alternatively, the sealing member 656 may be disposed in a recess defined in other components of the drive mechanism such as, for example, the plate 652, the drive tube 328, etc., and may engage the exterior surface of the tube 320 to seal around the tube 320.

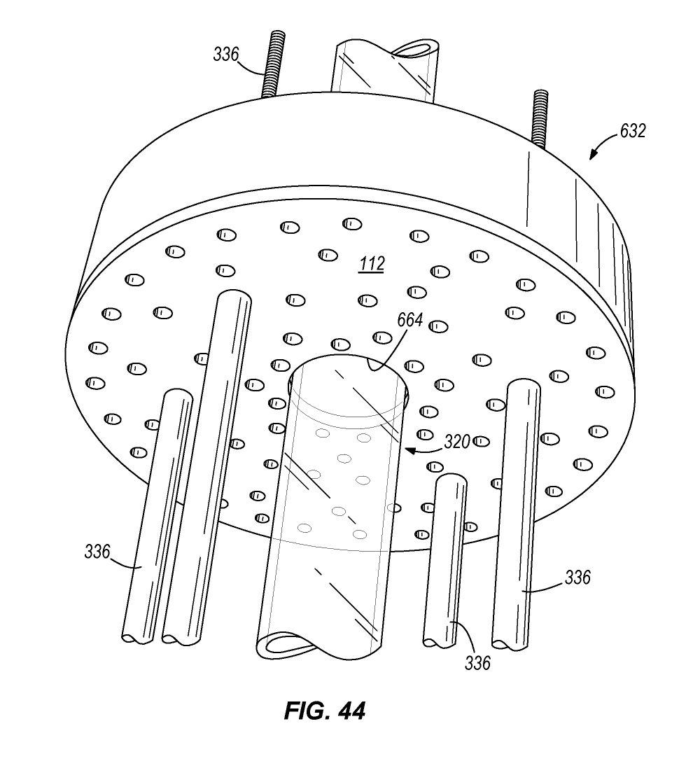

With particular reference to FIG. 42, the drive mechanism also includes a support plate 332 coupled to and rotatable with the drive tube 328. Extending downward from the support plate 332 are two dowels 660 that insert into apertures 662 defined in the float device 632. The dowels 660 couple the drive mechanism to the float device 632 such that rotation of the drive mechanism facilitates rotation of the float device 632 and the frame 108. However, vertical movement of the float device 632 relative to the dowels 660 is uninhibited. Such vertical movement of the float device 632 occurs as the level of water changes within the container 32. Referring to FIG. 44, the float device 632 includes a central opening 636 through which the tube 320 extends. The central opening 636 is sufficiently sized to allow the float device 632 to rotate relative to the tube 320 without significant friction between the exterior surface of the tube 320 and the float device 632. While the exemplary illustrated embodiment includes two dowels 660, any number of dowels 660 may be used to couple the drive mechanism to the float device 632. In addition, the drive mechanism may be coupled to the frame 108 in manners other than the illustrated configuration of the dowels 660 and float device 632.

As indicated above, the tube 320 is fixed in place and does not rotate. Referring now to FIGS. 42-45, the container 32 includes a first support 666 secured to cover 212 for supporting the top of the tube 320 and a second support 668 for supporting the bottom of the tube 320. The top support 666 includes an aperture 670 in which the top of the tube 320 is positioned. The aperture 670 is adequately sized to tightly engage the exterior surface of the tube 320 to inhibit movement of the top of the tube 320 relative to the top support 666. The bottom support 668 includes a central receptacle 608, a plurality of arms 612 extending from the central receptacle 608, and a plurality of roller devices 616 supported by the arms 612. The tube 320 is rigidly secured to the central receptacle 608 to inhibit movement between the tube 320 and the receptacle 608. The arms 612 include a curved plate 672 at their ends to engage the interior surface of the container 32 to inhibit substantial lateral movement of the bottom support 668 relative to the container housing 76. Since the frame 108 is lifted within the container 32 due to buoyancy of the float device 632 on the water, drainage of the water from the container 32 causes the frame 108 to lower in the container 32 until the lower connector plate 116 rests upon the roller devices 616. If rotation of the frame 108 is desired while water is drained from the container 32, the roller devices 616 facilitate such rotation. In the illustrated embodiment, the bottom support 668 includes four roller devices 616. In other embodiments, the bottom support 668 may include any number of roller devices 616 to accommodate rotation of the frame 108. The bottom support 668 may be made of stainless steel or other relatively dense material to provide the bottom support 668 with a relatively heavy weight, which counteracts buoyant forces exerted upwardly to the tube 320 when the container 32 is filled with water. The relatively heavy weight of the bottom support 668 also facilitates insertion of the internal components of the container 32 into a water filled container 32. Such internal components may include, for example, the bottom support 668, the tube 320, the frame 108, the media 110, and a portion of the drive mechanism.

The tube 320 described in connection with the exemplary embodiment illustrated in FIGS. 42-45 is capable of having the same functionality as any of the other tubes 320 described and illustrated in the other tube embodiments. For example, the tube 320 of this embodiment is capable of containing lighting elements similar to those illustrated in FIGS. 27 and 28-38.

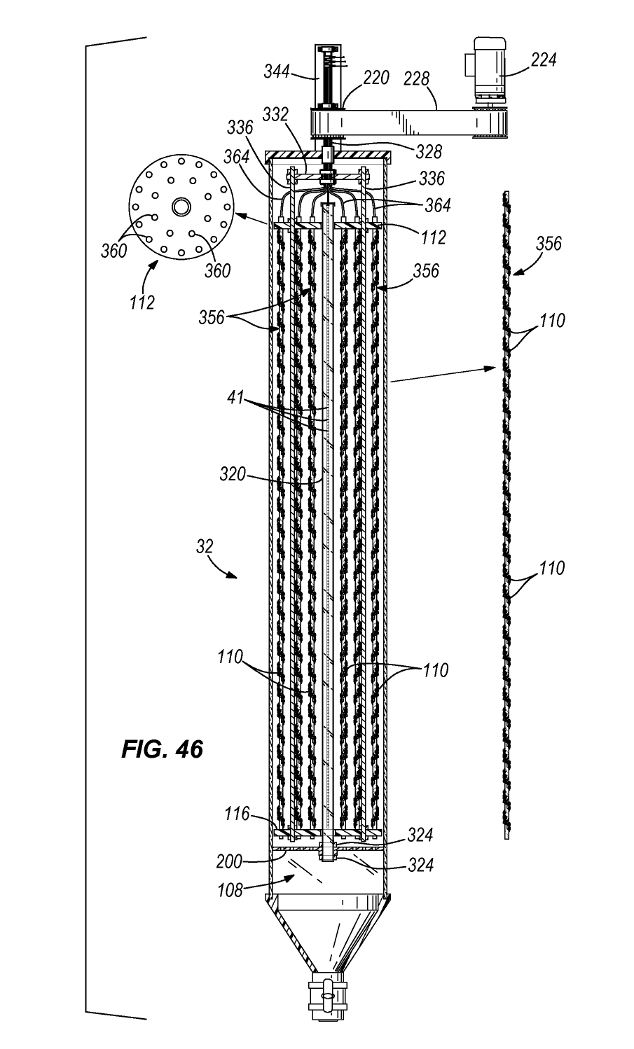

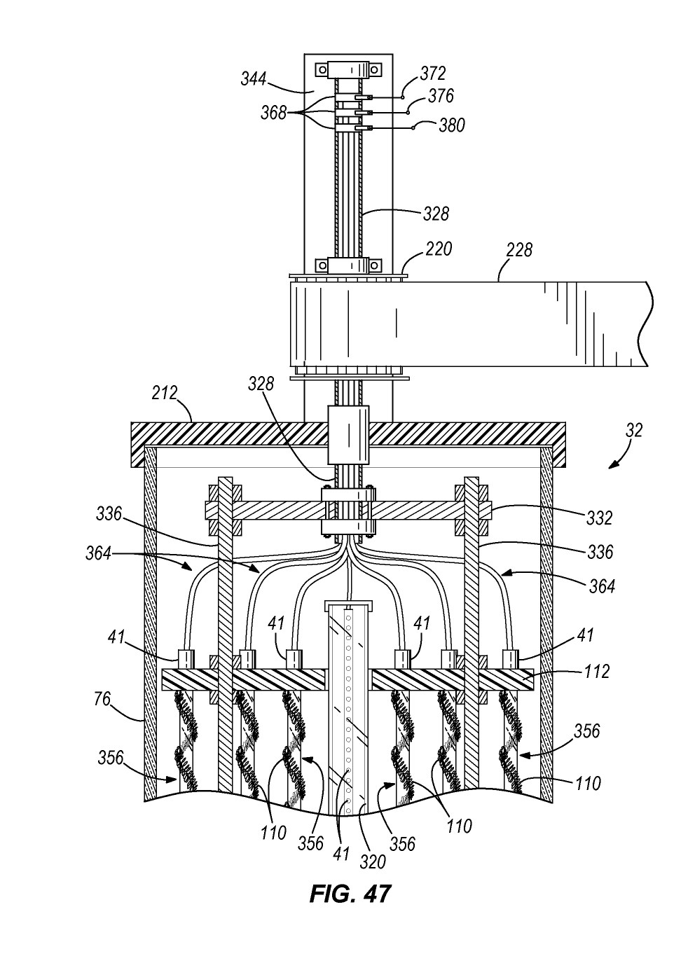

Referring now to FIGS. 46 and 47, yet another exemplary embodiment of an artificial light system 37 is shown. Components similar between the container and the artificial light systems illustrated in FIGS. 25-28 and the container and the artificial light system illustrated in FIGS. 46 and 47 are identified by the same reference numbers.

The artificial light system 37 illustrated in FIGS. 46 and 47 may either include a central tube 320 and associated light source 41 similar to the tube 320 and light source illustrated in FIGS. 27 and 28 (see FIG. 46) or the artificial light system 37 may not include the tube 320 and light source illustrated in FIGS. 27 and 28 (see FIG. 47). In the embodiment of the artificial light system 37 illustrated in FIG. 46 including the tube 320 and light source 41, the tube 320 and light source 41 are similar to the tube 320 and light source 41 illustrated in FIGS. 27 and 28.

With continued reference to FIGS. 46 and 47, the artificial light system 37 includes a plurality of light elements 356 connected between upper and lower connector plates 112, 116. The light elements 356 are capable of emitting light within the container 32. In the illustrated exemplary embodiment, the light elements 356 are cylindrically shaped rods made of a material that easily emits light such as, for example, glass, acrylic, etc. Alternatively, the light elements 356 may have other shapes and be made of other materials, and such illustrated and described examples are not intended to be limiting. In some exemplary embodiments, the material that comprises the light elements 356 includes an infrared inhibitor or infrared filter applied to the light elements 356 or included in the composition of the light element material in order to reduce or limit the heat build-up that occurs in the light elements 356 as light passes therethrough. The light elements 356 are connected at their ends respectively to the upper and lower connector plates 112, 116, which are configured to include a hole 360 for receiving an end of each lighting element 356 (see top view of upper connector plate 112 in FIG. 46). The artificial light system 37 may include any number of light elements 356 and the upper and lower connector plates 112, 116, may include a complementary number of holes 360 therein to accommodate the ends of the light elements 356. One or more media strands 110 is/are wrapped around each of the light elements 356 to bring the media 110 into close proximity with the light elements 356. Since the light elements 356 are secured to the upper and lower connector plates 112, 116, the light elements 356 rotate with the frame 108.

With particular reference to FIG. 47, the artificial light system 20 includes a plurality of light sources 41, one associated with each of the light elements 356, for providing light to the light elements 356. In the illustrated exemplary embodiment, the light sources 41 are LEDs. In other embodiments, the light sources 41 may be other types of lights and still be within the spirit and scope of the present invention. The light sources 41 are preferably contained within a water proof housing or are otherwise sealed to prevent water from penetrating into the light sources 41. The light sources 41 are positioned at and emit light into the top end of the light elements 356. Light emitted into the light elements 356 travels through the light elements 356, emits from the light elements 356 into the container 32, and onto the media 110 and algae. Alternatively, the light sources 41 may be positioned at other locations of the light elements 356 such as, for example, the bottom end or intermediary positions between the top and bottom ends, to emit light into the light elements 356.

Electrical power is supplied to the light sources 41 from an electrical power source via electrical wires 364. As indicated above, the light elements 356 rotate with the frame 108. Accordingly, electrical power needs to be supplied to the light sources 41 without twisting the electrical wires 364. Similar to the embodiment of the artificial light system 37 illustrated in FIGS. 27 and 28, the present exemplary embodiment of the artificial light system 37 includes a hollow drive tube 328. The drive tube 328 transfers the rotational force exerted from the motor 224 ultimately to the frame 108. In the present exemplary embodiment, the electrical wires 364 must rotate with the light sources 41 to prevent the electrical wires 364 from twisting. Accordingly, the drive tube 328, electrical wires 364, and frame 108 all rotate together. Continual, uninterrupted electrical power is required to be supplied to the electrical wires 364 connected to the light sources 41 in order to ensure uninterrupted operation of the light sources 41. This continual, uninterrupted electrical power may be provided to the light sources 41 in a variety of different manners and the illustrated and described exemplary embodiments are not intended to be limiting. In the illustrated exemplary embodiment, the artificial light system 37 includes a plurality of copper rings 368 fixed to an exterior surface of the drive tube 328, one ring for engaging each of a positive contact 372, a negative contact 376, and a ground contact 380. The copper rings 368 are isolated from one another to prevent a short circuit from occurring. The positive and negative contacts 372, 376 are coupled to the electrical source and the ground contact 380 is coupled to a ground, and each contact 372, 376, 380 engages an outer surface of a respective ring 368. The contacts 372, 376, 380 are biased toward the rings 368 to ensure continual engagement between the contacts 372, 376, 380 and the rings 368. As the drive tube 328 and rings 368 rotate, the rings 368 move under the contacts 372, 376, 380 and the contacts 372, 376, 380 slide along the exterior surface of the rings 368. The biasing of the contacts 372, 376, 380 toward the rings 368 ensures that the contacts 372, 376, 380 will continually engage the rings 368 during movement. Other manners of providing continual, uninterrupted electrical power to the light sources 41 are contemplated and are within the spirit and scope of the present invention.

In some exemplary embodiments of the artificial light system 37 illustrated in FIGS. 46 and 47, the light elements 356 have a smooth or polished exterior surface. In other exemplary embodiments, the light elements 356 have a scratched, chipped, dented, or otherwise imperfect exterior surface to assist with diffraction of the light from the interior of the light elements 356 to the exterior of the light elements 356. In yet other exemplary embodiments, the light elements 356 may be formed in a shape promoting diffraction of the light from the interior of the light elements 356 to the exterior of the light elements 356.

It should be understood that the artificial light system 37 illustrated in FIGS. 46 and 47 may be used on its own or in combination with any other artificial light system 37 disclosed herein. For example, the system 20 may include a first artificial light system 37 as illustrated in FIGS. 25 and 26 for illuminating the container 32 from the exterior and may include the artificial light system 37 illustrated in FIGS. 46 and 47 for illuminating the container 32 from the interior.

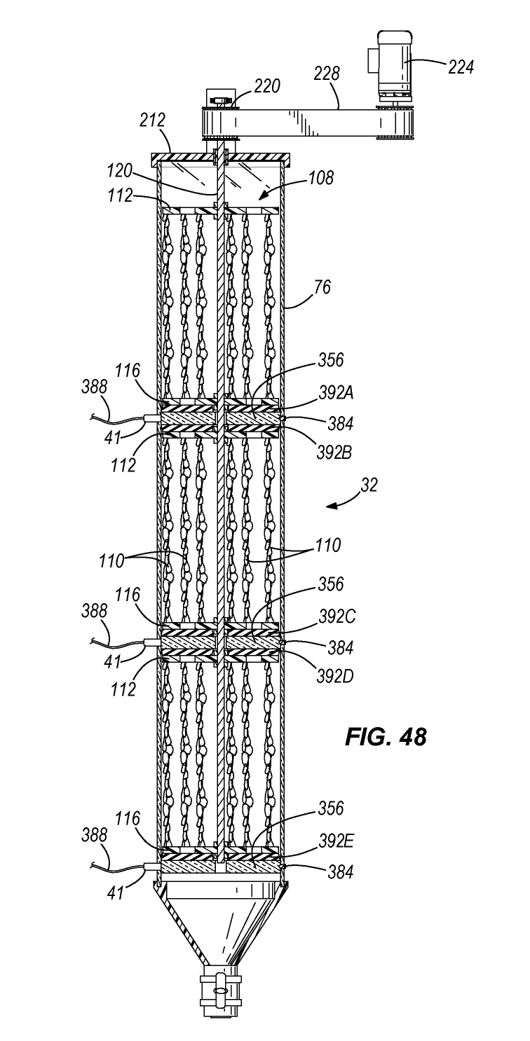

Referring now to FIG. 48, a further exemplary embodiment of an artificial light system 37 is shown. Components similar between the container and the artificial light systems illustrated in FIGS. 25-47 and the container and the artificial light system illustrated in FIG. 48 are identified by the same reference numbers.

This artificial light system 37 includes a plurality of light elements 356 disposed at various heights along the container 32. The light elements 356 are capable of emitting light within the container 32. In the illustrated exemplary embodiment, the light elements 356 are cylindrically shaped discs made of a material that easily emits light such as, for example, glass, acrylic, etc. Alternatively, the light elements 356 may have other shapes and may be made of other materials, and such illustrated and described examples are not intended to be limiting. In the illustrated exemplary embodiment, the artificial light system 37 includes three light elements 356, however, the number of light elements 356 illustrated in this embodiment is for illustrative purposes and is not intended to be limited. The system 37 may include any number of light elements 356 and still be within the spirit and scope of the present invention. The light elements 356 are secured in place within the container 32 and do not move relative to the container 32. In the illustrated exemplary embodiment, the light elements 356 are secured in place by friction stops 384, one for each lighting element 356. Alternatively, the light elements 356 may be secured in place by any number of friction stops 384 and by other manners of securement. For example, the light elements 356 may be secured in place in the container 32 by a friction-fit or press-fit, fasteners, bonding, adhering, welding, or any other manner of securement. The light elements 356 are generally round in shape and have a similar diameter to the diameter of the container 32. The artificial light system 37 also includes a plurality of light sources 41, at least one light source 41 for each lighting element 356, providing light to the light elements 356. The light sources 41 may be a variety of different types of light sources including, for example, LEDs, fluorescents, light conducting fibers, etc. The light sources 41 are positioned to emit light into or onto the light elements 356 and the light elements 356 then emit light into the container 32. The light sources 41 are coupled to electrical power via electrical wires 388.

Since the light elements 356 are stationary and essentially divide the container 32 into sections (three sections in the illustrated exemplary embodiment), the frame 108 and media 110 must be altered to accommodate such sections. Rather than the frame 108 including a single upper connector plate 112 and a single lower connector plate 116, the frame includes upper and lower connector plates 112, 116 for each section. More particularly, the frame 108 includes six total connector plates comprised of three upper connector plates 112 and three lower connector plates 116. Media 110 is strung between each set of upper and lower connector plates 112, 116 in any of the manners described herein. Accordingly, the media 110 is specific to each individual section (i.e., media present in the top section is not strung to the second or third section, and vice versa).

With continued reference to FIG. 48, the frame 108 is rotated in a similar manner to that described above in connection with the frame 108 illustrated in FIGS. 3 and 4. Accordingly, the shaft 120 rotates the connector plates 112, 116 and media 110 in each section. A plurality of wipers 392 are secured to the connector plates 112, 116 and wipe against an exterior surface of the light elements 356 to assist with cleaning the exterior surface and enhancing light emission from the light elements 356. The wipers 392 are secured to surfaces of the connector plates 112, 116 adjacent top and bottom surfaces of the light elements 356. In the illustrated exemplary embodiment, a first wiper 392A is secured to a bottom surface of the lower connector plate 116 in the top section of the container 32, a second wiper 392B is secured to a top surface of the upper connector plate 112 in the middle section, a third wiper 392C is secured to a bottom surface of the lower connector plate 116 in the middle section, a fourth wiper 392D is secured to a top surface of the upper connector plate 112 in the bottom section, and a fifth wiper 392E is secured to a bottom surface of the lower connector plate 116 in the bottom section. With this configuration of wipers 392, the necessary exterior surfaces of the light elements 356 are wiped and cleaned to enhance light emission into the container 32. The wipers 392 may be made of a variety of different materials such as, for example, rubber, plastic, and other materials.

Similar to the light elements 356 described above with reference to FIGS. 46 and 47, the light elements 356 illustrated in FIG. 48 may have a smooth or polished exterior surface, or a scratched, chipped, dented, or otherwise imperfect exterior surface to assist with diffraction of the light from the interior of the light elements 356 to the exterior of the light elements 356. Additionally, the light elements 356 may be formed in a shape promoting diffraction of the light from the interior of the light elements 356 to the exterior of the light elements 356.

It should be understood that the artificial light system 37 illustrated in FIG. 48 may be used on its own or in combination with any other artificial light system 37 disclosed herein. For example, the system 20 may include a first artificial light system 37 as illustrated in FIGS. 25 and 26 for illuminating the container 32 from the exterior and may include the artificial light system 37 illustrated in FIG. 48 for illuminating the container 32 from the interior.

Referring now to FIG. 49, an exemplary embodiment of the flushing system 38 is shown. This exemplary flushing system 38 is one of many types of flushing systems contemplated and is not intended to be limiting. The exemplary flushing system 38 is operable to assist with removing algae from the media 110 or for cleaning the interior of the container 32 in the event an invasive species or other contaminant has infiltrated the container 32. The flushing system 38 allows the interior of the container 32 to be rinsed or cleaned without disassembling the container 32 or other components of the system 20. The exemplary flushing system 38 includes a pressurized water source (not shown), a pressurized water inlet tube 42 in fluid communication with the pressurized water source, and a plurality of spray nozzles 43 in fluid communication with the tube 42. The spray nozzles 43 are incrementally disposed along the height of the container housing 76 at any desired spacing and are positioned in holes or cutouts in the container housing 76. An air and water tight seal is created between each of the spray nozzles 43 and the associated hole to prevent air and water from leaking into or from the container 32. In some embodiments, the spray nozzles 43 are positioned in the holes such that tips of the spray nozzles 43 are flush with or recessed from the interior surfaces 196 of the container housings 76 such that the nozzles 43 do not protrude into the container housings 76. This ensures that the media 110, when rotated, does not engage the spray nozzles 43. Operation of the flushing system 38 will be described in greater detail below.

While the containers 32 are cultivating algae, it is important that the containers 32 maintain an environment beneficial to the growth of the algae. One environmental parameter paramount to the growth of the algae is the water temperature in which the algae is located. The containers 32 must maintain the water therein within a particular temperature range that promotes efficient algae growth. Appropriate temperature ranges may depend on the type of algae being cultivated within the containers 32. For example, the water temperature within the containers 32 should remain as close to 20° C. as possible and not exceed 35° C. when the algae species P. Tricornutum is cultivated within the containers 32. The present example is one of many various temperature ranges in which the water within the containers 32 is controlled to promote effective algae cultivation and is not intended to be limiting. The water is capable of being controlled within different temperature ranges for different types of algae.

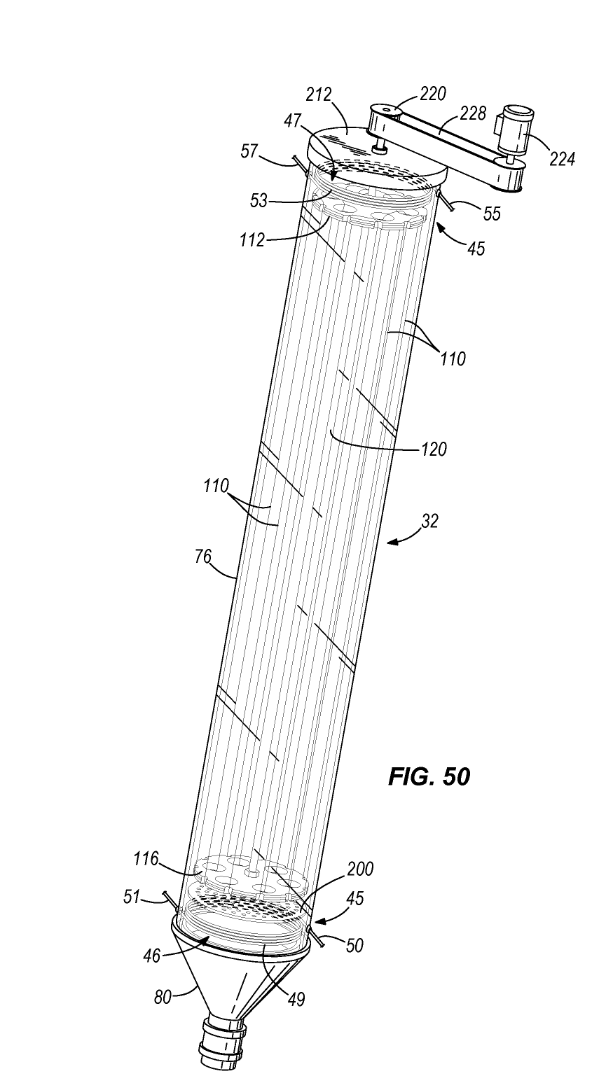

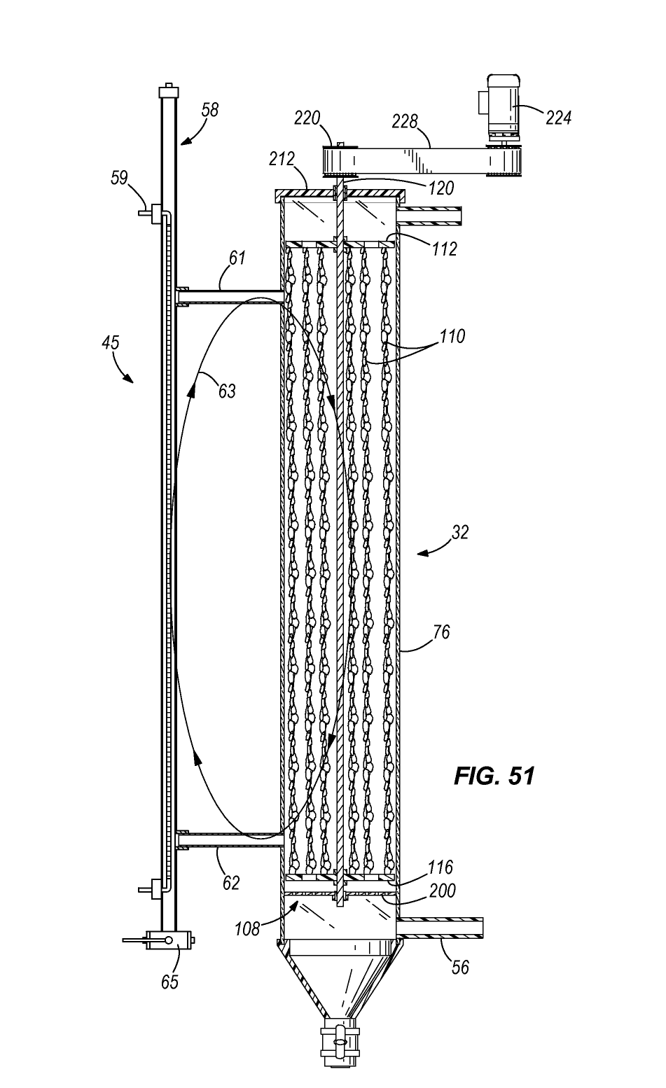

A variety of different temperature control systems can be utilized to assist with controlling the water temperature within the containers 32. With reference to FIGS. 50 and 51, two exemplary temperature control systems 45 are illustrated and will be described herein. These exemplary temperature control systems 45 are two of many types of temperature control systems 45 contemplated and are not intended to be limiting.

With particular reference to FIG. 50, a single container 32 and an associated temperature control system 45 is illustrated. The temperature control system 45 associated with each container 32 is substantially identical and, therefore, only a single temperature control system 45 will be illustrated and described. The temperature control system 45 includes a heating portion 46 and a cooling portion 47. The heating portion 46 heats the water when necessary and the cooling portion 47 cools the water when necessary. The heating portion 46 is disposed within and near a bottom of the container 32. This orientation of the heating portion 46 takes advantage of the natural thermal laws whereas heat always rises. Accordingly, when the heating portion 46 is activated, water heated by the heating portion 46 rises up through the container 32 and pushes the cooler water down toward the heating portion 46 where the cooler water is heated. The cooling portion 47 is disposed within and near a top of the container 32. Similarly, this orientation of the cooling portion 47 also takes advantage of the natural thermal laws. Accordingly, when the cooling portion 47 is activated, water cooled by the cooling portion 47 is displaced by rising water having a higher temperature than the cooled water. Displacement of the cooled water causes the cooled water to move downward in the container 32.

The heating portion 46 includes a heating coil 49, a fluid inlet 50, and a fluid outlet 51. The inlet 50 and outlet 51 respectively allow the introduction and exhaustion of fluid into and out of the heating coil 49. The fluid introduced into the heating coil 49 through the inlet 50 has an elevated temperature compared to the temperature of the water disposed within the container 32 in order to heat the water within the container 32. The fluid can be a variety of different types of fluids including, but not limited to, liquids, such as water, and gases. The cooling portion 47 includes a cooling coil 53, a fluid inlet 55, and a fluid outlet 57. The inlet 55 and outlet 57 respectively allow the introduction and exhaustion of fluid into and out of the cooling coil 53. The fluid introduced into the cooling coil 53 through the inlet 55 has a lower temperature than the temperature of the water disposed within the container 32 in order to cool the water within the container 32. The fluid can be a variety of different types of fluids including, but not limited to, liquids, such as water, and gases.

Referring now to FIG. 51, an alternative example of the temperature control system 45 is illustrated. Similar to the example illustrated in FIG. 50, a single container 32 and an associated temperature control system 45 is illustrated. The temperature control system 45 associated with each container 32 is substantially identical and, therefore, only a single temperature control system 45 will be illustrated and described. The temperature control system 45 includes an insulated riser pipe 58 and an exchanger tube 59 passing into and through the insulated riser pipe 58. The insulated riser pipe 58 is in fluid communication with the container 32 through an upper transfer pipe 61 and a lower transfer pipe 62. Water from the container 32 is within the riser pipe 58 and the upper and lower transfer pipes 61, 62. If the temperature of the water within the container 32 requires cooling, a fluid cooler than the temperature of the water within the container 32 is passed through the exchanger tube 59. The water within the riser pipe 58 surrounds the exchanger tube 59 and is cooled. The cooled water within the riser pipe 58 is displaced by warmer water within the container 32, thereby causing a counter-clockwise circulation of water within the container 32 and the riser pipe 58. In other words, the cooled water moves downward in the riser pipe 58, and into the bottom of the container 32 through the lower transfer pipe 62, while the warmer water within the container 32 moves out of the container 32, into the upper transfer pipe 61, and into the riser pipe 58. If the temperature of the water within the container 32 requires heating, a fluid warmer than the temperature of the water within the container 32 is passed through the exchanger tube 59. The water within the riser pipe 58 surrounds the exchanger tube 59 and is warmed. The warmed water within the riser pipe 58 rises, thereby causing a clockwise circulation of the water (as represented by arrow 63) within the container 32 and the riser pipe 58. In other words, the warmed water moves upward in the riser pipe 58, and into the top of the container 32 through the upper transfer pipe 61, while the cooler water within the container 32 moves out of the container 32, into the lower transfer pipe 62, and into the riser pipe 58. In some embodiments, a more aggressive circulation of water is desired. In such embodiments, a sparger or air inlet 65 is positioned near the bottom of the riser pipe 58 to introduce air into the water located within the riser pipe 58. The introduction of air into the bottom of the riser pipe 58 causes the water within the riser pipe 58 to rise faster, thereby circulating the water through the riser pipe 58 and the container 32 at an increased rate. In some embodiments, a filter may be provided at junctions of the upper and lower transfer pipes 61, 62 and the container housing 76 to inhibit algae from entering the riser pipe 58 and potentially reducing flow capabilities or completely blocking the riser pipe 58.

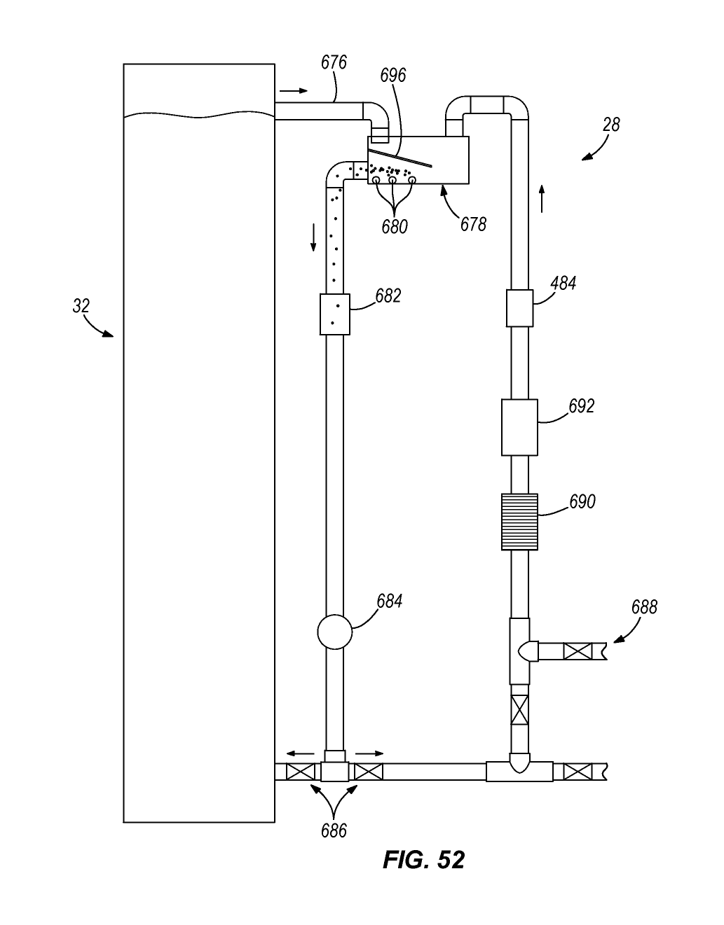

With reference to FIG. 52, a container 32 and a portion of an exemplary liquid management system 28 is shown. In the illustrated exemplary embodiment, the liquid management system 28 includes a water spillway pipe 676, a mixing tank 678, a gas injector or diffuser 680, a pH injector 682, a pump 684, a first set of valves 686, additional process plumbing 688, a filter 690, a sterilizer 692, and a pH sensor 484. The spillway pipe 676 is positioned near a top of the container 32 and receives water from the top of the container 32 that rises above the level of the spillway pipe 676. Water from the spillway pipe 676 is introduced into the mixing tank 678 and gas is introduced into the water present in the mixing tank 678 via the gas diffuser 680. A plate 696 is disposed in the mixing tank 678 above the gas diffuser 680 to assist with directing gas rising upward out of the water back toward the water and toward downstream pipes of the liquid management system 28. The introduced gas is generally referred to as a gas feed stream and may comprise about 12% of carbon dioxide by volume. Alternatively, the feed stream may comprise other percentages of carbon dioxide.

The pump 684 moves the combined water and bubbled gas through the pipes and creates a pressure differential in the pipes to facilitate said movement. Water pressure increases as the combined water and bubbled gas are pumped downward by the pump 684. This increased water pressure passes the bubbled gas into the water and transforms the gas bubbles into bicarbonate within the water. Algae have a much easier time absorbing carbon dioxide from bicarbonate in the water than from gas bubbles in the water. The water and bicarbonate mixture may now be pumped into the bottom of the container 32 or may be diverted for further processing. The first set of valves 686 is selectively controlled to divert the water and bicarbonate mixture as desired. In some instances, it may be desirable to pump all the water and bicarbonate mixture into the container 32. In other instances, it may be desirable to pump none of the water into the container and pump all of the water for further processing. In yet other instances, it may be desirable to pump some of the water and bicarbonate mixture into the container 32 and pump some of the mixture for further processing. In the event a constant volume of water is desired in the container 32, the amount of water spilling-off the top of the container 32 should equal the amount of water being pumped back into the bottom of the container 32.

The water and bicarbonate mixture pumped into the container 32 enters the container 32 near a bottom of the container 32 and mixes with the water already present in the container 32. This newly introduced mixture provides a new source of bicarbonate for the algae, thereby promoting cultivation of the algae within the container 32.

Water not diverted into the container 32 may be diverted downstream to a variety of additional processes. The additional process plumbing 688 of the liquid management system 28 is generically represented in FIG. 52 and may assume any configuration in order to accommodate a wide variety of water treatment processes. For example, the additional process plumbing 688 may divert the water through a water clarifier, a heat exchanger, solids removal equipment, ultra filtration and/or other membrane filtration, centrifuges, etc. Other processes and associated plumbing are possible and are within the intended spirit and scope of the present invention.

The water may also be diverted through a filter 690 such as, for example, a carbon filter for removing impurities and contaminants from the water. Exemplary impurities and contaminants may include invasive microbes that may have negative effects on algae growth such as bacterial and virus infection and predation. The liquid management system 28 may include a single filter or multiple filters and may include types of filters other than the exemplary carbon filter.

The water may further be diverted through a sterilizer 692 such as, for example, an ultraviolet sterilizer, which also removes impurities and contaminants from the water. The liquid management system 28 may include a single sterilizer or multiple sterilizers and may include types of sterilizers other than the exemplary ultraviolet sterilizer.

The water may additionally be diverted by a pH sensor 484 for determining the pH of the water. If the water has a higher than desired pH, the pH of the water is lowered to a desired level. Conversely, if the was has a lower than desired pH, the pH of the water is raised to a desired level. The pH of the water may be adjusted in a variety of different manners. Only some of the many manners for adjusting the pH of the water will be described herein. The description of these exemplary manners of adjusting the pH is not intended to be limiting. In a first example, the pH injector 682 is used to adjust the pH of the water. In this example, the pH injector 682 is disposed in the pipe between the mixing tank 678 and the pump 684. Alternatively, the pH injector 682 may be disposed in other locations in the liquid management system 28. The pH injector 682 injects an appropriate type and quantity of substance into the water stream passing through the pipe to change the pH of the water to the desired level. In another example, the gas diffuser 680 may be used to adjust the pH level of the water. The quantity of carbon dioxide present in water determines the pH of the water. Generally, the more carbon dioxide present in water, the lower the pH level of the water. Thus, the quantity of carbon dioxide introduced into the water via the gas diffuser 680 may be controlled to raise or lower the pH level of the water as desired. More particularly, when the pH sensor 484 takes a pH reading and it is determined that the pH level of the water is higher than desired, the gas diffuser 680 may increase the rate at which carbon dioxide is introduced into the water. Conversely, when the pH level of the water is lower than desired, the gas diffuser 680 may decrease the rate at which carbon dioxide is introduced into the water. In a further example, the pH injector 682 may be used to inject carbon dioxide into the water in addition to the carbon dioxide introduced by the gas diffuser 680. In this way, the pH injector 682 is adjustable to control the amount of additional carbon dioxide introduced into the water to maintain a desired pH level.

After the water is diverted through water treatment processes such as those described herein, the water is pumped back into the mixing tank 678 where the water is mixed with new water introduced into the mixing tank 678 from the spillway pipe 676. The water then flows downstream as described above. Alternatively, the water may be diverted directly into the container 32 rather than into the mixing tank 678.

It should be understood that the water treatment processes used for removing impurities and contaminants from the water both decrease the adverse effects that such impurities and contaminants have on algae cultivation and improve water clarity. Improved water clarity allows light to better penetrate the water, thereby increasing the algae's exposure to light and improving algae cultivation.

It should also be understood that the container's ability to support the algae on the media 110 during the cultivation process and maintain a low concentration of algae in the water, increases the effectiveness of the water treatment processes described above and illustrated in FIG. 52. More particularly, moving water with a low concentration of algae therein through the components of the liquid management system 28 illustrated in FIG. 52 inhibits fouling and clogging of the components with algae. In other words, very little algae are present in the water to foul or clog the pipes, gas diffuser, pump, filter, etc. In addition, a low concentration of algae in the water inhibits the filter and sterilizer from removing or killing a large quantity of algae, which would ultimately adversely affect algae cultivation. In some exemplary embodiments, the concentration of algae supported on the media versus the concentration of algae suspended in the water is 26:1. In other exemplary embodiments, the concentration of algae supported on the media versus the concentration of algae suspended in the water may be 10,000:1. The system 20 is capable of providing lower and higher algae concentration ratios than the exemplary ratios disclosed herein and are within the intended spirit and scope of the present invention.

With reference to FIG. 53, an exemplary support structure 396 is illustrated for supporting a container 32 in a vertical manner. This exemplary support structure 396 is for illustrative purposes and is not intended to be limiting. Other support structures for supporting a container 32 in a vertical manner are contemplated and are within the spirit and scope of the present invention. In the illustrated exemplary embodiment, the support structure 396 includes a base 400 supportable on a ground or floor surface, an upright member 404 extending upward from the base 400, and a plurality of couplings 408 supported by the upright member 404 and extending from the upright member 404 at different heights to engage the container 32. The base 400 supports both the container 32 and the upright member 404 from below. The upright member 404 includes a pair of vertical beams 412 and a plurality of cross beams 416 extending between the vertical beams 412 to provide support, strength, and stability to the vertical beams 412. In the illustrated exemplary embodiment, the support structure 396 includes four couplings 408, each coupling 408 comprising a band 420 extending around the container housing 76 and a bushing 424 disposed between the band 420 and the container housing 76. The base 400 provides the substantial amount of vertical support for the container 32, while the upright member 404 and the couplings 408 provide the substantial amount of horizontal support for the container 32.

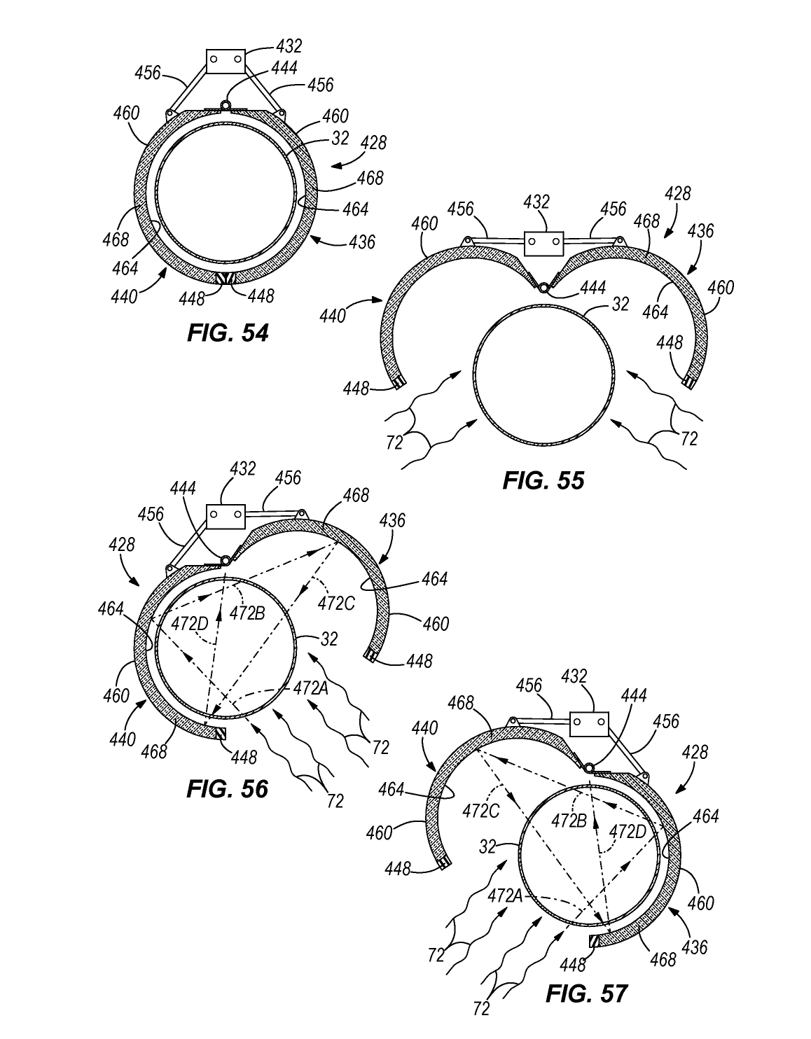

With continued reference to FIG. 53 and additional reference to FIGS. 54-58, an environmental control device (ECD) 428 is illustrated and assists with maintaining a desirable environment for cultivating algae within the container 32. The illustrated ECD 428 is for illustrative purposes and is not intended to be limiting. Other shapes, sizes, and configurations of the ECD 428 are contemplated and are within the intended spirit and scope of the present invention.

With particular reference to FIGS. 53 and 54, the illustrated exemplary ECD 428 has a “clam-shell” type shape. More particularly, the ECD 428 includes first and second semi-circular members 436, 440, a hinge or other pivotal joint 444 connected to first adjacent ends of the first and second semi-circular members 436, 440, and a sealing member 448 connected to each of second adjacent ends of the first and second semi-circular members 436, 440. The hinge 444 allows the first and second members 436, 440 to pivot relative to each other about the hinge 444 and the sealing members 448 abut each other when the first and second members 436, 440 are both fully closed to provide a seal between the first and second members 436, 440.

With reference to FIG. 53, the ECD 428 includes three sets of first and second members 436, 440, one set between each of the couplings 408. In the illustrated exemplary embodiment, the ECD 428 comprises three sets of first and second members 436, 440 to accommodate the use of four couplings 408. As indicated above, the support structure 396 may include any number of couplings 408 and, accordingly, the ECD 428 may include any number of sets of first and second members 436, 440 having any length to accommodate the space between the number of couplings 408. For example, the support structure 396 may include only two couplings 408, the bottom coupling 408 and the top coupling 408, and the ECD 428 may only require one tall set of first and second members 436, 440 to surround the container 32 along substantially its entire height between the top and bottom couplings 408.

With continued reference to FIGS. 53 and 54, the ECD 428 includes a motor 432 for opening and closing the first and second members 436, 440, a drive shaft 452 coupled to the motor 432, and a plurality of linkage arms 456 coupled to the drive shaft 452 and an associated one of the first and second members 436, 440. Activation of the motor 432 drives the drive shaft 452, which applies a force on the linkage arms 456 to either open or close the first and second members 436, 440. The motor 432 is coupled to and controllable by the controller 40. In the illustrated exemplary embodiment, a single motor 432 is used to open and close all of the sets of first and second members 436, 440. Alternatively, the ECD 428 may include one motor 432 per set of first and second members 436, 440 to independently open and close sets of the first and second members 436, 440, or one motor 432 for each first member 436 and one motor 432 for each second member 440 to drive the first and second members 436, 440 independently of each other, or any number of motors 432 to drive any number of first and second members 436, 440 or sets of first and second members 436, 440. With each motor 432 included, a separate drive shaft 452 will be associated with each motor 432 to output the driving force of each motor 432. Alternatively, each motor 432 may include multiple drive shafts 452. For example, a motor 432 may include two drive shafts 452, a first drive shaft 452 for opening and closing a first member 436 and a second drive shaft 452 for opening and closing a second member 440.

Referring now to FIGS. 54-57, the first and second members 436, 440 are movable to a variety of different positions and may both be moved together or may be moved independently of each other. The first and second members 436, 440 may be positioned in a fully closed position (see FIG. 54), a fully opened position (see FIG. 55), a half-opened position with the first member 436 fully opened and the second member 440 fully closed (see FIG. 56), another half-opened position with the second member 440 fully opened and the first member 436 fully closed (see FIG. 57), or any of a variety of other positions between the fully opened and the fully closed positions.

With continued reference to FIGS. 54-57, each of the first and second members 436, 440 includes an outer surface 460, an inner surface 464, and a core 468 between the outer and inner surfaces 460, 464. The outer surface 460 may be made of a variety of materials such as, for example, stainless steel, aluminum, fiber reinforced plastic (FRP), polypropylene, PVC, polyethylene, polycarbonate, carbon fiber, etc. The outer surface 460 may be white or light colored and may be capable of reflecting light. The outer surface 460 may also be smooth to resist dirt or other debris from attaching thereto. The core 468 may be made of a variety of materials such as, for example, blanket of closed neoprene, encapsulated insulation, formed insulation material, molded foam, etc. The core 468 preferably has the characteristics to insulate the container from both hot and cold conditions as desired. The inner surface 464 may be made of a variety of materials such as, for example, stainless steel, aluminum, fiber reinforced plastic (FRP), polypropylene, PVC, polyethylene, polycarbonate, carbon fiber, etc. In some embodiments, the outer and inner surfaces 460, 464 may be made of the same material and share the same characteristics. The inner surface 464 preferably has reflective characteristics in order to reflect light rays in a desired manner (describe in greater detail below). To provide such reflective characteristics, the inner surface 464 may be made of a reflective material or may be coated with a reflective substance. For example, the inner surface 464 may include a thin layer of mirror material, MYLAR®, glass bead impregnated, embedded silvered aluminum plate, a reflective paint, etc.

As indicated above, the ECD 428 is capable of assisting with controlling the environment for cultivating algae within the container 32. More particularly, the ECD 428 is capable of affecting the temperature within the container 32 and affecting the amount of sunlight contacting the container 32.

Regarding affecting temperature, the ECD 428 has the capability to selectively insulate the container 32. With the first and second members 436, 440 in the fully closed position (see FIGS. 53 and 54), the container 32 is surrounded by the first and second members 436, 440 along a substantial portion of its height. When the ambient temperature outside is below a desired temperature within the container 32, the first and second members 436, 440 may be moved to their fully closed position to insulate the container 32 and assist with keeping the colder ambient air from cooling the temperature within the container 32. When the ambient temperature outside is above a desired temperature within the container 32, the first and second members 436, 440 may again be moved to their fully closed position to reflect the intense sunlight rays and prevent the sunlight rays from contacting the container 32. Alternatively, when the ambient temperature outside is above a desired temperature within the container 32, the first and second members 436, 440 may be moved to their fully opened position (see FIG. 55) to move the insulated first and second members 436, 440 away from the container 32 and allow cooling of the container 32 (e.g., cool by convection). The first and second members 436, 440 may be moved to any desired positions to assist with maintaining the temperature within the container 32 at a desired temperature.

Regarding affecting the amount of sunlight contacting the container 32, the first and second members 436, 440 may be moved to any desired position to allow a desired amount of sunlight to contact the container 32. The first and second members 436, 440 may be moved to their fully closed position to prevent sunlight 72 from contacting the container 32 (see FIG. 54), the first and second members 436, 440 may be moved to their fully opened positions so as not to interfere with the amount of sunlight 72 contacting the container 32 (i.e., allowing the full amount of sunlight to contact the container—see FIG. 55), or the first and second members 436, 440 may be moved to any positions between the fully closed and fully opened positions to allow a desired amount of sunlight to contact the container 32 (see FIGS. 56 and 57).

As indicated above, the inner surface 464 of the ECD 428 is made of a reflective material capable of reflecting sunlight 72. The reflective capabilities of the inner surface 464 may improve the efficiency at which the sunlight 72 contacts the container 32. More particularly, sunlight 72 emitted toward the container 32 may: contact the container 32 and algae therein; pass through the container 32 without contacting the algae; or miss the container 32 and algae altogether. For the latter two scenarios, the ECD 428 may assist with reflecting the sunlight not contacting the algae into contact with the algae.

With reference to FIGS. 56 and 57, two exemplary reflective paths 472 along which sunlight 72 may be reflected back into contact with the algae are illustrated. These illustrated exemplary reflective paths 472 are only two paths of many paths along which sunlight 72 may be reflected by the inner surface 464 of the ECD 428. These reflective paths 472 are shown for illustrative purposes and are not intended to be limiting. Many other reflective paths 472 are possible and are within the intended spirit and scope of the present invention. With reference to the illustrated exemplary reflective paths 472, sunlight 72 may pass through the containers 32 without contacting algae within the containers 32 as represented by first portions 472A of the paths and contact the inner surfaces 464 of the first and second members 436, 440 of the ECD 428. The inner surfaces 464 reflect the sunlight 72 in a second direction as represented by second portions 472B of the paths. As can be seen, the second portions 472B of the paths pass through the containers 32. Some of this sunlight 72 will contact algae within the containers 32, while some of the sunlight 72 will again pass through the containers 32 without contacting the algae. This sunlight 72 passing through the containers 32 will engage the inner surfaces 464 of the other members 436, 440 and reflect back towards the containers 32 as represented by third portions 472C of the paths. The reflected sunlight 72 again passes through the containers 32 and some of the sunlight 72 contacts algae within the containers 32, while some of the sunlight 72 again passes through the containers 32 without contacting algae. This sunlight 72 passing through the containers 32 engages the inner surfaces 464 of the members 436, 440 originally engaged by the sunlight 72 and reflects again through the containers 32 as represented by fourth portions 472D of the paths. Some of this sunlight 72 contacts algae within the containers 32, while some of the sunlight 72 still passes through without contacting algae. Sunlight reflection may continue until the sunlight 72 contacts the algae or until the sunlight 72 is reflected away from the containers 32 and the inner surfaces 464 of the first and second members 436, 440. As can be seen, the reflective inner surfaces 464 of the first and second members 436, 440 provide additional opportunities for sunlight 72 to contact the algae within the container 32 and promote photosynthesis. Without the reflective capabilities of the ECD 428, sunlight 72 passing through or passing by the containers 32 would not have another opportunity to contact the algae within the container 32.

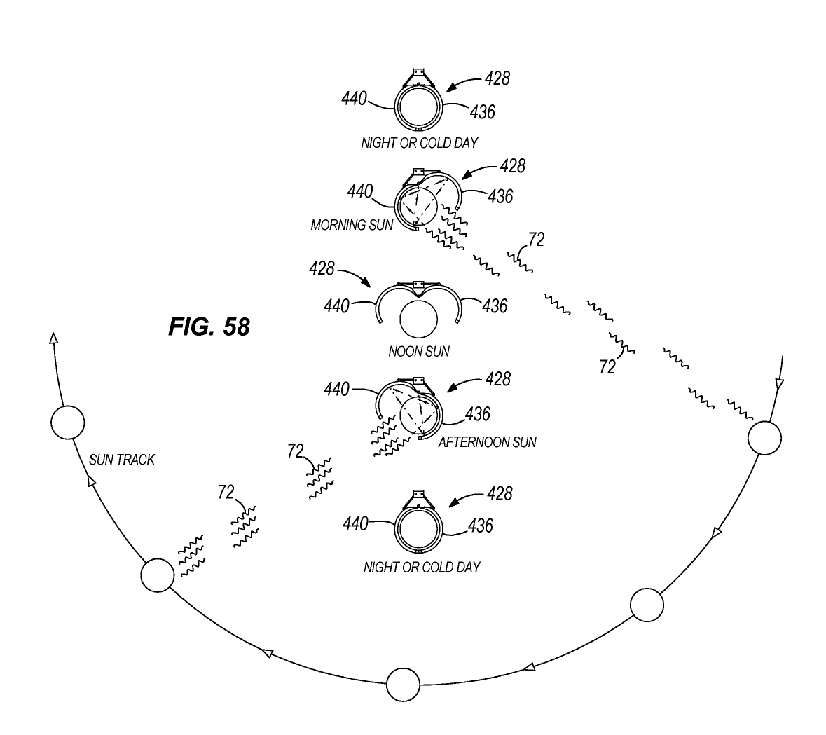

Referring now to FIG. 58, the ECD 428 may be utilized to optimize the temperature within the container 32 and optimize the amount of sunlight 72 contacting the container 32 and the algae throughout the day. The figures of the ECD 428 represent exemplary positions occupied by the ECD 428 during different times of the day. FIG. 58 also illustrates a schematic representation of a path of the sun throughout a single day. The orientations of the ECD 428 illustrated in FIG. 58 are for illustrative purposes and are not intended to be limiting. The orientations of the ECD 428 illustrated in FIG. 58 exemplary a portion of the many orientations the ECD 428 is capable of occupying. Many other orientations are contemplated and are within the spirit and scope of the present invention.

The top figure of the ECD 428 shows the ECD 428 in an exemplary orientation that may be occupied during nighttime or during a cold day in order to insulate the container 32 and maintain a desirable temperature within the container 32. The second figure from the top shows the ECD 428 in an exemplary orientation that may be occupied during the morning. In the morning, the sun is generally positioned to one side of the container 32 and it may be desirable to have one of the members to the side of the sun opened (first member 436 as illustrated) to allow sunlight 72 to contact the container 32 and keep the other member to the opposite side of the sun closed (second member 440 as illustrated) in order to provide the reflective capabilities described above. The third figure from the top shows the ECD 428 in an exemplary orientation that may be occupied during noon or the middle of the day. During the middle of the day, the sun is usually high in the sky and directly over (or in front of as illustrated in FIG. 58) the container 32. With the sun in such a position, it may be desirable to have both the first and second members 436, 440 open to allow the greatest amount of sunlight 72 to contact the container 32. The first and second members 436, 440 may also provide reflective capabilities as described above for reflecting sunlight 72 toward the container 32. The fourth figure from the top shows the ECD 428 in an exemplary orientation that may be occupied during the afternoon. In the afternoon, the sun is generally positioned to one side of the container 32 (opposite the morning sun) and it may be desirable to have one of the members to the side of the sun opened (second member 440 as illustrated) to allow sunlight 72 to contact the container 32 and keep the other member to the opposite side of the sun closed (first member 436 as illustrated) in order to provide the reflective capabilities described above. The bottom figure shows the ECD 428 again in an exemplary orientation occupied during nighttime or on cold days. As indicated above, the orientations of the ECD 428 illustrated in FIG. 58 are only exemplary orientations that may be occupied during a day. The ECD 428 may occupy different orientations during various times throughout a day for various reasons such as, for example, the environmental conditions surrounding the container 32, the type of algae within the container 32, the desired performance of the container 32, etc.

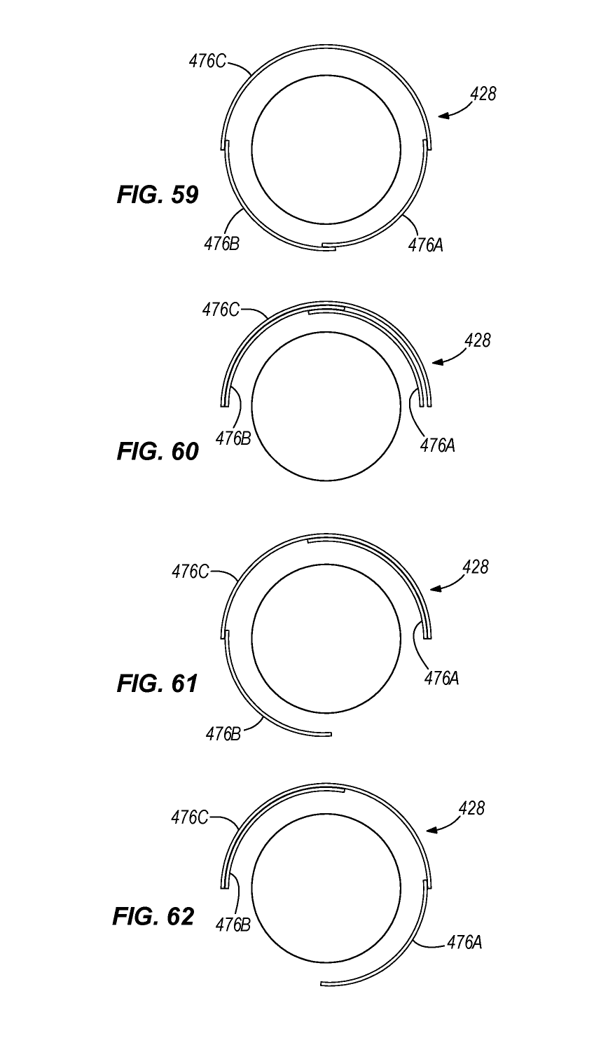

It should be understood that the ECD 428 is capable of having configurations other than the illustrated exemplary clam-shell configuration. For example, the ECD 428 may include a plurality of semi-circular members 476 that together concentrically surround the container 32 and are slidable around the container 32 such that the members 476 overlap or nest within each other when moved to their open positions (see FIGS. 59-62). In the illustrated example, the first and second members 476A, 476B move relative to each other and the container 32 to expose the container 32 as desired. A third member 476C is disposed behind the container 32, typically on a side of the container 32 opposite the position of the sun, and may be stationary or movable.

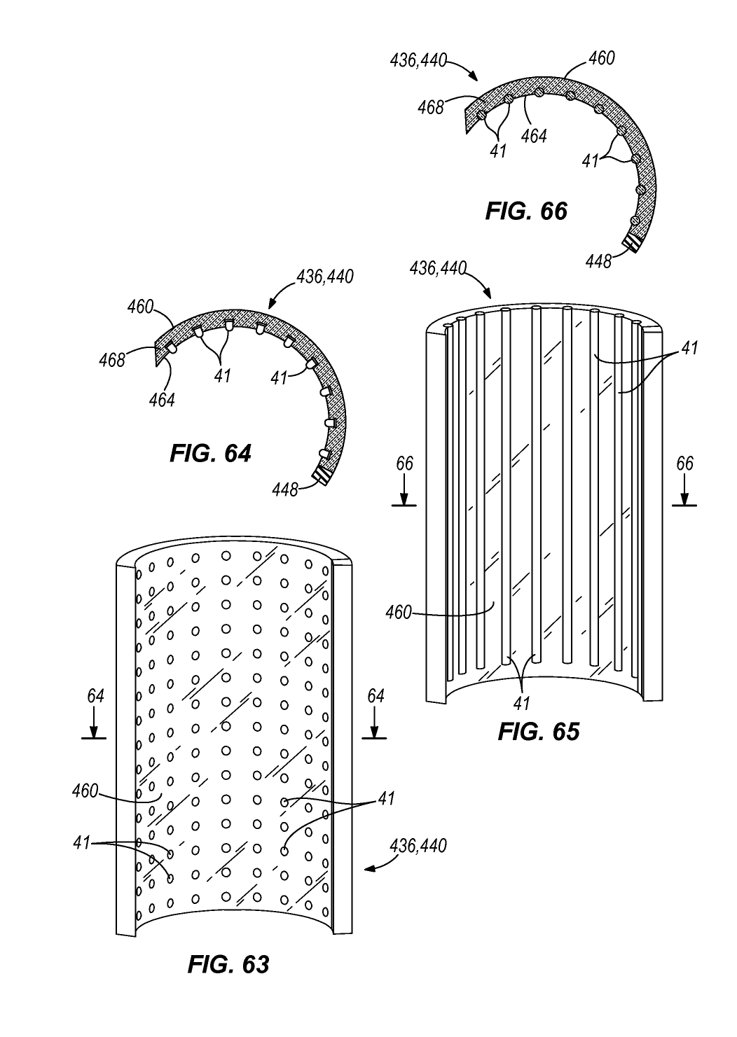

Referring now to FIGS. 63 and 64, the ECD 428 may include an artificial light system 37. Components similar between the previously described and illustrated container, artificial light systems, and ECD, and the container, artificial light systems, and ECD illustrated in FIGS. 63 and 64 are identified by the same reference numbers.

In the illustrated exemplary embodiment, the artificial light system 37 includes a light source 41 comprised of an array of LEDs coupled to the inner surface 464 of the first and second members 436, 440 (only one member shown). The LEDs 41 are electrically connected to an electrical power source and to the controller 40. The LEDs 41 operate and may be controlled in same manner as the other artificial light systems 37 described herein to emit light onto the container 32 and the algae. In some embodiments, the LEDs 41 may be imbedded in the inner surface 464 such that the LEDs 41 are flush with the interior surface 464. In such embodiments, the inner surface 464 may be stamped with perforations that match the desired LED array formation to receive the LEDs 41 and position the LEDs flush with the inner surface 464.

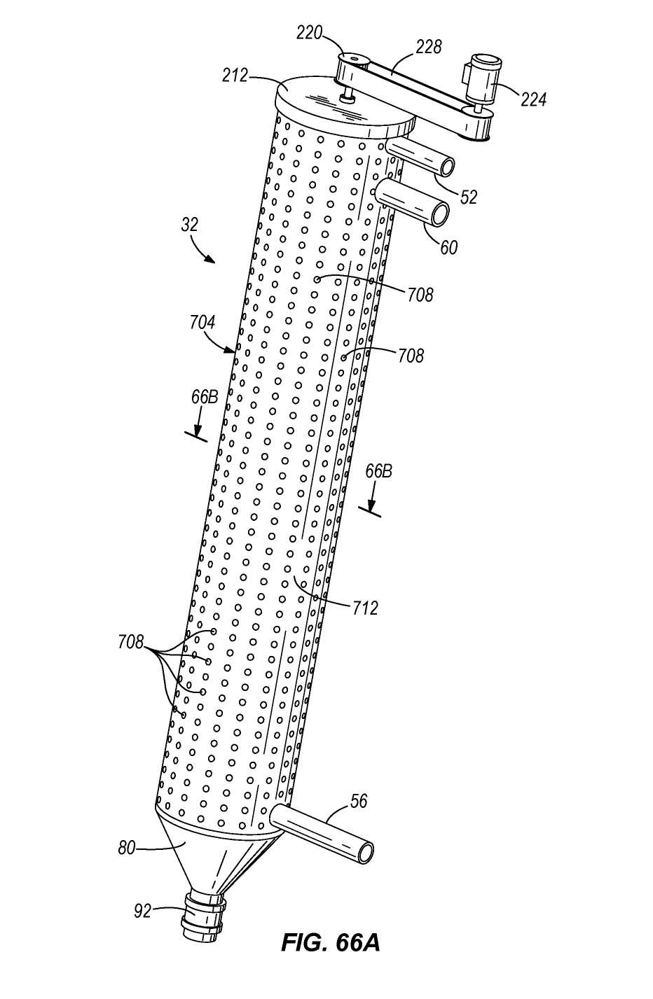

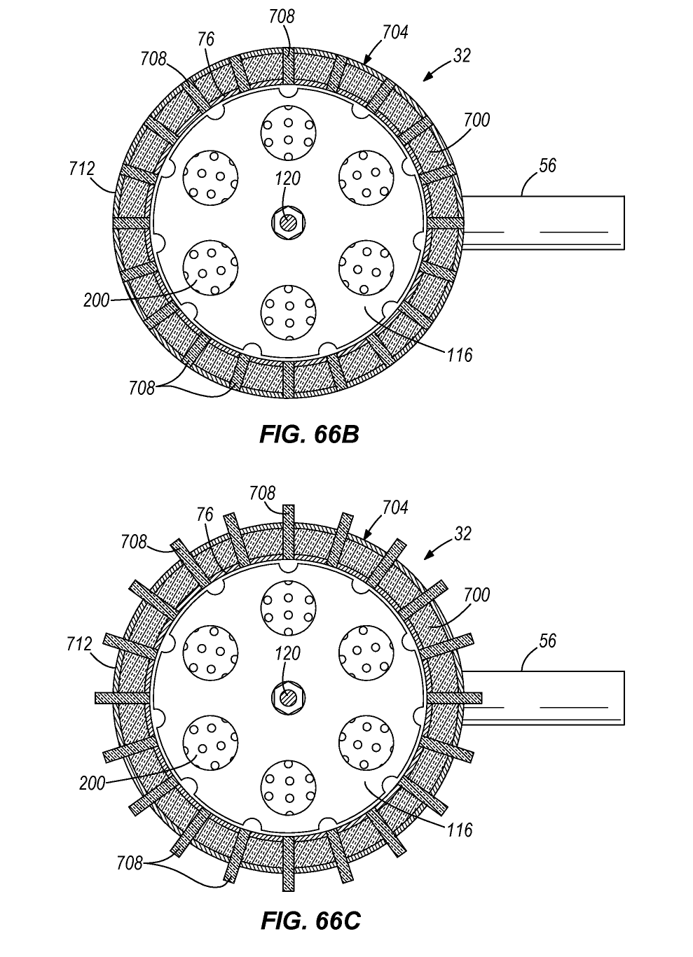

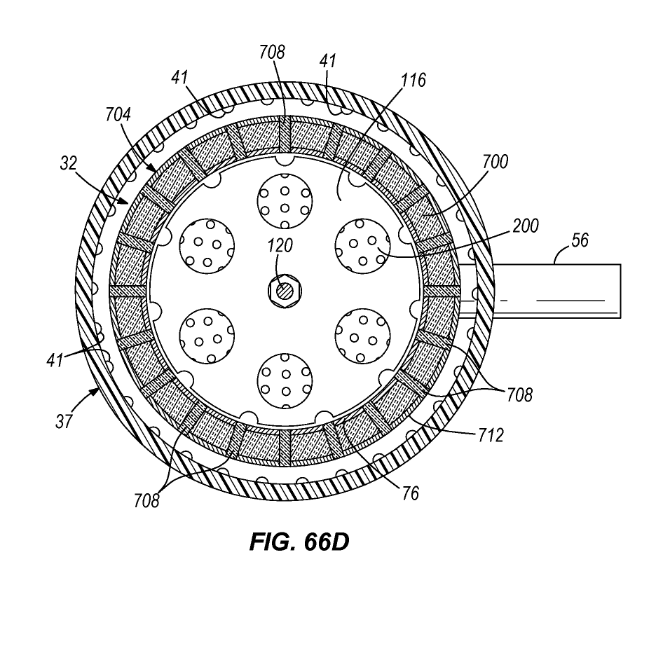

Referring to FIGS. 65 and 66, the ECD 428 includes an alternative embodiment of an artificial light system 37. Components similar between the previously described and illustrated container, artificial light systems, and ECD, and the container, artificial light systems, and ECD illustrated in FIGS. 65 and 66 are identified by the same reference numbers.

In this illustrated exemplary embodiment, the artificial light system 37 includes a light source 41 comprised of a plurality of fiber optic light channels imbedded in the inner surface 464 of the first and second members 436, 440 (only one member shown). The fiber optic light channels 41 may receive light in a variety of manners including LEDs or other light emitting devices or from a solar light collection apparatus oriented to receive sunlight 72 and transfer the collected sunlight 72 to the light channels 41 via fiber optic cables. The light channels 41 may be controlled by the controller 40 as desired.

Referring now to FIGS. 66A and 66B, another exemplary embodiment of a container 32 is illustrated. In this illustrated exemplary embodiment, the housing 76 is made of an opaque material that does not allow a substantial quantity of light to penetrate the housing 76. The housing 76 may be made of a variety of different materials such as, for example, metal, opaque plastics, concrete, fiberglass, lined structures, etc. The container 32 also includes an insulation layer 700 surrounding the housing 76 for thermally insulating the container 32 and an outer layer 704 positioned externally of and surrounding the insulation layer 700 for protecting the insulation layer 700. The insulation layer 700 may be comprised of a variety of different materials such as, for example, plastic, fiberglass, rock wool, closed and open celled polystyrene, polyurethane foam, cellulose fiber, etc., and the outer layer 704 may be comprised of a variety of different materials such as, for example, plastic, fiberglass, metal, paint, sealing agents, etc. It should be understood that in some exemplary embodiments where at least one of the insulation layer 700 and the outer layer 704 is comprised of an opaque material, the housing 76 of the container 32 may be translucent or transparent.

With continued reference to FIGS. 66A and 66B, the container 32 further includes a plurality of light elements 708 for transmitting light from the exterior of the container 32 to an interior of the container 32 for purposes of cultivating algae therein. In some exemplary embodiments, the material that comprises the light elements 708 may include an infrared inhibitor or infrared filter applied to the light elements 708 or included in the composition of the light element material in order to reduce or limit the heat build-up that occurs in the light elements 708 as light passes therethrough. In the illustrated exemplary embodiment, the light elements 708 are positioned in holes defined through the housing 76, the insulation layer 700, and the outer layer 704. Each light element 708 is flush at its ends with the interior surface 196 of the housing 76 and an outer surface 712 of the outer layer 704. The light elements 708 are sealed within the holes in an air and water tight fashion to prevent water within the container 32 from leaking into the holes. The light elements 708 may be made of a variety of light transmitting materials such as, for example, glass fiber, fiber optic, plastics such as acrylic, etc., in order to receive light externally of the container 32 and transmit the collected light toward the interior of the container 32 for purposes of cultivating algae within the container 32. Also, the light elements 708 may be made of materials that do not degrade or are otherwise adversely affected by exposure to light or to liquids disposed within or outside of the container 32. In the illustrated exemplary embodiment, the light elements 708 are adapted to receive natural light from the Sun. Also, in the illustrated exemplary embodiment, the end of the light elements 708 adjacent the outer layer 704 (i.e., the exterior end) is flush with the outer surface 712 of the outer layer 704.

With reference to FIG. 66C, the exterior end of the light elements 708 may extend beyond the outer surface 712 of the outer layer 704. In such embodiments, the exterior end of the light elements 708 may be angled toward the Sun in order to optimally align the exterior end with the Sun.

With containers 32 constructed in the manner described above and illustrated in FIGS. 66A-66C, the containers 32 may be made of materials that are less expensive, more durable, and more resistant to thermal and environmental conditions. These containers 32 may eliminate a desire to have a secondary structure surrounding the containers 32 to provide protection from thermal and environmental conditions. Incorporation of the light elements 708 facilitates light transmission into the containers 32 when the containers 32 are constructed in the manner described with reference to FIGS. 66A-66C.

Referring now to FIG. 66D, another alternative exemplary embodiment of a container 32 is illustrated. The container 32 illustrated in FIG. 66D has many similar elements to the containers 32 illustrated in FIGS. 66A-66C and such similar elements are identified by similar reference numbers. In FIG. 66D, an artificial light system 37 is disposed externally of and emits light toward the container 32. In the illustrated exemplary embodiment, the artificial light system 37 completely surrounds a periphery of the container 32. In other exemplary embodiments, the artificial light system 37 may not completely surround a periphery of the container 32. In yet other exemplary embodiments, a plurality of artificial light systems 37 may be disposed at various locations around the container 32. No matter the embodiment, the artificial light system 37 is used to provide light to the light elements 708, which receive the light and transmit the light toward an interior of the container 32. The artificial light system 37 may be the sole source of light provided to the container 32 or the artificial light system 37 may be used in conjunction with natural sunlight to satisfy the lighting needs of the container 32.

Now that the structure of the algae cultivation system 20 has been described, operation of the system 20 will be described herein. The following description relating to operation of the algae cultivation system 20 only exemplifies a sample of the variety of possible manners for operating the system 20. The following description is not intended to be limiting upon the algae cultivation system 20 and the manners of operation.

Referring back to FIGS. 1 and 2, carbon dioxide is harvested from one or more of a variety of different carbon dioxide sources 44. Harvesting carbon dioxide from emissions generated as a byproduct of a manufacturing or industrial process is particularly helpful for the environment by reducing the amount of carbon dioxide exhausted into the environment. Carbon dioxide can also be provided by a variety of different sources 44 not shown, but represented generically by the Nth block. The resulting carbon dioxide is delivered from the carbon dioxide source or sources 44 to the containers 32 via gas processing components such as, for example, carbon dioxide cooling systems, and toxic gas and compound scrubbing systems, and a network of pipes 48 of the gas management system 24. Before the carbon dioxide is delivered to the containers 32, the containers 32 should be filled with a sufficient level of water and an initial amount of algae (otherwise known as seeding algae). The water is provided to the containers 32 via water inlet pipes 56 of the liquid management system 28 and the algae can be introduced into the containers 32 in a variety of manners. If the containers 32 are “virgin” containers (i.e., no previous algae cultivation has occurred in the containers or the containers have been cleaned to completely remove the presence of algae), algae can be introduced into the liquid management system 28 and delivered to the containers 32 with the water supply. Alternatively, if the containers 32 have previously been used for algae cultivation, algae may already be present in the containers 32 from the prior cultivation process. In such instances, only water needs to be supplied to the containers 32. After the containers 32 are sufficiently supplied with water and algae, carbon dioxide is supplied to the containers 32 via the gas management system 24. As illustrated in FIGS. 1 and 2, the gas and liquid management systems 24, 28 are electronically coupled to and controlled by the controller 40.

The media 110 utilized in the algae cultivation system 20 facilitates productive algae cultivation for a variety of reasons. First, the media 110 is comprised of a material that is suitable for algae growth. In other words, the media 110 is not composed of a material that hinders growth of or kills the algae. Second, the media 110 consists of a material to which the algae can attach and upon which the algae can rest during its growth. Third, the media 110 provides a large quantity of dense surface area on which the algae can grow. The large quantity of available media surface area entices the algae to grow on the media 110 rather than be suspended in the water, thereby contributing to a large quantity of the algae being supported on the media 110 and only a small quantity of algae remaining suspended in the water. In other words, a higher concentration of the total quantity of algae present in the container 32 is supported on the media 110 than is suspended in the water. The small quantity of algae suspended in the water does not significantly inhibit penetration of sunlight 72 into the housing 76, thereby improving the efficiency of photosynthesis taking place within the container 32. Fourth, the large quantity of media 110 within the cavity 84 of the housing 76 acts to inhibit and slow ascent of the carbon dioxide to the top of the housing 76, thereby increasing the amount of time the carbon dioxide resides in the water proximate the algae supported on the media 110. Increasing the time carbon dioxide resides proximate the algae, increases the absorption of the carbon dioxide by the algae and increases the growth rate of the algae. Fifth, the media 110 provides protection to the algae supported thereon just before and during extraction of the algae and water from the containers 32 (described in greater detail below). While a variety of benefits of the media 110 are described herein, this list is not exhaustive and is not meant to be limiting. The media 110 may provide other benefits to algae cultivation.

With continued reference to FIGS. 1 and 2 and additional reference to FIG. 3, the frames 108 are rotatable within the containers 32 relative to their respective housings 76. In the illustrated exemplary embodiment, a single motor 224 is coupled to multiple frames 108 to rotate the multiple frames 108 relative to their respective housings 76. Alternatively, a separate motor 224 can be used to drive each frame 108 or any number of motors 224 can be utilized to drive any number of frames 108. No matter the number of motors 224 or the manner in which the motor(s) 224 drive the frames 108, the motor(s) 224 is (are) all electronically coupled to the controller 40 and controllable by the controller 40 to activate and deactivate the motor(s) 224 accordingly. In the following description, only a single motor 224 will be referenced. As indicated above, the motor 224 is part of the drive mechanism, which also includes a belt or chain 228 coupled between the motor 224 and the gears 220 connected to ends of the shafts 120. When rotation of the frames 108 is desired, the controller 40 activates the motor 224 to drive the belt 228, gears 220, and shafts 120, thereby rotating the frames 108 and the media 110 attached to the frames 108 relative to the housings 76. In some exemplary embodiments, the frames 108 may be rotated in a single direction. In other exemplary embodiments, the frames 108 may be rotated in both directions.

Rotation of the frames 108 and media 110 is desirable for several reasons. First, the frames 108 and media 110 are rotated to expose the algae supported on the media 110 to the sunlight 72 and/or the artificial lighting system 37 as desired. Rotation of the frames 108 in this manner exposes all of the media 110 and all of the algae to the light 37, 72 in a substantially proportional manner or in a manner that is most efficient for algae cultivation. In addition, rotation of the frames 108 in this manner also moves the media 110 and algae out of the light 37, 72 and into a shaded or dark portion of the containers 32, thereby providing the dark phase necessary to facilitate the photosynthesis process. The frames 108 and media 110 can be rotated in a variety of methods and speeds. In some embodiments, rotation of the frames 108 can be incremental such that rotation is started and stopped at desired increments of time and desired increments of distance. In other embodiments, the frames 108 rotate in a continuous uninterrupted manner such that the frames 108 are always rotating during the algae cultivation process. Thus, the outermost strands of media 110 continuously wipe the interior surfaces 196 of the housings 76. In either of the embodiments described above, the rotation of the frames 108 is relatively slow such that the algae supported on the media 110 is not dislodged from the media 110.

Rotation of the frames 108, as discussed above, also provides another benefit to the algae cultivation system 20. The outer most strands of media 110 extending between the recesses 132 defined in the upper and lower connector plates 112, 116 contact the interior surface 196 of the housings 76. As the frames 108 rotate, the outermost media strands 110 wipe against the interior surfaces 196 of the housings 76 and clear the algae attached to the interior surfaces 196. Algae attached to the interior surfaces 196 of the housings 76 significantly reduce the amount of light 37, 72 penetrating the housings 76 and entering the cavities 84, thereby negatively affecting photosynthesis and algae growth. Accordingly, this wiping of the interior surfaces 196 improves light 37, 72 penetration through the housings 76 and into the cavities 84 to maintain desired levels of algae cultivation. For example, during algae cultivation, the frames 108 may be rotated at a rate in a range between about one 360° rotation every few hours to about one 360° rotation in less than one minute. These exemplary rotations are for illustrative purposes and are not intended to be limiting. The frames 108 are capable of being rotated at a variety of other rates, which are still within the spirit and scope of the present invention.

Rotation of the frames 108, as discussed above, provides yet another benefit to the algae cultivation system 20. Rotation of the frames 108 cause oxygen bubbles within the water and stuck to the media 110 or algae to dislodge and ascend toward the top of the containers 32. The oxygen may then be exhausted from the containers 32 via the gas discharge pipes 52. High oxygen levels within the containers 32 may inhibit the photosynthesis process of the algae, thereby decreases productivity of the system 20. Rotation of the frames 108 in the first manner described above may be sufficient to dislodge the oxygen from the media 110 and algae. Alternatively, the frames 108 may be jogged quickly, step rotated, or rotated quickly to remove the oxygen.

The oxygen exhausted via the gas discharge pipes 52 may be collected for resale or use in other applications. It is desirable for the collected oxygen to have a high oxygen level and a low level of other components such as, for example, carbon dioxide, nitrogen, etc. In some embodiments, the system 20 may be controlled to optimize the oxygen level and minimize the level of other components. One example of such embodiments for optimizing oxygen levels includes: shutting down the introduction of carbon dioxide into the containers 32, allowing an appropriate amount of time to pass, rotating the frames 108 in a desired manner to dislodge the oxygen after the appropriate amount of time has passed, opening the gas discharge pipes 52 (or other discharge valve/pipe/etc.), exhausting the oxygen through the gas discharge pipes 52, routing the exhausted oxygen to a storage vessel or downstream for further processing. In such an example, the system 20 may include a valve or solenoid in communication with the component(s) introducing the carbon dioxide in order to selectively control introduction of the carbon dioxide, a valve or solenoid in communication with the gas discharge pipes 52 in order to selectively control exhaustion of the oxygen from the containers 32, and a blower or other movement device for moving the exhausted oxygen from the containers 32 to either or both of the storage vessel and downstream for further processing. The algae cultivation cycle continues by closing the gas discharge pipes 52 and reintroducing carbon dioxide into the containers 32.

The frames 108 are also rotatable in a second manner for another purpose. More specifically, the frames 108 are rotated just before removal of the water and algae from the containers 32 in order to dislodge the algae from the media 110. Removal of the algae from the media 110 is desirable so that the algae can be removed from the containers 32 and harvested for fuel production. This rotation of the frames 108 is relatively fast in order to create sufficient centrifugal force to dislodge the algae from the media 110, but not too fast where the algae may be damaged. An exemplary rate at which the frames 108 and media 110 are rotated in this manner is about one rotation per second. Alternatively, the frames 108 and media 110 could be rotated at other speeds as long as the algae is dislodged from the media 110 in a desirable manner. Rotational rates of the frame 108 and media 110 may be dependent upon the type of algae species growing within the container 32. For example, the frame 108 and media 110 may rotate at a first speed for a first species of algae and may rotate at a second speed for a second species of algae. Different rotational rates may be necessary to dislodge the algae from the media 110 due to the characteristics of the algae species. Some algae species may stick or adhere to the media 110 to a greater extent than other algae species. In some embodiments, the rotation of the frames 108 is controlled to dislodge a majority of the algae from the media 110, but maintain a small amount of algae on the media 110 to act as seeding algae for the next cultivation process. In such embodiments, the introduction of algae into the containers 32 prior to initiating the next cultivation process is not required. In other embodiments, the rotation of the frames 108 is controlled to dislodge all of the algae from the media 110. In such embodiments, algae must be introduced into the containers 32 prior to initiating the next cultivation process. Algae may be introduced into the containers 32 with water via the liquid management system 28.

As indicated above, it is desirable to dislodge the algae from the media 110 prior to removing the water and algae combination from the containers 32. To do so, the controller 40 initiates the motor 224 to rotate the frames 108 at the relatively fast speed. This fast rotation also wipes the outermost media strands 110 against the interior surfaces 196 of the housings 76 to clear off any algae that may have accumulated on the interior surfaces 196 of the housings 76. With a substantial amount of the algae now disposed in the water, the water and algae combination may be removed from the containers 32. The controller 40 communicates with the liquid management system 28 to initiate removal of the water and algae from the containers 32 through the water outlets 100. A pump of the liquid management system 28 directs the water and algae combination downstream for further processing.

In some embodiments, the algae cultivation system 20 includes an ultrasonic apparatus for moving the media 110 relative to the housings 76 in order to cause wiping of the media 110 against the interior surfaces 196 of the housings 76, thereby clearing any accumulated algae from the interior surfaces 196 of the housings 76. The ultrasonic apparatus is controlled by the controller 40 and is capable of operating at a plurality of frequency levels. For example, the ultrasonic apparatus may operate at a relatively low frequency and at a relatively high frequency. Operation of the ultrasonic apparatus at the low frequency may cause movement of the media 110 for purposes of wiping the interior surfaces 196 of the housings 76, but be sufficiently low not to dislodge algae from the media 110. Operation of the ultrasonic apparatus at the high frequency may cause significant or more turbulent movement of the media 110 for purposes of dislodging algae from the media 110 prior to removal of the water and algae from the containers 32. However, operating the ultrasonic apparatus at the high frequency does not damage the algae. For example, the ultrasonic apparatus may operate at the low frequency between a range of about 40 KHz to about 72 KHz and may operate at the high frequency between a range of about 104 KHz to about 400 KHz. These frequency ranges are exemplary ranges only and are not intended to be limiting. Thus, the ultrasonic apparatus is capable of operating at various other frequencies. The algae cultivation system 20 may include a single ultrasonic apparatus for moving the media 110 in all of the containers 32, the system 20 may include a separate ultrasonic apparatus for each of the containers 32, or the system 20 may include any number of ultrasonic apparatuses for moving media 110 in any number of containers 32.

In other embodiments, the algae cultivation system 20 includes other types of devices that are capable of moving the media 110 and/or the frames 108 in order to cause wiping of the media 110 against the interior surfaces 196 of the containers 32 and dislodge the algae from the media 110 in preparation of removal of the water and algae from the containers 32. For example, the algae cultivation system 20 may include a linear translator that moves the frames 108 and media 110 in an up and down linear manner. In such an example, the linear translator is operated in at least two speeds including a slow speed, in which the frames 108 and media 110 are translated at a sufficient rate to cause the media 110 to wipe against the interior surfaces 196 and not cause the algae to be dislodged from the media 110, and a fast speed, in which the frames 108 and media 110 are translated at a sufficient rate to dislodge the algae from the media 110 without damaging the media 110. As another example, the algae cultivation system 20 may include a vibrating device that vibrates the frames 108 and media 110, and is operated in at least two speeds including a slow speed, in which the frames 108 and media 110 are sufficiently vibrated to wipe against the interior surfaces 196 and algae is not dislodged from the media 110, and a fast speed, in which the frames 108 and media 110 are sufficiently vibrated to dislodge the algae from the media 110. The algae cultivation system 20 may include a single vibrating device for moving the media 110 in all of the containers 32, the system 20 may include a separate vibrating device for each of the containers 32, or the system 20 may include any number of vibrating devices for moving media 110 in any number of containers 32.

In yet other embodiments, the algae cultivation system 20 is capable of moving the media 110 and/or the frames 108 in order to cause wiping of the media 110 against the interior surfaces 196 of the containers 32 and dislodge the algae from the media 110 in preparation of removal of the water and algae from the containers 32 by utilizing the gas management system 24. In such embodiments, the gas management system 24 is controllable by the controller 40 to release carbon dioxide and accompanying gases into the containers 32 in at least three manners. The first manner includes a relatively low release of gas in both amount and rate into the containers 32. Gas is released in this first manner during periods of time when normal cultivation of algae is desired. The second manner includes a moderate release of gas into the containers 32. Gas is released in this second manner when sufficient movement of the media 110 is desired to cause the media 110 to wipe against the interior surfaces 196 of the housings 76, but not cause the algae to dislodge from the media 110. The third manner includes a high or turbulent release of gas into the containers 32. Gas is released in this third manner when sufficient movement of the media 110 is desired to dislodge the algae from the media 110.

Referring back to FIG. 49, operation of the flushing system 38 will be described. As indicated above, the flushing system 38 assists with removal of the algae from the media 110. The flushing system 38 may be activated either when the container 32 is full of water or after the water has been exhausted from the container 32. When desired, the controller 40 activates the spray nozzles 43 to spray pressurized water from the nozzles 43 and into the container 32. The spray nozzles 43 may be operable to spray water at a pressure of about 20 psi. Alternatively, the spray nozzles 43 may spray water at a pressure between about 5 psi and about 35 psi. The pressurized water sprays onto the media 110 and removes the algae from the media 110. In some embodiments, the frame 108 and media 110 may be rotated while the spray nozzles 43 are spraying the pressurized water. Rotation of the frame 108 and media 110 moves all of the media 110 within the container 32 in front of the spray nozzles 43 to provide an opportunity for removing the algae from all the media 110 and not just the media 110 immediately in front of the spray nozzles 43.

The flushing system 38 may be utilized in other manners such as, for example, to clean the interior of the container 32 in the event an invasive species or other contaminant has infiltrated the container 32. For example, the container 32 may be drained of any water and algae present therein, the flushing system 38 may be activated to spray water into the container 32 until the container 32 is filled with water, the pH of the water is raised to about 12 or 13 on the pH scale by using sodium hydroxite or other substance to ultimately kill any invasive species or other contaminant in the container 32, the frame 108 and media 110 is rotated in one or both directions to create turbulence in the container 32 and wipe against the inside of the container 32, and then the container 32 is drained. These steps may be repeated until all invasive species or contaminants are eradicated. Next, the flushing system 38 rinses the container 32 by introducing clean water into the container 32 until it is adequately filled, the frame 108 and media 110 are again rotated to create turbulence and wipe against the interior of the container 32, the pH of the water is checked, and the water is drained. The container 32 is ready to be reused for algae cultivation when the water reaches a pH of about 7. The container 32 may require rinsing several times to achieve a pH of 7. In this exemplary operation of the flushing system 38, the container 32 is cleaned without requiring disassembling of the container 32 or other components of the system 20, thereby saving time in the event the container 32 is contaminated.

In other exemplary embodiments, the flushing system 38 may not include the plurality of spray nozzles and instead may include one or more water inlets to introduce water into the container 32 for cleaning and rinsing purposes.

In yet other exemplary embodiments, the water inlet pipe 56 and water inlet 96 already present in the container 32 may be used for introducing water into the container 32 for cleaning and rinsing purposes.

No matter the manner used to dislodge the algae from the media 110, the algae cultivation system 20 is ready to remove the combination of water and algae from the containers 32 after dislodging the algae. To do so, the controller 40 activates the liquid management system 28 to pump the combination of water and algae from the containers 32 via the water outlets 100. Alternatively, water may be drained through opening 88 in the bottom of the container 32. From either or both the opening 88 and/or the water outlets 100, the water and algae are transported downstream via pipes to be processed into fuel such as biodiesel. The initial step of processing may include filtering the algae from the water with a filter. Additional steps may include clarifying and settling the algae after the algae has been extracted from the containers 32. After removal of the water and algae combination from the containers 32, the algae cultivation system 20 can initiate another algae cultivation process by introducing water back into the containers 32 for further cultivation.

The above described algae cultivation process can be considered a cycled cultivation process. Cycled can be characterized by completely filling the containers 32 with water, running a complete cultivation cycle within the containers 32, and completely or substantially draining the water from the containers 32. In some embodiments, the algae cultivation system 20 can perform other types of processes such as, for example, a continuous algae cultivation process. The continuous process is similar in many ways to the cycled algae cultivation process, but has some differences that will be described herein. In a continuous process, the containers 32 are not completely drained to remove the water and algae combination. Instead, a portion of the water and algae are continuously, substantially continuously, or periodically siphoned from the containers 32. In some embodiments, the controller 40 controls the liquid management system 28 to add a sufficient amount of water into the containers 32 through inlets 56 to cause the water level within the containers 32 to rise above the outlets 60 in the containers 32. Water and the algae contained within the water are naturally expelled through the outlets 60 and travel downstream for processing. Introducing sufficient water to cause this overflow of water and algae through the outlets 60 can occur at desired increments or can occur continuously (i.e., the water level is always sufficiently high to cause overflow through outlets 60 in the containers 32). In other embodiments, the controller 40 controls the liquid management system 28 to remove a portion of the water and algae combination from the containers 32 and introduce a quantity of water into the containers 32 substantially equal to the amount removed in order to replace the removed water. This removal and replenishment of water can occur at particular desired increments or can occur continuously. Other manners of controlling the system may be implemented to continuously process algae. Operation of the algae cultivation system 20 in any of these continuous manners decreases algae production down time experienced when all the water and algae are removed from the containers 32 as occurs in the cycled process. In the continuous processes, water is always present in the containers 32 and algae is continuously growing in the water. In some embodiments, the frames 108 and media 110 are rotated at a relatively high speed at desired increments to introduce the algae into the water so that the algae can be expelled from the containers 32 either in an overflow manner described above or in an incremental removal of water manner also described above.

No matter the manner or process used to cultivate algae within the containers 32, the water within the containers 32 may be filtered during the cultivation process to remove metabolic waste produced by the algae during cultivation. High levels of metabolic waste in the water are detrimental to algae cultivation. Accordingly, removal of the metabolic waste from the water improves algae cultivation.

Metabolic waste may be removed from the water in a variety of manners. One exemplary manner includes removing water from the containers 32, filtering the metabolic waste from the water, and returning the water to the containers 32. The system 20 of the present invention facilitates water filtration for purposes of removing the metabolic waste. As indicated above, a large quantity of the algae present in the containers 32 is resting on or adhered to the media 110 present in the containers 32, thereby resulting in a small quantity of algae floating in the water within the containers 32. With small quantities of algae floating in the water, the water can easily be removed from the containers 32 without having to filter large quantities of algae from the water and the potential for loosing, wasting, or prematurely harvesting algae during the filtration process is minimal. Also, with a large quantity of the algae resting on or adhered to the media 110, the algae remains in the container 32 to continue cultivating while the water is being removed, filtered, and reintroduced. It should be understood that this exemplary manner of water filtration is only one of many manners possible for filtering metabolic waste from water and is not intended to be limiting. Accordingly, other manners of water filtration are within the intended spirit and scope of the present invention.

With reference to FIG. 67, operation of the controller 40 with the gas management system 24, liquid management system 28, the container 32, the artificial light system 37, and the ECD 428 will be described. The system 20 includes a light sensor 314, such as, for example, digital light sensor model number TSL2550 manufactured by Texas Instruments, Inc., capable of sensing the amount of light contacting the container 32 and/or amount of light in the environment surrounding the container 32. That is, the sensor 314 can identify whether the container 32 is receiving a significant amount of light (e.g., a sunny day in the summer), a small amount of light (e.g., early in the day, late in the day, cloudy, etc.), or no light (e.g., after sunset or nighttime). The sensor 314 sends a first signal to the motor control 302, which controls the motor 224 of the container 32 to rotate the frame 108 and media 110 dependent on the amount of light received by the container 32. For example, if the container 32 is receiving a significant amount of light, it is desirable to rotate the frame 108 and media 110 at a relatively high rate (but not at a rate that removes the algae from the media 110), and if the container 32 is receiving a low amount of light, it is desirable to rotate the frame 108 and media 110 at a relatively slow rate in order to provide the algae in the container 32 more time to absorb the light. In addition, the sensor 314 sends a second signal to the artificial light control 300, which communicates and cooperates with the ECD control 313 to control the artificial light system 37 and the ECD 428 as necessary to provide a desired amount of light 37, 72 to the container 32. For example, the artificial light system 37 and the ECD 428 may cooperate to activate the light source 41 of the artificial light system 37 and/or the light source 41 of the ECD 428, thereby emitting a desired amount of light onto the container 32 and algae. In low light or no light conditions, it may be desirable to activate the artificial light system 37 and/or the ECD light source 41 to emit light onto the container 32 and algae therein in order to promote the light phase of photosynthesis in times when the light phase may not be naturally occurring due to the lack of natural sunlight 72. Also, for example, in instances where the ambient temperature may be elevated and direct sunlight 72 is not desired due to the resulting rise in temperature, the first and second members 436, 440 of the ECD 428 may be fully closed and one or more of the light sources 41 may be activated to provide a desired quantity of light. Further, for example, the ECD control 313 may control the positions of the first and second members 436, 440 by communicating with the ECD motor 432 to selectively control the exposure of the container 32 to exterior elements (i.e., sunlight and ambient temperature).

With continued reference to FIG. 67, the operational timer 304 of the motor control 302 determines when and how long the motor 224 is activated and deactivated during the algae cultivation process occurring in the container 32. For example, the operational timer 304 determines the rate at which the frame 108 and media 110 will rotate in order to cultivate algae in the container 32. The removal timer 306 determines when and how long the motor 224 will rotate the frame 108 and media 110 to remove algae from the media 110. The removal timer 306 also determines the rate of rotation of the frame 108 and media 110 during the algae removal process. A temperature sensor 316 is disposed within the container 32 to determine the temperature of the water within the container 32 and an ambient temperature sensor 480 is disposed externally of the container 32 to determine the temperature outside of the container 32. As indicated above, proper water temperature is an important factor for effective algae cultivation. The water temperature identified by the temperature sensor 316 and the ambient temperature identified by the ambient temperature sensor 480 are sent to the temperature control 308, which communicates and cooperates with the ECD control 313 to control the temperature control system 45 and/or the ECD 428 as necessary to properly control the water temperature within the container 32. The liquid control 310 controls the liquid management system 28, which controls introduction and exhaustion of liquid into and from the container 32. The gas control 312 controls the gas management system 24, which controls introduction and exhaustion of gas into and from the container 32.

The pH of the water is also an important factor for effectively cultivating algae. Different types of algae demand different pH's for effective cultivation. The system 20 includes a pH sensor 484 that identifies the pH of the water within the container 32 and communicates the identified pH to the liquid control 310. If the pH is at a proper level for algae cultivation within the container 32, the liquid control 310 takes no action. If, on the other hand, the pH of the water is at an undesired level, the liquid control 310 communicates with the liquid management system 28 to take the necessary actions to adjust the pH of the water to the appropriate level. In some exemplary embodiments, the pH sensor 484 may be disposed in external piping through which water is diverted from the container 32 (see FIG. 52). In other exemplary embodiments, the pH sensor 484 may be disposed in the container 32. The pH sensor 484 may be a wide variety of types of sensors. In some exemplary embodiments, the pH sensor 484 may be an ion selective electrode and electrically coupled with the liquid control 310, and the system 20 may include an acid pump, a caustic pump, an acid tank containing acid, and a caustic tank containing caustic. In such embodiments, the caustic pump is activated to pump caustic into the container when the pH level drops below a desired level to raise the pH level to the desired level, and the acid pump is activated to pump acid into the container when the pH level rises above a desired level to lower the pH level to the desired level.

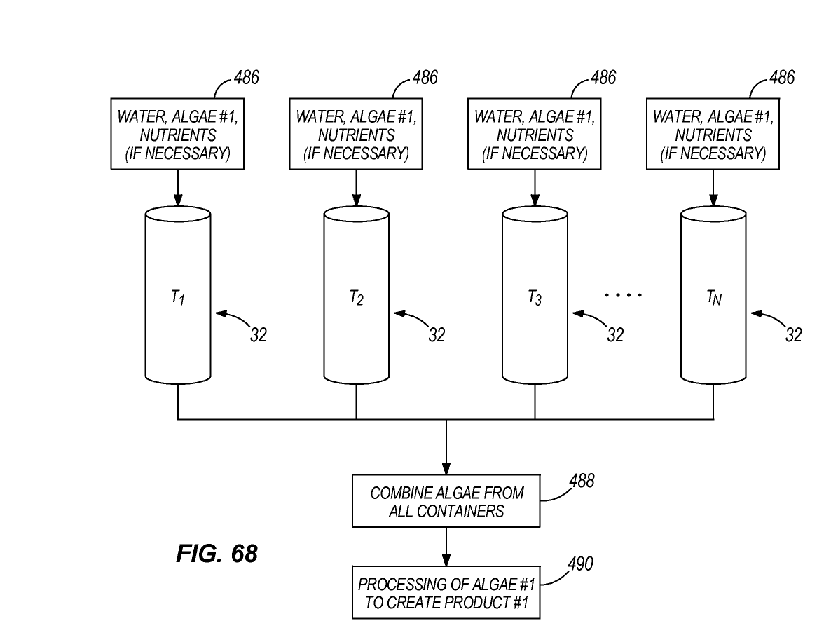

The system 20 may be used in a variety of different manners to achieve a variety of different desired results. The following description relating to FIGS. 68-71 exemplifies a few of the many different uses and operations of the system 20 to achieve a few of the many different desired results. The following exemplary uses and operations are for illustrative purposes and are not intended to be limiting. Many other types of uses and operations are contemplated and are within the spirit and scope of the present invention.

Referring to FIG. 68, a first exemplary operation of the system 20 is illustrated. In this exemplary operation, the system 20 includes a plurality of containers 32. Water, an identical type of algae (represented as algae #1 in the figure), and any necessary nutrients (e.g., carbon dioxide, nitrogen, phosphorus, vitamins, micronutrients, minerals, silica for marine types, etc.) are introduced into each of the containers 32 at step 486. The containers 32 operate in the desired manner(s) to cultivate the algae therein. After completion of the cultivation process, the algae is exhausted from all of the containers 32 and combined together at step 488. The combined quantity of like algae is then forwarded for further processing to create a single type of product (e.g., oil, fuel, comestible items, etc.) at step 490.

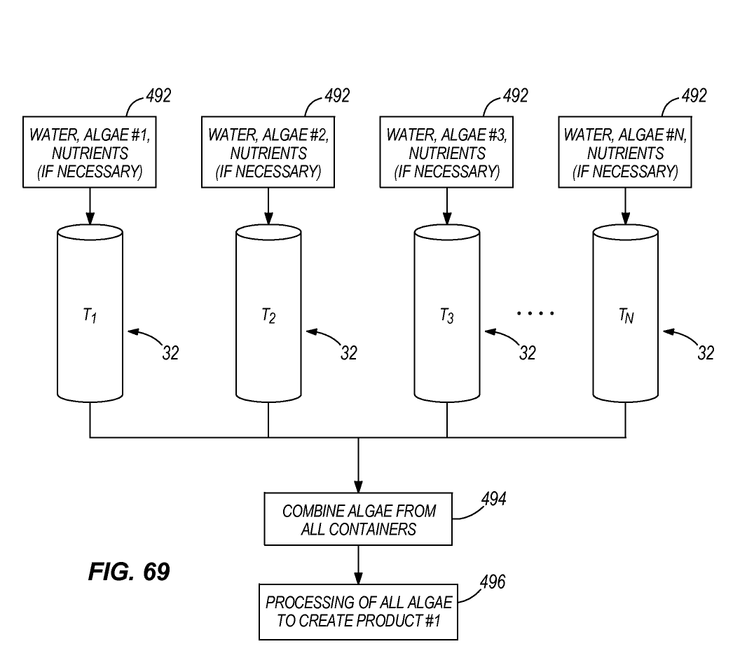

Referring to FIG. 69, a second exemplary operation of the system 20 is illustrated. In this second exemplary operation, the system 20 includes a plurality of containers 32, with each container 32 including water, a different type of algae (represented as algae #1, #2, #3, #N in the figure), and any necessary nutrients for the different types of algae (see step 492). Since this exemplary operation of the system 20 includes different types of algae, different types of nutrients may be introduced into each of the containers 32 as necessary. The containers 32 operate in the desired manners to cultivate the algae therein. Due to the containers 32 having different types of algae therein, the cultivation process of each container 32 may be different in order to efficiently cultivate the specific type of algae. After completion of the cultivation processes of the containers 32, the algae is exhausted from all of the containers 32 and combined together at step 494. The combined quantity of different types of algae is then forwarded for further processing to create a single type of product 496.

Referring to FIG. 70, a third exemplary operation of the system 20 is illustrated. In this third exemplary operation, the system 20 includes a plurality of containers 32, with each container 32 including water, an identical type of algae (represented as algae #1 in the figure), and any necessary nutrients necessary for algae cultivation (see step 498). The containers 32 operate in the desired manner(s) to cultivate the algae therein. After completion of the cultivation process, the algae from each container 32 is exhausted and remains segregated from algae exhausted from the other containers 32 at step 500. Even though the quantity of exhausted algae from each container 32 is the same type of algae, the quantities of algae from the containers 32 are independently forwarded for further processing to create independent products (products #1, #2, #3, and #N in the figure) at step 502.

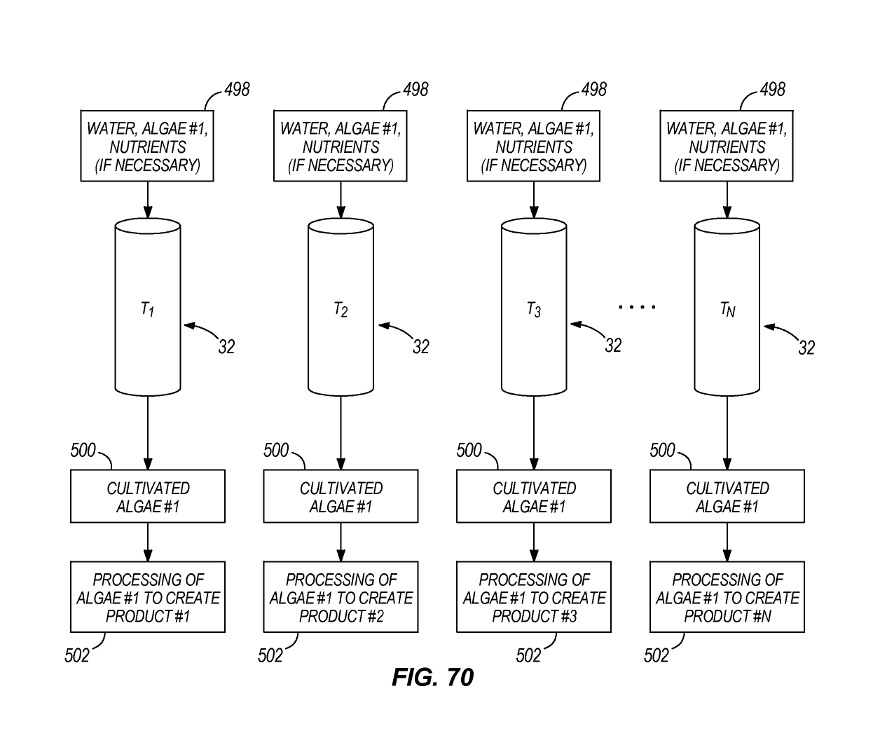

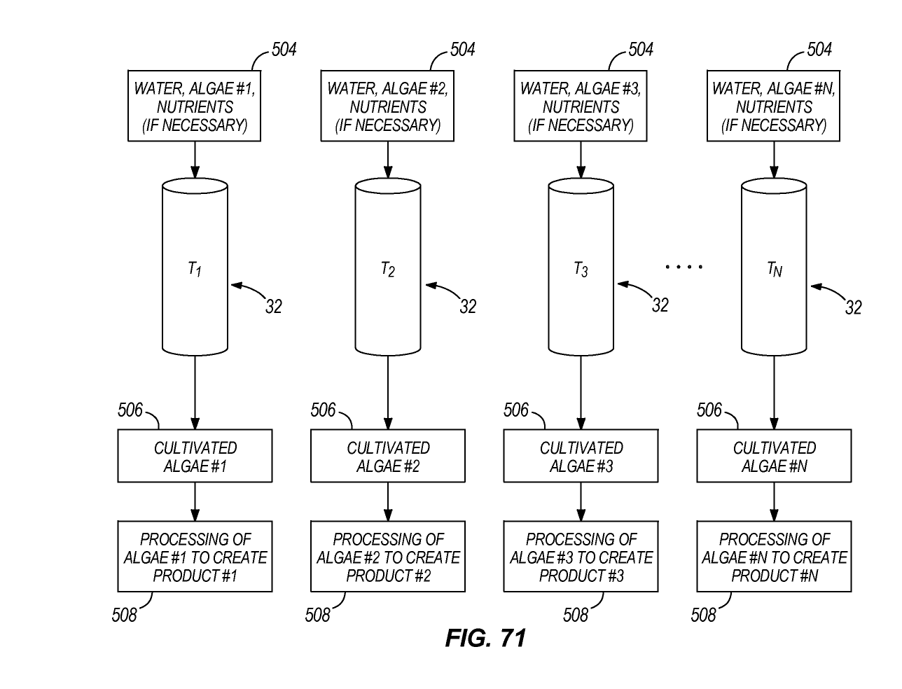

Referring to FIG. 71, a fourth exemplary operation of the system 20 is illustrated. In this fourth exemplary operation, the system 20 includes a plurality of containers 32, with each container 32 including water, a different type of algae (represented as algae #1, #2, #3, #N in the figure), and any necessary nutrients for the different types of algae (see step 504). Since this exemplary operation of the system 20 includes different types of algae, different types of nutrients may be introduced into each of the containers 32 as necessary. The containers 32 operate in the desired manners to cultivate the algae therein. Due to the containers 32 having different types of algae therein, the cultivation process of each container 32 may be different in order to efficiently cultivate the specific type of algae. After completion of the cultivation processes of the containers 32, the algae from each container 32 is exhausted and remains segregated from algae exhausted from the other containers 32 at step 506. The quantities of different algae from the containers 32 are independently forwarded for further processing to create independent products (products #1, #2, #3, and #N in the figure) at step 508.

Referring now to FIGS. 72-75, the containers 32 are capable of having a variety of different shapes such as, for example, square, rectangular, triangular, oval, or any other polygonal or arcuately-perimetered shape and having complimentarily shaped components to cooperate with the shape of the containers 32. Containers 32 having these or other shapes are capable of performing in the same manners as the round containers 32 described herein. In addition, the frames 108 and media 110 are movable to wipe the interior surfaces 196 of the housings 76. For example, the frames 108 and media 110 may be moved back-and-forth along a linear path to wipe the interior surfaces 196. Such linear movement may be parallel to the longitudinal axis of the containers 32 (i.e., up and down), perpendicular to the longitudinal axis (i.e., right to left), or some other angle relative to the longitudinal axis of the containers 32. Movement of the frames 108 and media 110 in these manners may be performed by a DC cycling motor capable of switching polarity during the cycle in order to provide the back-and-forth movement. Alternatively, a motor may be connected to a mechanical linkage that facilitates the back-and-forth movement.

The following are exemplary production scenarios to illustrate exemplary capabilities of the algae cultivation system 20. This example is provided for illustrative purposes and is in no way intended to be limiting upon the capabilities of the system 20 or upon the manner the system 20 is used to cultivate algae. Other exemplary production scenarios are contemplated and are within the intended scope of the present invention.

A container 6-feet tall by 3-inches in diameter contains approximately 100 feet of media and is filled with approximately 8.32 liters (2.19 gallons) of water seeded with Chlorella Vulgaris algae. The container and associated components operate for approximately 7 days. The frame and media are rapidly rotated to dislodge the C. Vulgaris algae from the media and the algae is drained from the container. Approximately 400 ml of concentrated algae settled out in 2 days from the 8.32 liters (2.19 gallons) of cultivated water. The container is refilled with 8.32 liters (2.19 gallons) of fresh water and the algae remaining in the container (seeding algae) is allowed to cultivate for 6 days. After 6 days, the frame and media are rapidly rotated to dislodge the algae, and the algae and water are exhausted from the container. This time, the 8.32 liters (2.19 gallons) of cultivated water produce 550 ml of concentrated algae. From these data, it may be estimated that one-hundred 8.32 liter (2.19 gallon) containers may produce 55 liters (14.5 gallons) of concentrated algae every 6 days.

Another exemplary production scenario includes thirty (30) containers, each of which is 30-feet tall by 6-feet in diameter, has a footprint of 28.3 ft², and a volume of 850 ft³. Thus, all thirty containers provide a total volume of about 25,500 ft³ and cover an area of about 17,000 ft² (or about 0.40 acres). Carbon dioxide is introduced into the containers in a feed stream comprising approximately 12% of carbon dioxide by volume. The algae yield for this exemplary scenario is 4 grams of algae per liter per day, which results in an annual production (assuming 90% utilization of the thirty containers) of approximately 1000 tons of algae and consumption of approximately 2000 tons of carbon dioxide per year.

The foregoing description has been presented for purposes of illustration and description, and is not intended to be exhaustive or to limit the invention to the precise form disclosed. The descriptions were selected to explain the principles of the invention and their practical application to enable others skilled in the art to utilize the invention in various embodiments and various modifications as are suited to the particular use contemplated. Although particular constructions of the present invention have been shown and described, other alternative constructions will be apparent to those skilled in the art and are within the intended scope of the present invention.

What is claimed is:

1. A container for cultivating a microorganism, comprising:
a housing for containing water and the microorganism;
an inlet defined in the housing for permitting gas to enter the housing; and
a media at least partially positioned within the housing and including an elongated member and a plurality of loop members extending from the elongated member.

2. The container of claim 1, wherein the inlet permits carbon dioxide to enter the housing.

3. The container of claim 1, wherein the elongated member is a central core of the media and the plurality of loop members extend from two opposite sides of the central core.

4. The container of claim 1, wherein the media is one of a plurality of medias, and wherein the plurality of medias extend in a substantially vertical direction and are spaced apart from one another.

5. A container for cultivating a microorganism, comprising:
a housing for containing water and the microorganism;
an inlet defined in the housing for permitting gas to enter the housing;
a frame at least partially positioned within the housing and including a first portion and a second portion, wherein the first portion is spaced apart from the second portion; and
a media at least partially positioned within the housing and supported by and extending between the first and second portions.

6. The container of claim 5, wherein the first portion is a first substantially cylindrical plate and the second portion is a second substantially cylindrical plate, the frame further including a shaft extending between and coupled to the first and second spaced apart plates.

7. The container of claim 5, wherein the media is one of a plurality of medias spaced apart from one another, and wherein the plurality of medias are supported by and extend between the first and second portions of the frame.

8. A container for cultivating a microorganism, comprising:
a housing for containing water and a microorganism; and
a media positioned within the housing and in contact with an interior surface of the housing, wherein the media is movable between a first position and a second position within the housing, and wherein the media maintains contact with the interior surface of the housing as the media moves between the first and second positions.

9. The container of claim 8, wherein the media is rotatable between the first position and the second position.

10. The container of claim 8, further comprising a frame and a drive member coupled to the frame, wherein the media is supported by the frame, and wherein the drive member is adapted to move the frame and the media between the first position and the second position.

11. The container of claim 10, wherein the frame includes a first portion and a second portion spaced apart from one another, the first portion including a first periphery and the second portion including a second periphery, wherein the media is supported by and extends between the first and second portions near the first and second peripheries of the first and second portions.

12. The container of claim 11, wherein the first and second peripheries of the first and second portions of the frame are positioned near the interior surface of the housing to contact the interior surface of the housing with the media.

13. A method for cultivating a microorganism, comprising the steps of:
providing a container for containing water and the microorganism;
positioning a media at least partially within the container and in contact with an interior surface of the container;
moving the media within the container from a first position to a second position; and
maintaining the media in contact with the interior surface of the housing as the media moves from the first position to the second position.

14. The container of claim 13, wherein moving the media within the container comprises rotating the media within the container from the first position to the second position.

15. A container for cultivating a microorganism, comprising:
a housing for containing water and the microorganism;
a frame at least partially positioned within the housing and including a first portion and a second portion, wherein the first portion is spaced apart from the second portion, and wherein the frame is rotatable relative to the housing;
a first media segment coupled to and extending between the first and second portions of the frame; and
a second media segment coupled to and extending between the first and second portions of the frame, wherein at least a portion of the first media segment and at least a portion of the second media segment are spaced apart from each other.

16. The container of claim 15, wherein the first media segment and the second media segment are comprised of a single unitary media.

17. The container of claim 15, wherein the first media segment and the second media segment are two distinct, separate medias.

18. The container of claim 15, wherein the first and second media segments extend between the first and second portions in a first direction, and wherein the at least a portion of the first media segment and the at least a portion of the second media segment are spaced apart from each other in a second direction transverse to the first direction.

19. The container of claim 18, wherein the first direction is a vertical direction.

20. A container for cultivating a microorganism, comprising:
a housing for containing water and the microorganism, the housing including a sidewall;
a plurality of media segments at least partially positioned within the housing and including a first pair of media segments spaced apart from each other a first distance and a second pair of media segments spaced apart from each other a second distance, wherein the first distance is greater than the second distance, and wherein the first pair of media segments is positioned closer to the sidewall than the second pair of media segments.

21. The container of claim 20, wherein the media segments are comprised of a single unitary media.

22. The container of claim 20, wherein the media segments are comprised of distinct, separate medias.

23. A container for cultivating a microorganism, comprising:
a housing for containing water and the microorganism;
a frame at least partially positioned within the housing and including two spaced apart frame portions; and
a media at least partially positioned within the housing and extending between the two spaced apart frame portions, wherein the frame is constructed of a first material more rigid than a second material of which the media is constructed.

24. A container for cultivating a microorganism, comprising:
a housing for containing water and the microorganism;
a frame at least partially positioned within the housing and movable relative to the housing;
a drive member coupled to the frame and adapted to move the frame at a first speed and a second speed, wherein the first speed is different than the second speed; and
a media at least partially positioned within the housing and coupled to the frame.

25. The container of claim 24, wherein the frame is rotatable relative to the housing.

26. The container of claim 24, wherein the frame is translatable relative to the housing.

27. A container for cultivating a microorganism, comprising:
a housing for containing water and the microorganism;
a frame at least partially positioned within the housing and movable relative to the housing, the frame including two spaced apart frame portions;
a drive member coupled to the frame for moving the frame; and
a media at least partially positioned within the housing and extending between the two spaced apart frame portions.

28. The container of claim 27, wherein the frame is rotatable relative to the housing.

29. The container of claim 27, wherein the frame is translatable relative to the housing.

30. A container for cultivating a microorganism, comprising:
a housing for containing water and the microorganism;
a frame at least partially positioned within the housing and movable relative to the housing;
a media coupled to the frame; and
an artificial light source for emitting light into an interior of the housing.

31. The container of claim 30, wherein the artificial light source is positioned outside of the housing.

32. The container of claim 30, wherein the artificial light source is positioned in the interior of the housing.

33. The container of claim 30, wherein the artificial light source is a first artificial light source, the container further comprising a second artificial light source for emitting light into the interior of the housing, and wherein the first artificial light source is positioned outside the housing and the second artificial light source is positioned in the interior of the container.

34. A container for cultivating a microorganism, comprising:
a housing for containing water and the microorganism;
an artificial light source for emitting light into an interior of the housing;
a member associated with the artificial light source and through which the light emitted from the artificial light source passes; and
a wiping element at least partially positioned within the housing and in contact with the member, wherein the wiping element is movable relative to the member to wipe against the member.

35. The container of claim 34, wherein the member is a sidewall of the housing.

36. The container of claim 34, wherein the member is a light element positioned in the interior of the housing.

37. The container of claim 36, wherein the light element is substantially cylindrical, has a height dimension greater than a diameter dimension, and extends in a substantially vertical direction within the housing.

38. The container of claim 36, wherein the light element is substantially cylindrical in shape and has a height dimension smaller than a diameter dimension and is positioned in a substantially horizontal plane across the housing.

39. The container of claim 34, wherein the member is a hollow transparent tube positioned in the interior of the housing, and wherein the artificial light source is positioned within the hollow transparent tube.

40. The container of claim 34, further comprising a drive member coupled to the wiping element for moving the wiping element.

41. A container for cultivating a microorganism, comprising:
a housing for containing water and the microorganism and including a sidewall, wherein the sidewall permits sunlight to pass therethrough to an interior of the housing;
an artificial light source associated with the housing for emitting light into an interior of the housing;
a sensor associated with the housing for sensing a quantity of sunlight passing through the sidewall and into the interior of the housing; and
a controller electrically coupled to the sensor and the artificial light source, wherein the controller is capable of activating the artificial light source when the sensor senses a less than desired quantity of sunlight passing into the interior of the housing.

42. A container for cultivating a microorganism, comprising:
a housing for containing water and the microorganism; and
a reflective element positioned outside of the housing for directing light toward an interior of the housing.

43. The container of claim 42, wherein the reflective element is arcuately shaped and surrounds at least a portion of the housing.

44. A method for cultivating microorganisms, comprising the steps of:
providing a container which contains water and includes a media at least partially positioned within the container, wherein the media includes an elongated member and a plurality of loop members extending from the elongated member;
cultivating microorganisms within the container;
removing the water and a first portion of the microorganisms from the container and leaving a second portion of the microorganisms on the media;
refilling the container with water which does not contain the microorganisms; and
cultivating microorganisms in the refilled container from the second portion of microorganisms that remained on the media.

45. The method of claim 44, wherein the elongated member is a central core of the media and the plurality of loop members extend from two opposite sides of the central core.

46. The method of claim 44, wherein providing a container further includes providing a container which includes a plurality of medias at least partially positioned within the container.

47. A method for cultivating microorganisms, comprising the steps of:
providing a container which contains water and includes a media at least partially positioned within the container;
cultivating microorganisms within the container;
removing substantially all of the water and a first portion of the microorganisms from the container and leaving a second portion of the microorganisms on the media;
refilling the container with water which does not contain the microorganisms; and
cultivating microorganisms in the refilled container from the second portion of microorganisms that remained on the media.

48. The method of claim 47, wherein providing a container further includes providing a container which includes a plurality of medias at least partially positioned within the container.

49. A method for cultivating microorganisms, comprising the steps of:
providing a housing having a height dimension greater than a width dimension;
positioning water into the container through a water inlet associated with the container;
positioning a gas into the container through a gas inlet associated with the container;
providing a plurality of media segments in the container, wherein the plurality of media segments extend in a generally vertical direction and are spaced apart from one another; and
cultivating microorganisms in the container, wherein a first concentration of the microorganisms is supported by the plurality of media segments and a second concentration of microorganisms is suspended in the water, wherein the first concentration of microorganisms is greater than the second concentration of microorganisms.

50. The method of claim 49, wherein the media segments are comprised of a single unitary media.

51. The method of claim 49, wherein the media segments are comprised of distinct, separate medias.

52. A container for cultivating microorganisms, comprising:
a housing having a height dimension greater than a width dimension, the housing adapted to contain water and the microorganisms;
a gas inlet associated with the housing for introducing gas into the container;
a water inlet associated with the housing for introducing water into the container; and
a plurality of media segments at least partially positioned within the housing, extending in a generally vertical direction, and spaced apart from one another, wherein a first concentration of the microorganisms is supported by the plurality of media segments and a second concentration of microorganisms is suspended in the water, wherein the first concentration of microorganisms is greater than the second concentration of microorganisms.

53. The container of claim 52, wherein the media segments are comprised of a single unitary media.

54. The container of claim 52, wherein the media segments are comprised of distinct, separate medias.

55. The container of claim 52, wherein the housing is at least partially transparent to permit light to pass therethrough toward an interior of the housing.

56. A system for cultivating microorganisms, comprising:
a first container for containing water and cultivating microorganisms within the first container;
a second container for containing water and cultivating microorganisms within the second container; and
a conduit interconnecting the first container and the second container for carrying a gas out of the first container and into the second container.

57. The system of claim 56, wherein the conduit is a first conduit, the system further comprising a gas source and a second conduit, the second conduit coupling the gas source to the first container to permit gas to travel from the gas source to the first container.

58. A container for cultivating a microorganism, comprising:
a housing for containing water and the microorganism;
a first opening defined in the housing through which water is introduced into the housing at a first pressure; and
a second opening defined in the housing through which water is introduced into the housing at a second pressure, wherein the first pressure is greater than the second pressure.

59. The container of claim 58, wherein the first opening is utilized to introduce water into an interior of the housing for cleaning the housing and the second opening is utilized to introduce water into the housing for cultivating the microorganism.

60. A method for cultivating microorganisms, comprising:
providing a housing including a first opening and a second opening;
cultivating microorganisms in the housing;
introducing water into the housing through the first opening at a first pressure; and
introducing water into the housing through the second opening at a second pressure, wherein the first pressure is greater than the second pressure.

61. The method of claim 60, wherein introducing water into the housing through the first opening further comprises cleaning an interior of the housing by introducing water into the housing through the first opening at a first pressure, and wherein introducing water into the housing through the second opening occurs prior to cultivating microorganisms into the housing.

62. A system for cultivating microorganisms, comprising:
a container for containing water and the microorganisms; and
a conduit for containing a fluid, wherein the conduit is positioned to contact the water of the container, and wherein a temperature of the fluid differs from a temperature of the water for changing the temperature of the water.

63. The system of claim 62, wherein the conduit is positioned completely outside the container.

64. The system of claim 62, wherein the conduit is at least partially positioned within the container.

65. The system of claim 62, wherein the conduit is a first conduit, the system further comprising a second conduit for containing a fluid, wherein the second conduit is positioned to contact the water of the container, and wherein a temperature of the fluid differs from a temperature of the water for changing the temperature of the water.

66. The system of claim 65, wherein the first conduit is positioned at least partially within the container near a top of the container and the second conduit is positioned at least partially within the container near a bottom of the container.

67. A method for cultivating microorganisms, comprising the steps of:
providing a container for containing water;
positioning a frame at least partially within the container;
coupling media to the frame;
cultivating microorganisms on the media within the container;
moving the frame and the media at a first speed;
moving the frame and the media at a second speed different than the first speed;
removing a portion of the water containing cultivated microorganisms from the container; and
introducing additional water into the container to replace the removed water.

68. The method of claim 67, further comprising providing a drive member coupled to the frame for moving the frame and media at the first and second speeds.

69. The method of claim 68, wherein moving the frame and media at the first and second speeds further comprises rotating the frame and media at the first and second speeds.

70. A system for cultivating microorganisms, comprising:
a first container for containing water and for cultivating a first species of microorganism therein;
a second container for containing water and for cultivating a second species of microorganism therein, wherein the first species of microorganism is different than the second species of microorganism;
a first conduit connected to the first container for carrying gas to the first container originating from a gas source; and
a second conduit connected to the second container for carrying gas to the second container originating from the gas source.

71. The system of claim 70, wherein the second conduit is also connected to the first container and carries gas from the first container to the second container, and wherein at least a portion of the gas carried from the first container to the second container via the second conduit originated from the gas source.

72. The system of claim 70, wherein the first species of microorganisms cultivated is utilized to manufacture a first product and the second species of microorganisms cultivated is utilized to manufacture a second product different than the first product.

73. The system of claim 70, wherein the first and second species of microorganisms cultivated are combined to manufacture a single type of product.

74. A system for cultivating microorganisms, comprising:
a first container for containing water and for cultivating microorganisms of a first species;
a second container for containing water and for cultivating microorganism of the first species;
a first conduit connected to the first container for carrying gas to the first container originating from a gas source; and
a second conduit connected to the second container for carrying gas to the second container originating from the gas source, wherein a first portion of the microorganisms cultivated is utilized to manufacture a first product and a second portion of the microorganisms cultivated is utilized to manufacture a second product.

75. A system for cultivating microorganisms, comprising:
a first container for containing water and for cultivating a first species of microorganism therein;
a second container for containing water and for cultivating a second species of microorganism therein, wherein the first species of microorganism is different than the second species of microorganism;
a first conduit connected to the first container for carrying gas to the first container, wherein the gas originates from a gas source; and
a second conduit connected to the second container for carrying gas to the second container, wherein the gas originates from the gas source, and wherein the first species of microorganism cultivated in the first container is utilized to manufacture a first product and the second species of microorganism cultivated in the second container is utilized to manufacture a second product.

76. A container for cultivating a microorganism, comprising:
a housing for containing water and the microorganism, the housing including a sidewall for permitting light to pass to an interior of the housing; and
an ultraviolet inhibitor associated with the sidewall for inhibiting at least one wave length of light from passing through the sidewall.

77. The container of claim 76, wherein the ultraviolet inhibitor is formed separately from and coupled to the housing.

78. The container of claim 76, wherein the housing and the ultraviolet inhibitor are unitarily formed during production thereof.

79. A method for harvesting free oxygen during cultivation of microorganisms, the method comprising the steps of:
providing a container for containing water, the container including a frame and a media supported by the frame;
introducing gas into the container;
cultivating microorganisms within the container;
moving the frame and media with a drive member to dislodge free oxygen from the media, wherein the free oxygen is generated from cultivating the microorganisms; and
removing the dislodged free oxygen from the container.

80. The method of claim 79, wherein moving the frame and media further comprises rotating the frame and media.

81. The method of claim 79, wherein cultivating microorganisms further includes rotating the frame and the media with the drive member at a first speed during microorganism cultivation, and wherein moving the frame and media with the drive member to dislodge the free oxygen further includes rotating the frame and the media with the drive member at a second speed to dislodge free oxygen, and wherein the second speed is faster than the first speed.

82. A system for cultivating microorganisms, comprising:
a first container for containing water and microorganisms, wherein the first container includes a vertical dimension greater than a horizontal dimension;
a second container for containing water and microorganisms, wherein the second container includes a vertical dimension greater than a horizontal dimension, and wherein the second container is positioned above the first container;
a gas source providing a gas to the first and second containers for facilitating cultivation of the microorganisms within the first and second containers; and
a water source providing the water to the first and second containers for facilitating cultivation of the microorganisms within the first and second containers.

83. A container for cultivating microorganisms, comprising:
a housing for containing water and microorganisms;
a frame at least partially positioned within the housing and including a first portion spaced apart from a second portion;
a first media segment coupled to and extending between the first and second portions of the frame, wherein a first portion of the microorganisms is supported by the first media segment; and
a second media segment coupled to and extending between the first and second portions of the frame, wherein a second portion of the microorganisms is supported by the second media segment, and wherein the first media segment is spaced apart from the second media segment.

84. The container of claim 83, wherein the first media segment and the second media segment are comprised of a single unitary media.

85. The container of claim 83, wherein the first media segment and the second media segment are two distinct, separate medias.

86. A container for cultivating a microorganism, comprising:
a housing for containing water and the microorganism;
a frame at least partially positioned within the housing;
a drive member coupled to the frame to move the frame;
a media supported by the frame and providing support for the microorganism during cultivation; and
an artificial light source for providing light to an interior of the housing.

87. A container for cultivating a microorganism, comprising:
a housing for containing water and the microorganism;
a frame at least partially positioned within the housing;
a media supported by the frame and providing support for the microorganism during cultivation;
a first artificial light source for providing light to an interior of the housing; and
a second artificial light source for providing light to the interior of the housing, wherein the first and second artificial light sources are separate light sources.

88. The container of claim 87, wherein the first artificial light source is positioned within the interior of the housing and the second artificial light source is positioned externally of the housing.

89. The container of claim 87, wherein the first artificial light source is positioned within and at a center of the interior of the housing and the second artificial light source is positioned within the interior of the housing and spaced apart from the center of the housing.

90. A container for cultivating a microorganism, comprising:
a housing for containing water and the microorganism;
a frame at least partially positioned within the housing;
a media supported by the frame and providing support for the microorganism during cultivation; and
an artificial light source disposed externally of the housing and for providing light to an interior of the housing, wherein the artificial light source includes a member and a lighting element coupled to the member for emitting light, and wherein the member is movable toward and away from the housing.

91. A container for cultivating a microorganism, comprising:
a housing for containing water and the microorganism;
an at least partially opaque outer wall coupled to the housing and at least partially surround the housing, wherein the at least partially opaque outer wall inhibits light from passing therethrough and into an interior of the housing;
a frame at least partially positioned within the housing;
a media supported by the frame and providing support for the microorganism during cultivation; and
a light element coupled to the housing and the outer wall to transmit light from an exterior of the container to an interior of the housing.

92. A container for cultivating a microorganism, comprising:
an at least partially opaque housing for containing water and the microorganism, wherein the at least partially opaque housing inhibits light from passing therethrough and into an interior of the housing;
a frame at least partially positioned within the housing;
a media supported by the frame and providing support for the microorganism during cultivation; and
a light element coupled to the housing to transmit light from an exterior of the housing to an interior of the housing.

93. A container for cultivating a microorganism, comprising:
a housing for containing water and the microorganism; and
a member positioned outside of the housing and movable relative to the housing between a first position, in which the member at least partially surrounds a first portion of the housing, and a second position, in which the member at least partially surrounds a second portion of the housing, wherein the first portion is greater than the second portion.

94. A method for cultivating a microorganism, comprising the steps of:
providing a container for containing water and the microorganism, the container including a media at least partially positioned within the container;
cultivating the microorganism on the media;
removing at least a portion of the water from the container while retaining the microorganism on the media; and
replacing at least a portion of the water removed back into the container.

95. The method of claim 94, further comprising treating the portion of the water removed prior to replacing at least a portion of the water back into the container.

96. A container for cultivating a microorganism, comprising:
a housing for containing water and the microorganism;
an inlet defined in the housing for permitting gas to enter the housing;
a valve associated with the inlet which regulates the flow of gas into the housing;
a pH sensor at least partially positioned within the housing to sense a pH level of water contained in the housing; and
a controller electrically coupled to the valve and the pH sensor, wherein the controller controls the valve dependent on a pH level of the water sensed by the pH sensor.

97. The container of claim 96, wherein the gas comprises carbon dioxide.

98. A container for cultivating a microorganism, comprising:
a housing for containing water and the microorganism; and
a frame at least partially positioned within the housing and including a float device for providing buoyancy to the frame.

99. The container of claim 98, wherein at least a portion of the frame is submerged in water contained within the housing and the float device floats on the water.

100. The container of claim 98, wherein the float device is positioned near a top of the frame.

101. The container of claim 98, wherein the frame includes a first portion and a second portion spaced apart from one another, the container further comprising a media at least partially positioned within the housing, coupled to the frame and extending between the first and second portions of the frame, and wherein the float device is positioned above the media.
